(19) United States
(12) Patent Application Publication
(10) Pub. No.: US 2004/0152108 A1
(43) Pub. Date: Aug. 5, 2004

(54) Method for sequence analysis

(76) Inventors: Jonathan MacGregor Keith, Queensland (AU); Darryn Edward Bryant, Queensland (AU); Peter Adams, Queensland (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/673,938

(22) Filed: Sep. 29, 2003

Related U.S. Application Data
(63) Continuation of application No. PCT/AU02/00397, filed on Mar. 28, 2002.
(60) Provisional application No. 60/279,238, filed on Mar. 28, 2001.

(51) Int. Cl. C12Q 1/68; G06F 19/00; G01N 33/48; G01N 33/50

(52) U.S. Cl. 435/6; 702/20

(57) ABSTRACT

The present invention is directed to the use of at least one secondary subunit sequence that varies from a primary subunit sequence by the addition, deletion and/or substitution of at least one subunit for inferring information about the primary subunit sequence. The invention is also directed to the use of such variant sequences for wholly or partially executing a task on an primary subunit sequence, which in one embodiment is refractory to the execution of that task and more particularly to the use of such sequence(s) for wholly or partially deducing the sequence of a primary subunit sequence. In a preferred embodiment of the present invention, the variant sequences are generated by mutagenesis techniques. The subject invention further relates to a method, which is optionally implemented by a processing system, for designing secondary subunit sequences. The present invention further extends to a method, which is suitably implemented by a processing system, to a computer program product for comparing subsequences derived from a primary subunit sequence and from at least one secondary subunit sequence to facilitate inference of the primary subunit sequence.

### RELATED APPLICATIONS

This application is a continuation of International Application No. PCT/AU02/00397, filed Mar. 28, 2002 and published in English, the U.S. designation of which is a non-provisional application of U.S. Provisional Application No. 60/279,238, filed Mar. 28, 2001.

### FIELD OF THE INVENTION

This invention relates generally to a method of sequence analysis. More particularly, the present invention relates to the construction of at least one secondary subunit sequence that varies from a primary subunit sequence by the addition, deletion and/or substitution of at least one subunit and to its use for inferring information about the primary subunit sequence. In one embodiment, the secondary subunit sequence(s) is used for analyzing the refractory behavior of the primary subunit sequence to the execution of a task thereon. The invention also relates to the use of one or more such secondary subunit sequences for wholly or partially executing a task on a primary subunit sequence, which in one embodiment is refractory to the execution of that task, and for wholly or partially deducing the sequence of a primary subunit sequence. The invention also extends to a whole or partial primary subunit sequence so deduced. The present invention is further directed to one or more subsequences that are derived from the primary subunit sequence, and to one or more subsequences that are derived from the at least one secondary subunit sequence. The instant invention further relates to a method of producing such secondary subunit sequences, and to their use in deriving a set of subsequences for comparison with a set of subsequences derived from the primary subunit sequence to facilitate the deduction of the primary subunit sequence. The subject invention further relates to a method, which is optionally implemented by a processing system, for designing secondary subunit sequences as well as to a method and to a computer program product for analyzing subsequences derived from a primary subunit sequence and from at least one secondary subunit sequence to facilitate deduction of the primary subunit sequence.

Bibliographic details of various publications numerically referred to in this specification are collected at the end of the description.

### BACKGROUND OF THE INVENTION

A large industrial effort has been applied recently in the field of biotechnology to generate the entire genomic sequence of animals and plants. Much of this sequence information has been generated by high throughput automated sequencing of large DNA fragments derived from an organism of interest. Although a first pass approximation of the human genome sequence has been reported (IHGSC, 2001 Nature 409, 860-921), this draft sequence contains many gaps, including deletions, errors and omissions. It is clear, therefore, that this draft sequence does not represent a completed and accurate sequence and, accordingly, substantial re-sequencing is required to elucidate an accurate representation of the human genomic sequence. Thus, additional sequencing of chromosomal sequences is required to close gaps, resolve ambiguities, and to ideally ensure that a genome database contains less than a single error for every 10,000 bases of chromosomal sequence. In order to reach this level of completeness and accuracy, however, it is estimated that the entire length of each chromosome will need to be resequenced between 8 and 9 times. Currently this level of completeness and accuracy is only available for human chromosomes 21 and 22.

Large portions of the human genome have been sequenced by shotgun sequence analysis, which is a high throughput enzymatic protocol based on sequencing randomly selected short fragments of target DNA and assembling them to reconstruct the target. The sum of the fragment lengths is generally several times the target length. This multiple coverage facilitates reconstruction and reduces the number of gaps in the reconstructed sequence. Shotgun sequencing is the most common method for sequencing long DNA clones, from around 30 kb (cosmid clones) up to around 150 kb (BAC clones). It has also been used to sequence entire genomes, and is the basis of a commercial approach to sequencing the human genome (Venter et al, 2001). Shotgun reconstruction is complicated by the presence of repeated motifs in the target, which lead to assembly ambiguities. Analyzes of the whole-genome shotgun approach, with particular reference to the problems posed by repeats, are provided by Weber and Myers (1997) and Siegel et al. (2000).

At the present time there are very few alternatives to high throughput enzymatic protocols for sequencing nucleic acids and, accordingly, there is a need for more rapid and effective means for sequencing large regions of DNA rapidly, accurately and cost effectively. Sequencing by Hybridization (SBH) is an example of an alternative sequencing technology that has the potential to meet the needs of accurate and useful industrial sequencing. SBH utilizes hybridization as a means of providing sequence information. In general, the SBH method is based upon the ability of a single stranded nucleic acid molecule to form an anti-parallel complex with a complementary single stranded nucleic acid probe. SBH involves hybridization of a target nucleic acid molecule to a set of oligonucleotide probes of a length shorter that the target molecule wherein each probe has the potential to represent a contiguous complementary sequence of the target molecule. SBH of a target nucleic acid molecule can be visualized as consisting of two steps; 1) a process of dissolving the target nucleic acid molecule into all its constituent oligonucleotide p-mers, and 2) the back assembly of p-mers detected by hybridization and assembled by overlap into an extended sequence. Hybridization of all possible p-mer oligonucleotide probes to the target nucleic acid molecule determines the p-mer oligonucleotide subset contained in the primary sequence of the target nucleic acid. Positively hybridizing p-mer oligonucleotide probes are ordered and the sequence of the target DNA is determined using (p−1)-mer overlapping frames between the oligonucleotide probes.

The provision of DNA microfabricated arrays (micro-arrays) has facilitated an ‘order of magnitude’ increase in speed and specificity for SBH analysis. For example, reference may be made to Southern (WO89/10977; U.S. Pat. No. 6,045,270), Chee et al. (U.S. Pat. No. 5,837,832) Cantor et al. (U.S. Pat. No. 6,007,987), and Fodor et al. (U.S. Pat. No. 5,871,928). These arrays typically consist of a fixed pattern (e.g., a matrix) of positionally defined regions with attached sequence-specific probes (e.g., oligonucleotide probes) for specifically binding to a predetermined subunit sequence of a preselected multi-subunit length having at least three subunits. For subunit sequences based on a four-letter alphabet such as nucleic acid sequences, a complete set of 4^{p} probes of length p is commonly synthesized and arranged in a fixed pattern on an array. Typically, fluorescently tag copies of an unknown target nucleic acid molecule are then hybridized to the probe array. This permits the precise identification of oligonucleotide sequences that are complementary to the unknown sequence. A major advantage of SBH is that it is highly parallel in nature, simultaneously detecting all probes that hybridize to the target fragment (Gunderson et al., 1998; Pevzner and Lipshutz, 1994; Ramsay, 1998; Lipshutz et al., 1999; Pe'er and Shamir, 2000; Drmanac, 2000). In contrast, conventional protocols of sequencing define a nucleic acid sequence in a base-by-base fashion that is read from the position of DNA fragments in polyacrylamide gels where the fragments are produced by base specific chemical degradation or chain termination techniques.

To date, however, SBH protocols are not generally useful for sequencing long regions of nucleic acid. In order for SBH protocols to become useful sequencing technology, suitable solutions to several limiting factors must be identified. For example, in SBH protocols, oligonucleotide arrays expand exponentially in size as a function of the length of the oligonucleotide probe. Although oligonucleotide arrays constructed of probes nine nucleotides in length are routinely synthesized (Gunderson et al., 1998) arrays of longer probes become increasingly difficult to contain within a workable area. The method of contiguous stacking (Khrapko et al., 1989) somewhat alleviates this problem by hybridizing the target fragment to an array of p-mers in the presence of a complete set of q-mers, effectively producing the same results as if an array of (p+q)-mers had been used. Typical values of p and q are eight and five respectively, resulting in an effective probe length of thirteen. Gapped probes, containing one or more universal bases, have also been used to increase effective probe length and to provide an effective solution to the problem of exponentially increasing array size (Bains, 1991). A further impediment to the development of the industrial application of SBH is the identification of hybridization conditions and protocols that are optimal for hybridization of all probe sequences. Fluorescence signal intensity of individual SBH array addresses will be dependent on the intensity of probe hybridization occurring at each address (Drmanac, 2000). Thus, the intensity of hybridization to an SBH array is not quantitative and cannot be used to estimate the number of occurrences of any particular sequence in the target. Also, both ‘false-positive’ and ‘false-negative’ hybridizations can occur. However, this problem can usually be overcome by the use of a particular reconstruction algorithm.

Primarily, the industrial application of SBH is inhibited because two or more nucleic acid sequences will very often have identical hybridization spectra when hybridized to an oligonucleotide array, making it impossible for an alignment algorithm to select and align the correct overlapping sequence. Under these circumstances, the alignment algorithm is only able to align a set of subsequences until a sequence ambiguity is encountered, at which point no further assembly of the sequence from hybridization spectra can continue and the complete sequence cannot be de-convoluted further. Thus identical SBH hybridization spectra create instances of sequence ambiguity when reconstructing an unknown nucleotide sequence. For example, when probed with an array of 4-mer oligonucleotide probes, the DNA fragments actacctatctag and actatctacctag will have identical SBH spectra (acta, ctac, tacc, acct, ccta, ctat, tatc, atct, tcta, ctag). Consequently, the original sequence cannot be inferred from the SBH spectrum. A mathematical analysis of this phenomenon shows that when probes of length p are used, a sequence ambiguity will arise if a repeated subsequence of length p−1 is present in the target fragment (note that the 3-mer cta is repeated in the above example).

From the viewpoint of sequence assembly, it is clear that reconstruction of a target sequence will be interrupted wherever a given overlapping (p−1)-mer is repeated two or more times because any of the two or more (p−1)-mers differing in the last nucleotide can be used in extending the sequence. This branching point limits unambiguous assembly of the target sequence. Multiple occurrences of (p−1)-mers are, therefore, the cause of interruption of ordering the overlap of p-mers in the process of sequence assembly. This interruption leads to a sequence remaining in the form of sub-fragments. Thus, the unambiguous segments between branching points whose order cannot be uniquely determined are termed sequence sub-fragments. The sum of all sub-fragments is longer than the actual target sequence because of overlapping short ends. Generally, sub-fragments cannot be assembled in a linear order without additional information since they have shared (p−1)-mers at their ends and starts. Different numbers of sub-fragments are obtained for each target sequence depending on the number of its repeated (p−1)-mers. The number depends on the value of p−1 and the length of the target. For fixed probe length p, the likelihood of unambiguous reconstruction rapidly decreases as the length of target fragments increases.

Accordingly, the problem of ambiguities in reconstruction of a target sequence seriously limits the application of SBH. A recent study by the inventors has shown that the length of DNA fragment that can be reliably sequenced using current SBH technology is even shorter than was previously estimated. For example, to achieve a 95% success rate in unambiguous reconstruction using standard probes of length 8, 9 or 10, DNA fragments must be no longer than 25, 40, and 50 bases, respectively. To achieve the same efficiency for fragments of length 100 bases, the minimum probe length required is 13. In contrast, gel-based sequencing techniques routinely sequence fragments 500 to 1000 bases in length.

Initial studies by the present inventors focused on developing methods for ameliorating difficulties in nucleic acid sequence analysis caused by particular local sequence characteristics. In this connection, some nucleic acid regions interfere with vector or host functions (Bieth et al., 1997; Williamson et al., 1993) and are unclonable. Other regions throw off the sequencing enzymes because of an unusually high or low GC content (Perng et al., 1994). Still others contain a number of direct or inverted repeats (The Sanger Centre 1998; Chissoe et al., 1997). Direct repeats cause problems for polymerase chain reaction (PCR) because priming sites must lie in single copy sequence, otherwise the PCR will amplify several regions of sequence simultaneously. It can, therefore, be difficult to identify suitable priming sites in a region containing direct repeats. Inverted repeats can cause problems because they lead to base pairing between different regions of a single stranded DNA molecule. The presence of inverted repeats has been identified as a significant cause of poor sub-clone coverage (Chissoe et al., 1997). Particular classes of DNA encountered in the human sequencing project are refractory to cloning and/or sequencing and typically comprise highly repetitive sequences such as LINES (0.7-7 kb) and SINES (0.3 kb) and centromeric and telomeric regions spanning many hundreds of kilobases (The Sanger Centre 1998; Weber & Myers 1997).

In work leading up to the present invention, the inventors developed a novel strategy for sequencing a target nucleic acid molecule. The strategy involves providing a plurality of variants whose sequences are distinguished individually from the target nucleic acid molecule by the addition, deletion and/or substitution of at least one nucleotide and analyzing the individual sequences of the variants and optionally one or more sequences derived from or adjacent to the target nucleic acid molecule to infer or otherwise deduce at least a portion of the sequence of the target molecule. In one example of this analysis, the sequences are compared to provide a consensus sequence corresponding to all or part of the target molecule. This strategy is suitable for a variety of applications, including the analysis of molecules whose local sequence characteristics render them refractory to sequence analysis. For example, this strategy has been adapted to SBH, which dramatically improves its industrial applicability. In one example of this application, at least one secondary nucleic acid sequence is produced that varies from an unknown primary nucleic acid sequence by the addition, deletion and/or substitution of at least one nucleotide such that at least one copy of a subsequence, which is repeated in the primary or target nucleic acid sequence, is altered or destroyed in the secondary nucleic acid sequence. The secondary nucleic acid sequence thus provides additional sequence information that can be used to resolve a sequence ambiguity and to permit the reconstruction of a nucleotide sequence from a target nucleic acid molecule.

These novel strategies have been reduced to practice in methods for analyzing multi-subunit sequences and for resolving ambiguities in sequence analysis, as described hereinafter.

### SUMMARY OF THE INVENTION

Accordingly, in one aspect, the present invention broadly resides in a method for analyzing a primary subunit sequence, comprising:

providing at least one secondary subunit sequence which varies from said primary subunit sequence by the addition, deletion and/or substitution of at least one subunit; and

analyzing the variation in said at least one secondary subunit sequence to infer information relative to the primary subunit sequence.

The information that is inferred preferably relates to a property or feature or physical parameter of the primary subunit sequence, including but not restricted to, its sequence information, structure, size or a refractory behavior to the execution of a task thereon (e.g., cloning or sequencing).

The analysis may optionally use information derived from the primary subunit sequence or from a sequence adjacent thereto.

In another aspect, the invention provides a method for analyzing the refractory behavior of a primary subunit sequence to the execution of a task thereon, comprising:

providing at least one secondary subunit sequence which varies from said primary subunit sequence by the addition, deletion and/or substitution of at least one subunit; and

determining whether said variation is associated with the abrogation, inhibition or otherwise amelioration of said refractory behavior to render said task wholly or partially executable on said at least one secondary subunit sequence.

In yet another aspect, the invention provides a method for analyzing the refractory behavior of a primary subunit sequence to the execution of a task thereon, comprising:

providing at least one secondary subunit sequence which varies from said primary subunit sequence by the addition, deletion and/or substitution of at least one subunit, wherein said variation is associated with the abrogation, inhibition or otherwise amelioration of said refractory behavior; and

determining the effects of wholly or partially executing the task on said at least one secondary subunit sequence.

In still yet another aspect, the invention provides a method for wholly or partially executing, in effect, a task on a primary subunit sequence which is refractory to the execution of said task, comprising:

providing at least one secondary subunit sequence which varies from said primary subunit sequence by the addition, deletion and/or substitution of at least one subunit, wherein said variation is associated with the abrogation, inhibition or otherwise amelioration of said refractory behavior;

executing said task, in whole or in part, on said at least one secondary subunit sequence;

determining the effects of wholly or partially executing said task on said at least one secondary subunit sequence; and

inferring some or all of the likely effects of executing said task on said primary subunit sequence based on the effects of wholly or partially executing said task on said at least one secondary subunit sequence and optionally based on information derived from the primary subunit sequence or from a sequence adjacent thereto.

In still another aspect, the invention envisions a method for wholly or partially executing, in effect, a task to which a primary nucleic acid sequence is refractory, comprising:

providing at least one secondary nucleic acid sequence which varies from said primary nucleic acid sequence, or complement thereof, by the addition, deletion and/or substitution of at least one nucleotide, wherein said variation is associated with the abrogation, inhibition or otherwise amelioration of said refractory behavior;

executing said task, in whole or in part, on said at least one secondary nucleic acid sequence;

determining the effects of wholly or partially executing said task on said at least one secondary nucleic acid sequence; and

inferring some or all of the likely effects of executing said task on said primary nucleic acid sequence based on the effects of wholly or partially executing said task on said at least one secondary nucleic acid sequence and optionally based on information derived from the primary nucleic acid sequence or from a sequence adjacent thereto.

In a preferred embodiment, the task is selected from sequence analysis or cloning.

In a further aspect, the invention contemplates a method for wholly or partially executing, in effect, a task to which a primary amino acid sequence is refractory, comprising:

providing at least one secondary amino acid sequence which varies from said primary amino acid sequence by the addition, deletion and/or substitution of at least one amino acid residue, wherein said variation is associated with the abrogation, inhibition or otherwise amelioration of said refractory behavior;

executing said task, in whole or in part, on said at least one secondary amino acid sequence;

determining the effects of wholly or partially executing said task on said at least one secondary amino acid sequence; and

inferring some or all of the likely effects of executing said task on said primary amino acid sequence based on the effects of wholly or partially executing said task on said at least one secondary amino acid sequence and optionally based on information derived from the primary amino acid sequence or from a sequence adjacent thereto.

In still a further aspect, the invention provides a method for wholly or partially deducing the sequence of a target subunit sequence, comprising:

providing the sequences of a plurality of variants, which are distinguished individually from the target subunit sequence by the addition, deletion and/or substitution of at least one subunit;

comparing the individual sequences of said variants with each other and optionally with a sequence derived from the target subunit sequence or from a sequence adjacent thereto to deduce a consensus sequence, which corresponds to all or part of the target subunit sequence.

The comparison may be effected using any suitable technique that compares sequence information to thereby deduce a consensus sequence. Such techniques include, but are not restricted to, sequence alignment or probabilistic techniques as for example described herein.

In one embodiment, this method can be used advantageously to deduce the sequence of at least a portion of a target subunit sequence that is refractory to sequence analysis.

Accordingly, in still another aspect, the invention provides a method for wholly or partially deducing the sequence of a target subunit sequence which is refractory to sequence analysis, comprising:

providing the sequences of a plurality of variants, which are distinguished individually from the target subunit sequence by the addition, deletion and/or substitution of at least one subunit, wherein said variation is associated with the abrogation, inhibition or otherwise amelioration of said refractory behavior;

comparing the individual sequences of said variants with each other and optionally with a sequence derived from the target subunit sequence or from a sequence adjacent thereto to deduce a consensus sequence, which corresponds to all or part of the target subunit sequence.

In another aspect, the invention encompasses a method for wholly or partially deducing the sequence of a target subunit sequence which is refractory to sequence analysis, comprising:

providing a plurality of variants whose sequences are distinguished individually from the target subunit sequence by the addition, deletion and/or substitution of at least one subunit, wherein said variation is associated with the abrogation, inhibition or otherwise amelioration of said refractory behavior;

sequencing said variants, in whole or in part, to provide a sequence for each variant; and

comparing the individual sequences of said variants with each other and optionally with a sequence derived from the target subunit sequence or from a sequence adjacent thereto to deduce a consensus sequence, which corresponds to all or part of the target subunit sequence.

In yet another aspect, the invention encompasses a method for wholly or partially deducing the sequence of a target nucleic acid sequence which is refractory to sequence analysis, comprising:

providing a plurality of variants whose sequences are distinguished individually from the target nucleic acid sequence, or complement thereof, by the addition, deletion and/or substitution of at least one nucleotide, wherein said variation is associated with the abrogation, inhibition or otherwise amelioration of said refractory behavior;

sequencing said variants, in whole or in part, to provide a sequence for each variant; and

comparing the individual sequences of said variants, or complements thereof, with each other and optionally with a sequence derived from the target nucleic acid sequence, or complement thereof, or from a sequence adjacent to the target nucleic acid sequence, or complement thereof, to deduce a consensus sequence, which corresponds to all or part of the target nucleic acid sequence.

In yet another aspect, the invention contemplates a method for wholly or partially deducing the sequence of a target amino acid sequence which is refractory to sequence analysis, comprising:

providing a plurality of variants whose sequences are distinguished individually from the target amino acid sequence by the addition, deletion and/or substitution of at least one amino acid residue, wherein said variation is associated with the abrogation, inhibition or otherwise amelioration of said refractory behavior;

sequencing said variants, in whole or in part, to provide a sequence for each variant; and

comparing the individual sequences of said variants with each other and optionally with a sequence derived from the target amino acid sequence or from a sequence adjacent thereto to deduce a consensus sequence, which corresponds to all or part of the target amino acid sequence.

According to another aspect of the invention, there is provided a method for determining the sequence of a primary subunit sequence, said method comprising:

providing at least one secondary subunit sequence which varies from said primary sequence by the addition, deletion and/or substitution of at least one subunit; and

comparing overlapping subsequences corresponding to said at least one secondary subunit sequence and to said primary subunit sequence to reconstruct at least a portion of the primary subunit sequence.

Preferably, the method comprises alternately reconstructing said primary subunit sequence and said at least one secondary subunit sequence using an end portion of a respective reconstruction as a guide to extend another reconstruction.

In a preferred embodiment of this type, the alternate reconstruction comprises:

comparing a portion of the primary subunit sequence with subsequences corresponding to said at least one secondary subunit sequence to identify a subsequence which aligns best with said portion and which extends unambiguously in said alignment a reconstruction of said at least one secondary subunit sequence beyond said portion; and

comparing an end portion of said reconstruction with subsequences corresponding to said primary subunit sequence to identify a subsequence which aligns best with said end portion of said reconstruction and which extends unambiguously in said alignment the reconstruction of said primary subunit sequence.

In an alternate embodiment, the alternate reconstruction preferably comprises deducing a best alignment between a subsequence and a sequence reconstruction by comparing the alignment of different subsequences with said reconstruction to produce a plurality of extended reconstructions together with individual alignment scores for each reconstruction, and optionally iteratively comparing downstream alignments of extended reconstructions using subsequences available for reconstruction, and determining a reconstruction with the highest scoring alignment to thereby deduce said best alignment.

The above method is particularly, but not exclusively, useful for shotgun sequencing and SBH techniques. In one embodiment, this method can be used to extend an reconstruction of a primary subunit sequence, which reconstruction is incompletable due to the presence of repeated subsequences in said primary subunit sequence.

Accordingly, in—another aspect of the invention, there is provided a method for unambiguously extending an incomplete reconstruction of a primary subunit sequence by comparing overlapping subsequences corresponding to said primary subunit sequence, wherein said reconstruction is incompletable due to the presence of repeated subsequences in said primary subunit sequence, said method comprising:

providing at least one secondary subunit sequence which varies from said primary sequence by the addition, deletion and/or substitution of at least one subunit, wherein said variation is associated with the alteration or destruction of at least one of said repeated subsequences; and

comparing overlapping subsequences corresponding to said at least one secondary subunit sequence and to said primary subunit sequence, to unambiguously extend said incomplete reconstruction.

Preferably, some or all of the subsequences of said at least one secondary subunit sequence, which are varied relative to the repeated subsequences, are different relative to each other.

Suitably, the method comprises comparing an end portion of said incomplete reconstruction with one or more subsequences corresponding to said at least one secondary subunit sequence to deduce an unambiguous extension to said incomplete reconstruction.

Preferably, the method comprises alternately reconstructing said primary subunit sequence and said at least one secondary subunit sequence using an end portion of a respective reconstruction as a guide to extend another reconstruction.

In a preferred embodiment of this type, the method comprises:

comparing an end portion of said incomplete reconstruction with subsequences corresponding to said at least one secondary subunit sequence to identify a subsequence which aligns best with said end portion and which extends unambiguously in said alignment a reconstruction of said at least one secondary subunit sequence beyond the incomplete reconstruction of said primary subunit sequence to form an extended reconstruction of said at least one secondary subunit sequence; and

comparing an end portion of said extended reconstruction with subsequences corresponding to said primary subunit sequence to identify a subsequence which aligns best with said end portion of said extended reconstruction and which extends unambiguously in said alignment the incomplete reconstruction of said primary subunit sequence to form an extended reconstruction of said primary subunit sequence.

In an alternate embodiment, the method preferably comprises deducing a best alignment between a subsequence and an incomplete reconstruction by comparing the alignment of different subsequences with said incomplete reconstruction to produce a plurality of extended reconstructions together with individual alignment scores for each reconstruction, and optionally iteratively comparing downstream alignments of extended reconstructions using subsequences available for reconstruction, and determining a reconstruction with the highest scoring alignment to thereby deduce said best alignment.

In a further aspect, the invention features a method of forming an extension to an incomplete tiling path of overlapping subsequences corresponding to a primary target subunit sequence comprising repeated subsequences, said method comprising:

providing at least one secondary subunit sequence which varies from said primary sequence by the addition, deletion and/or substitution of at least one subunit, wherein said variation is associated with the alteration or destruction of at least one of said repeated subsequences; and

comparing overlapping subsequences corresponding to said at least one secondary subunit sequence and to said primary subunit sequence to extend said incomplete tiling path.

In another aspect, the invention features a method for unambiguously extending an incomplete reconstruction of a primary subunit sequence by comparing overlapping subsequences, of length p, corresponding to said primary subunit sequence, wherein said reconstruction is incompletable due to the presence of repeated subsequences, of length p−1, in said primary subunit sequence, said method comprising:

providing at least one secondary subunit sequence which varies from said primary sequence by the addition, deletion and/or substitution of at least one subunit, wherein said variation is associated with the alteration or destruction of at least one of said repeated subsequences; and

comparing overlapping subsequences, of length p, corresponding to said at least one secondary subunit sequence and to said primary subunit sequence, to unambiguously extend said incomplete reconstruction.

In yet another aspect, the invention contemplates a method for unambiguously extending an incomplete reconstruction of a primary nucleic acid sequence by comparing overlapping subsequences, of length p, corresponding to said primary nucleic acid sequence, wherein said reconstruction is incompletable due to the presence of repeated subsequences of length p−1 in said primary nucleic acid sequence, said method comprising:

providing at least one secondary nucleic acid sequence which varies from said primary sequence, or complement thereof, by the addition, deletion and/or substitution of at least one nucleotide, wherein said variation is associated with the alteration or destruction of at least one of said repeated subsequences, or complement thereof; and

comparing overlapping subsequences, of length p, corresponding to said at least one secondary nucleic acid sequence and to said primary nucleic acid sequence, or complement thereof, to unambiguously extend said incomplete reconstruction.

Suitably, the method further comprises generating said subsequences using a sequence analysis technique.

In a preferred embodiment, the sequence analysis technique is a Sequencing by Hybridization (SBH) technique or a shotgun sequencing technique.

In still a further aspect, the invention envisions a method for unambiguously extending an incomplete reconstruction of a primary amino acid sequence by comparing overlapping subsequences, of length p, corresponding to said primary amino acid sequence, wherein said reconstruction is incompletable due to the presence of repeated subsequences of length p−1 in said primary amino acid sequence, said method comprising:

providing at least one secondary amino acid sequence which varies from said primary sequence by the addition, deletion and/or substitution of at least one amino acid residue, wherein said variation is associated with the alteration or destruction of at least one of said repeated subsequences; and

comparing overlapping subsequences, of length p, corresponding to said at least one secondary amino acid sequence and to said primary amino acid sequence, to unambiguously extend said incomplete reconstruction.

In another aspect, the invention features a method of forming an extension to an incomplete tiling path of overlapping subsequences, of length p, corresponding to a primary target subunit sequence comprising repeated subsequences of length p−1, said method comprising:

providing at least one secondary subunit sequence which varies from said primary sequence by the addition, deletion and/or substitution of at least one subunit, wherein said variation is associated with the alteration or destruction of at least one of said repeated subsequences; and

comparing overlapping subsequences, of length p, corresponding to said at least one secondary subunit sequence and to said primary subunit sequence to extend said incomplete tiling path.

Preferably, the at least one secondary subunit sequence is produced by mutagenesis of the primary subunit sequence.

Suitably, a secondary subunit sequence is produced by mutagenesis of another secondary subunit sequence.

In a preferred embodiment, a parent subunit sequence is mutagenized to produce at least one variant subunit sequence in which at least 5%, preferably at least 10%, more preferably at least 20%, even more preferably at least 30%, and still even more preferably at least 40% of subunits are different relative to the parent subunit sequence.

Preferably, the subunit sequences are nucleic acid sequences. In a preferred embodiment of this type, a parent nucleic acid sequence is mutagenized by incorporation of nucleotide analogues, which are preferably, but not exclusively, selected from dPTP (6-(2-deoxy-B-D-ribofuranosyl)-3,4-dihydro-8H-pyrimido-[4,5-C]oxazin-7-one triphosphate) or 8-oxo-dGTP (8-oxo-deoxyguanosine triphosphate) as for example described in U.S. Pat. No. 6,153,745 and U.S. Pat. No. 6,313,286. In another preferred embodiment of this type, a parent nucleic acid sequence is mutagenized using low fidelity nucleic acid amplification reaction and an error prone DNA polymerase, which is preferably thermostable. In yet another preferred embodiment of this type, a parent nucleic acid sequence is mutagenized using a repair deficient host, which is preferably a bacterium.

In yet another aspect, the invention encompasses a whole or partial primary subunit sequence or a whole or partial secondary subunit sequence obtained by the method as broadly described above.

In still another aspect, the invention provides a method for wholly or partially determining a primary nucleic acid sequence, comprising:

providing a set of overlapping subsequences corresponding to said primary nucleic acid sequence, or complement thereof;

generating hybridization data by exposing an array of oligonucleotide probes, under stringent hybridization conditions, to at least one secondary nucleic acid sequence which varies from said primary nucleic acid sequence, or complement thereof, by the addition, deletion and/or substitution of at least one nucleotide;

processing the hybridization data to detect which of said probes have hybridized to said at least one secondary nucleic acid sequence to thereby determine a set of subsequences corresponding to said at least one secondary nucleic acid sequence; and

comparing overlapping subsequences corresponding to said at least one secondary nucleic acid sequence and to said primary nucleic acid sequence, or complement thereof, to wholly or partially determine said primary nucleic acid sequence.

In a preferred embodiment, the method further comprises:

generating other hybridization data by exposing an array of oligonucleotide probes to said primary nucleic acid sequence, or complement thereof, under stringent hybridization conditions; and

processing said other hybridization data to detect which of said probes have hybridized to said primary nucleic acid sequence, or complement thereof, to thereby determine a set of subsequences corresponding to said primary nucleic acid sequence, or complement thereof.

The above method is particularly, but not exclusively, useful for determining a primary nucleic acid sequence that comprises repeated subsequences of length p−1. Thus, in a related aspect, the invention contemplates a method for wholly or partially determining a primary nucleic acid sequence comprising repeated subsequences of length p−1, comprising:

providing a set of overlapping subsequences, of length p, corresponding to said primary nucleic acid sequence, or complement thereof;

generating hybridization data by exposing an array of oligonucleotide probes comprising a sequence of length p, under stringent hybridization conditions, to at least one secondary nucleic acid sequence which varies from said primary nucleic acid sequence, or complement thereof, by the addition, deletion and/or substitution of at least one nucleotide, wherein said variation is associated with the alteration or destruction of at least one of said repeated subsequences, or complement thereof;

processing the hybridization data to detect which of said probes have hybridized to said at least one secondary nucleic acid sequence to thereby determine a set of subsequences, of length p, corresponding to said at least one secondary nucleic acid sequence; and

comparing overlapping subsequences, of length p, corresponding to said at least one secondary nucleic acid sequence and to said primary nucleic acid sequence, or complement thereof, to wholly or partially determine said primary nucleic acid sequence.

In a preferred embodiment, the method further comprises:

generating other hybridization data by exposing an array of oligonucleotide probes comprising a sequence of length p, to said primary nucleic acid sequence, or complement thereof, under stringent hybridization conditions; and

processing said other hybridization data to detect which of said probes have hybridized to said primary nucleic acid sequence, or complement thereof, to thereby determine a set of subsequences, of length p, corresponding to said primary nucleic acid sequence, or complement thereof.

In another preferred embodiment, the step of processing is performed by a processing system.

In yet another preferred embodiment, the step of comparing is performed by a processing system.

According to another aspect, the invention provides a method for extending an incomplete reconstruction of a primary nucleic acid sequence by comparing overlapping subsequences, of length p, corresponding to said primary nucleic acid sequence, wherein said reconstruction is incompletable due to the presence of repeated subsequences of length p−1 in said primary nucleic acid sequence, said method comprising:

exposing an array of oligonucleotide probes comprising a sequence of length p, under stringent hybridization conditions, to at least one secondary nucleic acid sequence which varies from said primary nucleic acid sequence, or complement thereof, by the addition, deletion and/or substitution of at least one nucleotide, wherein said variation is associated with the alteration or destruction of at least one of said repeated subsequences, or complement thereof;

processing the hybridization data to detect which of said probes have hybridized to said at least one secondary nucleic acid sequence to thereby determine a set of subsequences, of length p, corresponding to said at least one secondary nucleic acid sequence; and

comparing overlapping subsequences, of length p, corresponding to said at least one secondary nucleic acid sequence and to said primary nucleic acid sequence, or complement thereof, to unambiguously extend said incomplete reconstruction.

In another aspect, the invention provides a computer program product for wholly or partially deducing the sequence of a target subunit sequence, said computer program product including computer executable code which when implemented on a suitable processing system causes the processing system to:

receive the sequences of a plurality of variants, which are distinguished individually from the target subunit sequence by the addition, deletion and/or substitution of at least one subunit;

optionally receive a sequence derived from the target subunit sequence or from a sequence adjacent thereto;

compare the individual sequences of said variants with each other and optionally with the sequence derived from the target subunit sequence or with the adjacent sequence to deduce a consensus sequence, which corresponds to all or part of the target subunit sequence.

In yet another aspect, the invention provides a processing system for wholly or partially deducing the sequence of a target subunit sequence, said processing system being adapted to:

receive data representing the sequences of a plurality of variants, which are distinguished individually from the target subunit sequence by the addition, deletion and/or substitution of at least one subunit, and optionally representing a sequence that is derived from the target subunit sequence or from a sequence adjacent thereto; and

compare the individual sequences of said variants with each other and optionally with the sequence derived from the target subunit sequence or with the adjacent sequence to deduce a consensus sequence, which corresponds to all or part of the target subunit sequence.

In yet another aspect, the invention provides a computer program product for wholly or partially deducing the sequence of a target subunit sequence which is refractory to sequence analysis, said computer program product including computer executable code which when implemented on a suitable processing system causes the processing system to:

receive the sequences of a plurality of variants, which are distinguished individually from the target subunit sequence by the addition, deletion and/or substitution of at least one subunit;

optionally receive a sequence derived from the target subunit sequence or from a sequence adjacent thereto;

compare the individual sequences of said variants with each other and optionally with the sequence derived from the target subunit sequence or with the adjacent sequence to deduce a consensus sequence, which corresponds to all or part of the target subunit sequence.

In still yet another aspect, the invention provides a processing system for wholly or partially deducing the sequence of a target subunit sequence, said processing system being adapted to

receive data representing the sequences of a plurality of variants, which are distinguished individually from the target subunit sequence by the addition, deletion and/or substitution of at least one subunit, and optionally representing a sequence that is derived from the target subunit sequence or from a sequence adjacent thereto; and

compare the individual sequences of said variants with each other and optionally with the sequence derived from the target subunit sequence or with the adjacent sequence to deduce a consensus sequence, which corresponds to all or part of the target subunit sequence.

In a preferred embodiment, the processing system further comprises a store for storing said data.

In another preferred embodiment, the processing system is further adapted to generate an indication of the target subunit sequence. In an especially preferred embodiment of this type, the processing system comprises a display, which displays said indication.

Suitably, the subunit sequences are selected from nucleic acid sequences or amino acid sequences.

In another aspect, the invention provides a computer program product for wholly or partially deducing the sequence of a primary subunit sequence, said computer program product including computer executable code which when implemented on a suitable processing system causes the processing system to:

receive the sequences of a plurality of variants, which are distinguished individually from the target subunit sequence by the addition, deletion and/or substitution of at least one subunit;

optionally receive a sequence derived from the primary subunit sequence or from a sequence adjacent thereto;

compare overlapping sequences corresponding to said variants and optionally to the sequence derived from, or adjacent to, the primary subunit sequence to reconstruct at least a portion of the primary subunit sequence.

Preferably, the computer executable code which when implemented on said processing system causes the processing system to alternately reconstruct said primary subunit sequence and said at least one secondary subunit sequence using an end portion of a respective reconstruction as a guide to extend another reconstruction.

In a preferred embodiment of this type, the computer executable code which when implemented on said processing system causes the processing system to:

compare a portion of the primary subunit sequence with subsequences corresponding to said at least one secondary subunit sequence to identify a subsequence which aligns best with said portion and which extends unambiguously in said alignment a reconstruction of said at least one secondary subunit sequence beyond said portion; and

compare an end portion of said reconstruction with subsequences corresponding to said primary subunit sequence to identify a subsequence which aligns best with said end portion of said reconstruction and which extends unambiguously in said alignment the reconstruction of said primary subunit sequence.

In a preferred embodiment of this type, the computer executable code which when implemented on said processing system causes the processing system to deduce a best alignment between a subsequence and a sequence reconstruction by comparing the alignment of different subsequences with said reconstruction to produce a plurality of extended reconstructions together with individual alignment scores for each reconstruction, and optionally iteratively comparing downstream alignments of extended reconstructions using subsequences available for reconstruction, and determining a reconstruction with the highest scoring alignment to thereby deduce said best alignment.

In still yet another aspect, the invention provides a processing system for determining the sequence of a primary subunit sequence, said processing system being adapted to:

receive data representing at least one secondary subunit sequence which varies from said primary subunit sequence by the addition, deletion and/or substitution of at least one subunit;

compare overlapping subsequences corresponding to said at least one secondary subunit sequence and to said primary subunit sequence to reconstruct at least a portion of the primary subunit sequence.

In another aspect, the invention contemplates a computer program product for unambiguously extending an incomplete reconstruction of a primary subunit sequence by comparing overlapping subsequences corresponding to said primary subunit sequence, wherein said reconstruction is incompletable due to the presence of repeated subsequences in said primary subunit sequence, said computer program product including computer executable code which when implemented on a suitable processing system causes the processing system to:

receive data representing subsequences corresponding to said primary subunit sequence;

receive data representing subsequences corresponding to a plurality of variants whose sequences are distinguished individually from the target subunit sequence by the addition, deletion and/or substitution of at least one subunit, wherein said variation is associated with the alteration or destruction of at least one of said repeated subsequences; and

compare overlapping subsequences corresponding to said at least one secondary subunit sequence and to said primary subunit sequence, to unambiguously extend said incomplete reconstruction and to thereby form an extended reconstruction.

Preferably, the computer executable code which when implemented on said processing system causes the processing system to alternately reconstruct said primary subunit sequence and said at least one secondary subunit sequence using an end portion of a respective reconstruction as a guide to extend another reconstruction.

In a preferred embodiment of this type, the computer executable code which when implemented on said processing system causes the processing system to:

compare an end portion of said incomplete reconstruction with subsequences corresponding to said at least one secondary subunit sequence to identify a subsequence which aligns best with said end portion and which extends unambiguously in said alignment a reconstruction of said at least one secondary subunit sequence beyond the incomplete reconstruction of said primary subunit sequence to form an extended reconstruction of said at least one secondary subunit sequence; and

compare an end portion of said extended reconstruction with subsequences corresponding to said primary subunit sequence to identify a subsequence which aligns best with said end portion of said extended reconstruction and which extends unambiguously in said alignment the incomplete reconstruction of said primary subunit sequence to form an extended reconstruction of said primary subunit sequence.

In a preferred embodiment, said subunit sequences are selected from nucleic acid sequences or amino acid sequences.

In still yet another aspect, the invention provides a processing system for unambiguously extending an incomplete reconstruction of a primary subunit sequence by comparing overlapping subsequences corresponding to said primary subunit sequence, wherein said reconstruction is incompletable due to the presence of repeated subsequences in said primary subunit sequence, said processing system being adapted to:

receive data representing at least one secondary subunit sequence which varies from said primary subunit sequence by the addition, deletion and/or substitution of at least one subunit, wherein said variation is associated with the alteration or destruction of at least one of said repeated subsequences;

compare overlapping subsequences corresponding to said at least one secondary subunit sequence and to said primary subunit sequence, to unambiguously extend said incomplete reconstruction and to thereby form an extended reconstruction.

In yet another aspect, the invention resides in a computer program product for determining at least a portion of a primary nucleic acid sequence, said computer program product including computer executable code which when implemented on a suitable processing system causes the processing system to:

receive data representing a set of overlapping subsequences corresponding to said primary nucleic acid sequence, or complement thereof;

receive features of an oligonucleotide array whose probes detect specifically individual target oligonucleotide sequences under stringent hybridization conditions;

receive hybridization data from hybridization reactions between the oligonucleotide probes in the array and at least one secondary nucleic acid sequence which varies from said primary nucleic acid sequence, or complement thereof, by the addition, deletion and/or substitution of at least one nucleotide;

process the hybridization data to detect which of said probes have hybridized to said at least one secondary nucleic acid sequence to thereby determine a set of subsequences corresponding to said at least one secondary nucleic acid sequence; and

compare overlapping subsequences corresponding to said at least one secondary nucleic acid sequence and to said primary nucleic acid sequence, or complement thereof, to wholly or partially determine said primary nucleic acid sequence.

In yet another aspect, the invention resides in a computer program product for unambiguously extending an incomplete reconstruction of a primary subunit sequence by comparing overlapping subsequences, of length p, corresponding to said primary subunit sequence, wherein said reconstruction is incompletable due to the presence of repeated subsequences, of length p−1, in said primary subunit sequence, said computer program product including computer executable code which when implemented on a suitable processing system causes the processing system to:

receive data representing a set of overlapping subsequences, of length p, corresponding to a primary nucleic acid sequence, or to a complement thereof, comprising repeated subsequences of length p−1;

receive features of an oligonucleotide array whose probes detect specifically individual target oligonucleotide sequences under stringent hybridization conditions;

receive hybridization data from hybridization reactions between the oligonucleotide probes in the array and at least one secondary nucleic acid sequence which varies from said primary nucleic acid sequence, or complement thereof, by the addition, deletion and/or substitution of at least one nucleotide, wherein said variation is associated with the alteration or destruction of at least one of said repeated subsequences;

process the hybridization data to determine which of said target oligonucleotide sequences are contained in said at least one secondary nucleic acid sequence to determine a set of subsequences, of length p, corresponding to said at least one secondary nucleic acid sequence; and

compare overlapping subsequences, of length p, from both sets, to unambiguously extend said incomplete reconstruction.

In a preferred embodiment of this type, the computer program product comprises computer executable code which when implemented on said processing system causes the processing system to:

alternately reconstruct said primary nucleic acid sequence, or complement thereof, and said at least one secondary nucleic acid sequence using an end portion of a respective reconstruction as a guide to extend another reconstruction.

In an especially preferred embodiment, the computer program product comprises computer executable code which when implemented on said processing system causes the processing system to:

compare an end portion of an incomplete reconstruction of said primary nucleic acid sequence, or complement thereof, with subsequences corresponding to said at least one secondary nucleic acid sequence to identify a subsequence which aligns best with said end portion and which extends unambiguously in said alignment a reconstruction of said at least one secondary nucleic acid sequence beyond the incomplete reconstruction of said primary nucleic acid sequence, or complement thereof, to form an extended reconstruction of said at least one secondary nucleic acid sequence; and

compare an end portion of said extended reconstruction with subsequences corresponding to said primary nucleic acid sequence, or complement thereof, to identify a subsequence which aligns best with said end portion of said extended reconstruction and which extends unambiguously in said alignment the incomplete reconstruction of said primary nucleic acid sequence, or complement thereof, to form an extended reconstruction of said primary nucleic acid sequence, or complement thereof.

In another preferred embodiment, the computer program product comprises computer executable code which when implemented on said processing system causes the processing system to:

receive hybridization data from hybridization reactions between the oligonucleotide probes in the array and said primary nucleic acid sequence, or complement thereof; and

process the hybridization data to determine which of said target oligonucleotide sequences are hybridized to said primary nucleic acid sequence, or complement thereof, to determine a set of subsequences, of length p, corresponding to said primary nucleic acid sequence, or to said complement.

Preferably, said computer program product comprises computer executable code which when implemented on said processing system causes the processing system to receive the sequence of an oligonucleotide probe in each feature of the oligonucleotide array.

According to another aspect, the invention envisions a method for identifying an error in a target subunit sequence, comprising:

providing a plurality of variants whose sequences are distinguished individually from said target subunit sequence by the addition, deletion and/or substitution of at least one subunit; and

aligning the individual sequences of said variants to each other and optionally to said target subunit sequence to deduce a consensus sequence, wherein variation from said consensus sequence by said target subunit sequence is indicative of said error.

In yet another aspect, the invention encompasses a method for verifying the presence of a polymorphic site in a first nucleic acid sequence and in a second nucleic acid sequence, comprising:

providing a plurality of first variants whose sequences are distinguished individually from said first nucleic acid sequence by the addition, deletion and/or substitution of at least one nucleotide;

providing a plurality of second variants whose sequences are distinguished individually from said second nucleic acid sequence by the addition, deletion and/or substitution of at least one nucleotide;

aligning the individual sequences of said first variants to each other to deduce a first consensus sequence;

aligning the individual sequences of said second variants to each other to deduce a second consensus sequence; and

comparing the first and second consensus sequences, wherein a difference between said first consensus and said second consensus is indicative of the presence of a polymorphic site.

### BRIEF DESCRIPTION OF THE DRAWINGS

FIG. 1 is a flow chart broadly illustrating the steps of conventional data collection, processing and analysis.

FIG. 2 is a flow chart broadly illustrating the steps of data collection, processing and analysis using one embodiment of SAM.

FIG. 3 is a flow chart broadly illustrating the steps of data collection, processing and analysis using another embodiment of SAM.

FIG. 4 is a diagrammatic representation illustrating mutant configurations: (A) Star; (B) Path; (C) Octopus; and (D) Binary Tree.

FIG. 5 is a schematic representation of a computer system useful in the practice of the present invention.

FIG. 6 is a flow chart broadly illustrating one embodiment of the application of SAM to DNA sequencing. It is a particularization of FIG. 2.

FIG. 7 is a flow chart broadly illustrating another embodiment of the application of SAM to DNA sequencing. It is a particularization of FIG. 3.

FIG. 8 is a flow chart broadly illustrating one embodiment of the application of SAM to shotgun sequencing. It is a particularization of FIG. 6.

FIG. 9 is a flow chart broadly illustrating another embodiment of the application of SAM to shotgun sequencing. It is a particularization of FIG. 7.

FIG. 10 is a flow chart broadly illustrating another embodiment of the application of SAM to shotgun sequencing. It is a particularization of FIG. 2.

FIG. 11 is a flow chart broadly illustrating another embodiment of the application of SAM to shotgun sequencing. It is a particularization of FIG. 3.

FIG. 12 is a flow chart illustrating one embodiment of the application of SAM to SBH. It is a particularization of FIG. 2.

FIG. 13 is a flow chart illustrating another embodiment of the application of SAM to SBH. It is a particularization of FIG. 3.

FIG. 14 is a graphical representation showing the average number of errors in the reconstructed string as a function of the number of mutant copies, with different mutation probabilities.

FIG. 15 is a graphical representation showing the average number of errors in the reconstructed string as a function of the target fragment length.

FIG. 16 is a diagrammatic representation showing the secondary structure of an unmutagenized or wild-type iso-tRNA molecule specific for codon tat (Ile).

FIG. 17 is a diagrammatic representation showing the secondary structure of a high energy mutant of the iso-tRNA molecule shown in FIG. 16.

FIG. 18 is a flow chart illustrating one embodiment of the application of SAM to shotgun sequencing.

FIG. 19 is a flow chart illustrating another embodiment of the application of SAM to shotgun sequencing.

FIG. 20 is a flow chart illustrating another embodiment of the application of SAM to shotgun sequencing.

### DETAILED DESCRIPTION OF THE PREFERRED EMBODIMENT

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “complementary” refers to the topological capability or matching together of interacting surfaces of an oligonucleotide probe and its target oligonucleotide, which may be part of a larger polynucleotide. Thus, the target and its probe can be described as complementary, and furthermore, the contact surface characteristics are complementary to each other. Complementary includes base complementarity such as A is complementary to T or U, and C is complementary to G in the genetic code. However, this invention also encompasses situations in which there is non-traditional base-pairing such as Hoogsteen base pairing which has been identified in certain transfer RNA molecules and postulated to exist in a triple helix. In the context of the definition of the term “complementary”, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that hybridize less efficiently.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “contig” is meant a subunit sequence assembled from overlapping shorter sequences to form one large contiguous sequence.

The term “feature” refers to an area of a substrate having a collection of substantially same-sequence, surface immobilized oligonucleotide probes. Generally, one feature is different from another feature if the probes of the different features have substantially different nucleotide sequences. In the context of light-directed oligonucleotide synthesis, for example, a feature is a spatially addressable synthesis site as for example disclosed in U.S. Pat. Nos. 5,384,261; 5,143,854; 5,150,270; 5,593,139; 5,634,734; and WO95/11995.

By “gene” is meant a genomic nucleic acid sequence at a particular genetic locus.

By “high density polynucleotide arrays” is meant those arrays that contain at least 400 different features per cm².

The phrase “high discrimination hybridization conditions” refers to hybridization conditions in which single base mismatch may be determined.

The phrase “hybridizing specifically to” and the like refer to the binding, duplexing, or hybridizing of a molecule only to a particular nucleotide sequence under stringent conditions when that sequence is present in a complex mixture (e.g., total cellular) DNA or RNA.

By “obtained from” is meant that a sample such as, for example, a polynucleotide extract is isolated from, or derived from, a particular source of the host. For example, the extract can be obtained from a tissue or a biological fluid isolated directly from the host.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 8 to 30 nucleotides, more preferably from about 10 to 20 nucleotides and still more preferably from about 11 to 17 nucleotides, but the term can refer to molecules of any length, although the term “polynucleotide” or “nucleic acid” is typically used for large oligonucleotides. Oligonucleotides may be prepared using any suitable method, such as, for example, the phosphotriester method as described in an article by Narang et al. (1979, Methods Enzymol. 68 90) and U.S. Pat. No. 4,356,270. Alternatively, the phosphodiester method as described in Brown et al. (1979, Methods Enzymol. 68 109) may be used for such preparation. Automated embodiments of the above methods may also be used. For example, in one such automated embodiment, diethylphosphoramidites are used as starting materials and may be synthesized as described by Beaucage et al. (1981, Tetrahedron Letters 22 1859-1862). Reference also may be made to U.S. Pat. Nos. 4,458,066 and 4,500,707, which refer to methods for synthesizing oligonucleotides on a modified solid support. It is also possible to use a primer, which has been isolated from a biological source (such as a denatured strand of a restriction endonuclease digest of plasmid or phage DNA). In a preferred embodiment, the oligonucleotide is synthesized according to the method disclosed in U.S. Pat. No. 5,424,186 (Fodor et al.). This method uses lithographic techniques to synthesize a plurality of different oligonucleotides at precisely known locations on a substrate surface.

The term “oligonucleotide array” refers to a substrate having oligonucleotide probes with different known sequences deposited at discrete known locations associated with its surface. For example, the substrate can be in the form of a two dimensional substrate as described in U.S. Pat. No. 5,424,186. Such substrate may be used to synthesize two-dimensional spatially addressed oligonucleotide (matrix) arrays. Alternatively, the substrate may be characterized in that it forms a tubular array in which a two dimensional planar sheet is rolled into a three-dimensional tubular configuration. The substrate may also be in the form of a microsphere or bead connected to the surface of an optic fiber as, for example, disclosed by Chee et al. in WO 00/39587. Oligonucleotide arrays have at least two different features and a density of at least 400 features per cm². In certain embodiments, the arrays can have a density of about 500, at least one thousand, at least 10 thousand, at least 100 thousand, at least one million or at least 10 million features per cm. For example, the substrate may be silicon or glass and can have the thickness of a glass microscope slide or a glass cover slip, or may be composed of other synthetic polymers. Substrates that are transparent to light are useful when the method of performing an assay on the substrate involves optical detection. The term also refers to a probe array and the substrate to which it is attached that form part of a wafer.

The term “polynucleotide” or “nucleic acid” as used herein designates mRNA, RNA, cRNA, cDNA or DNA. The term typically refers to oligonucleotides greater than 30 nucleotides in length. Polynucleotides or nucleic acids are understood to encompass complementary strands as well as alternative backbones described herein.

The terms “polynucleotide variant” and “variant” refer to polynucleotides displaying substantial sequence identity with a reference polynucleotide sequence or polynucleotides that hybridize with a reference sequence under stringent conditions that are defined hereinafter. These terms also encompass polynucleotides in which one or more nucleotides have been added or deleted, or replaced with different nucleotides. The terms “polynucleotide variant” and “variant” also include naturally occurring allelic variants.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

The term “polypeptide variant” refers to polypeptides in which one or more amino acids have been replaced by different amino acids. It is well understood in the art that some amino acids may be changed to others with broadly similar properties without changing the nature of the activity of the polypeptide (conservative substitutions) as described hereinafter. These terms also encompass polypeptides in which one or more amino acids have been added or deleted, or replaced with different amino acids.

“Probe” refers to an oligonucleotide molecule that binds to a specific target sequence or other moiety of another nucleic acid molecule. Unless otherwise indicated, the term “probe” in the context of the present invention typically refers to an oligonucleotide probe that binds to another oligonucleotide or polynucleotide, often called the “target polynucleotide”, through complementary base pairing. Probes can bind target polynucleotides lacking complete sequence complementarity with the probe, depending on the stringency of the hybridization conditions. Oligonucleotide probes may be selected to be “substantially complementary” to a target sequence as defined herein. The exact length of the oligonucleotide probe will depend on many factors including temperature and source of probe and use of the method. For example, depending upon the complexity of the target sequence, the oligonucleotide probe may typically contain 8 to 30 nucleotides, more preferably from about 10 to 20 nucleotides and still more preferably from about 11 to 17 nucleotides capable of hybridization to a target sequence although it may contain more or fewer such nucleotides.

By “reference sequence” is meant a part or segment of a target polynucleotide that could be used to guide the selection of a target sequence.

Terms used to describe sequence relationships between two or more polynucleotides or polypeptides include “comparison window”, “sequence identity”, “percentage of sequence identity” and “substantial identity”. Because two polynucleotides may each comprise (1) a sequence (i.e., only a portion of the complete polynucleotide sequence) that is similar between the two polynucleotides, and (2) a sequence that is divergent between the two polynucleotides. Sequence comparisons between two (or more) polynucleotides are typically performed by comparing sequences of the two polynucleotides over a “comparison window” to identify and compare local regions of sequence similarity. A “comparison window” refers to a conceptual segment of at least 3 contiguous positions, usually about 5 to about 20, more usually about 8 to about 50 in which a sequence under consideration is compared to a reference sequence of the same number of contiguous positions after the two sequences are optimally aligned. The comparison window may comprise additions or deletions (i.e., gaps) of about 20% or less as compared to the sequence under consideration (which does not comprise additions or deletions) or to the reference sequence (which does not comprise additions or deletions) for optimal alignment of the two sequences. Good alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection, or using dot diagrams, and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by an appropriate method. For example, sequence identity analysis may be carried out using the DNASIS computer program (Version 2.5 for windows; available from Hitachi Software engineering Co., Ltd., South San Francisco, Calif., USA) using standard defaults as used in the reference manual accompanying the software.

“Stringency” as used herein refers to the temperature and ionic strength conditions, and presence or absence of certain organic solvents, during hybridization. The higher the stringency, the higher will be the observed degree of complementarity between immobilized polynucleotides and the labeled target polynucleotide.

“Stringent conditions” as used herein refers to temperature and ionic conditions under which only polynucleotides having a high proportion of complementary bases, preferably having exact complementarity, will hybridize. The stringency required is nucleotide sequence dependent and depends upon the various components present during hybridization. Generally, stringent conditions are selected to be about 10 to 20° C. less than the thermal melting point (Tₘ) for the specific sequence at a defined ionic strength and pH. The Tₘ is the temperature (under defined ionic strength and pH) at which 50% of a target sequence hybridizes to a complementary probe. It will be understood that an oligonucleotide probe will hybridize to a target sequence under at least low stringency conditions, preferably under at least medium stringency conditions and more preferably under high stringency conditions. Reference herein to low stringency conditions include and encompass from at least about 1% v/v to at least about 15% v/v formamide and from at least about 1 M to at least about 2 M salt for hybridization at 42 C, and at least about 1 M to at least about 2 M salt for washing at 42 C. Low stringency conditions also may include 1% Bovine Serum Albumin (BSA), 1 mM EDTA, 0.5 M NaHPO₄ (pH 7.2), 7% SDS for hybridization at 65 C, and (i) 2×SSC, 0.1% SDS; or (ii) 0.5% BSA, 1 mM EDTA, 40 mM NaHPO₄ (pH 7.2), 5% SDS for washing at room temperature. Medium stringency conditions include and encompass from at least about 16% v/v to at least about 30% v/v formamide and from at least about 0.5 M to at least about 0.9 M salt for hybridization at 42 C, and at least about 0.5 M to at least about 0.9 M salt for washing at 42 C. Medium stringency conditions also may include 1% Bovine Serum Albumin (BSA), 1 mM EDTA, 0.5 M NaHPO₄ (pH 7.2), 7% SDS for hybridization at 65 C, and (i) 2×SSC, 0.1% SDS; or (ii) 0.5% BSA, 1 mM EDTA, 40 mM NaHPO₄ (pH 7.2), 5% SDS for washing at 42 C. High stringency conditions include and encompass from at least about 31% v/v to at least about 50% v/v formamide and from at least about 0.01 M to at least about 0.15 M salt for hybridization at 42 C, and at least about 0.01 M to at least about 0.15 M salt for washing at 42 C. High stringency conditions also may include 1% BSA, 1 mM EDTA, 0.5 M NaHPO₄ (pH 7.2), 7% SDS for hybridization at 65 C, and (i) 0.2×SSC, 0.1% SDS; or (ii) 0.5% BSA, 1 mM EDTA, 40 mM NaHPO₄ (pH 7.2), 1% SDS for washing at a temperature in excess of 65 C. Other stringent conditions are well known in the art. A skilled addressee will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al. (1989, supra) at sections 1.101 to 1.104.

Reference herein to a “subsequence” refers to a contiguous sequence of a particular unit, value, variable or entity, that exits in part or in whole within a larger contiguous sequence of that particular unit, value, variable or entity. In this context a subsequence can refer to a contiguous sequence of nucleotides or amino acids within, or that is part of, a larger contiguous sequence of nucleotides or amino acids, respectively.

By “substantially complementary” it is meant that an oligonucleotide probe is sufficiently complementary to hybridize with a target sequence. Accordingly, the nucleotide sequence of the oligonucleotide probe need not reflect the exact complementary sequence of the target sequence. In a preferred embodiment, the oligonucleotide probe contains no mismatches and with the target sequence.

The phrase “substantially similar affinities” refers herein to target sequences having similar strengths of detectable hybridization to their complementary or substantially complementary oligonucleotide probes under a chosen set of stringent conditions.

The term “target nucleic acid”, “target nucleic acid sequence”, “target polynucleotide” and the like refer to a polynucleotide of interest (e.g., a single gene or polynucleotide) or a group of polynucleotides (e.g., a family of polynucleotides). The target polynucleotide can designate mRNA, RNA, cRNA, cDNA or DNA. The probe is used to obtain information about the target polynucleotide: whether the target polynucleotide has affinity for a given probe. Target polynucleotides may be naturally occurring or man-made nucleic acid molecules. Also, they can be employed in their unaltered state or as aggregates with other species. Target polynucleotides may be associated covalently or non-covalently, to a binding member, either directly or via a specific binding substance. A target polynucleotide can hybridize to a probe whose sequence is at least partially complementary to a subsequence of the target polynucleotide.

The term “target oligonucleotide sequence” is used herein to refer to a chosen nucleotide sequence of at most 300, 250, 200, 150, 100, 75, 50, 30, 25 or at most 15 nucleotides in length. Target oligonucleotide sequences include sequences of at least 8, 10, 15, 25, 30, 35, 45, 50, 60, 70, 80, 90, 100, 120, 135, 150, 175, 200, 250 and 300 nucleotides in length.

As used herein the term “tiling path” refers to a path for reconstructing a target subunit sequence from a set of overlapping subsequences, of length p, by tiling a first subsequence to produce a tiled first sequence and selecting a second subsequence from the set which overlaps with the tiled first sequence by q subunits and which comprises additional sequence of p-q to the left or to the right of said tiled first sequence and tiling the additional sequence to the right or to the left of the tiled first sequence to form a tiled second sequence and iteratively continuing from the tiled second sequence.

2. A New Paradigm for Experimental Data Collection, Processing and Analysis

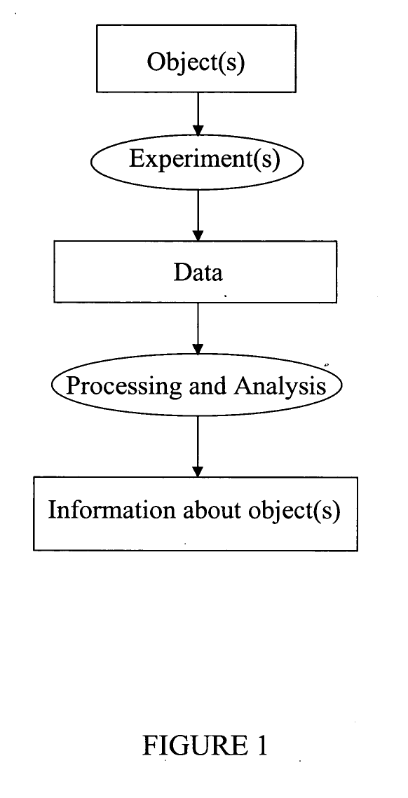

The present invention provides a new paradigm, designated SAM (Sequence Analysis via Mutagenesis), for experimental data collection, processing and analysis and has many applications. Most experimental data collection, processing and analysis can be described by the flow chart shown in FIG. 1. In this chart, rectangles represent objects such as molecules or data sets, and ovals represent processes. The rectangle at the top represents the object or objects about which one aims to obtain information. For example, in DNA sequencing the object is a DNA molecule and the desired information is the primary sequence of the molecule. One or more experiments are performed to obtain data about the object(s) and this data is then processed and analyzed to obtain the required information, possibly with some errors.

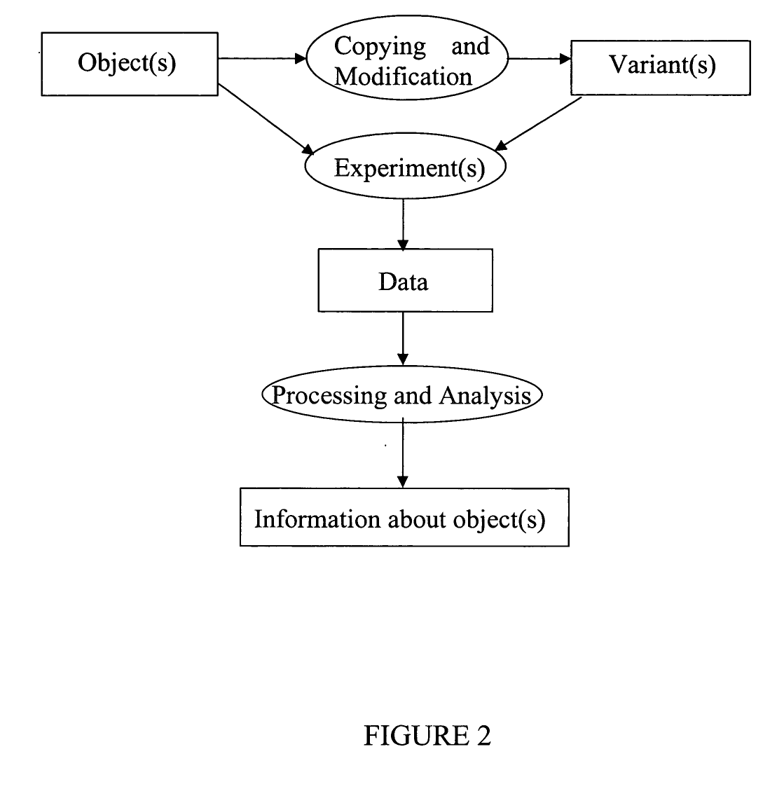

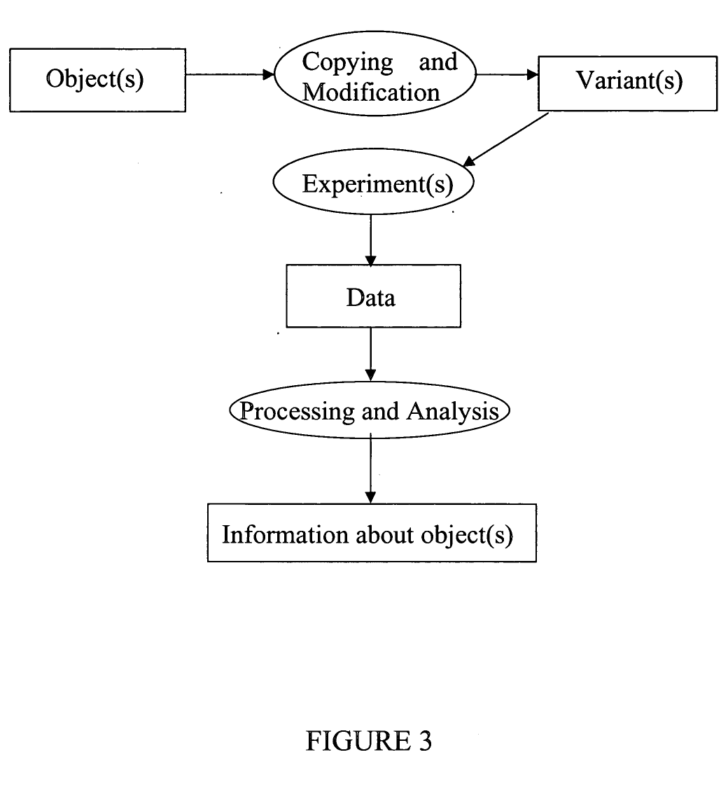

In accordance with the present invention a new paradigm is provided, which is illustrated in FIG. 2. The new feature in this paradigm is the generation of modified copies (or variants) of the original objects. The original object(s) and the variant(s) are then subjected to various experimental procedures. Alternatively, experiments may be performed only on the variants as in FIG. 3. The resulting data is then analyzed or processed to infer or otherwise obtain information about the original object(s), embodiments of which will be described hereinafter.

One reason for generating variants is that they may be amenable to experimentation in ways that the original object(s) were not. Thus one may obtain data from the variants that would be difficult or impossible to obtain from the original object(s). However, the fact that the data pertains to a variant, rather than to the original object(s), must be taken into account and corrected for during the processing and analysis of the data.

The above strategy has many applications. For example, the strategy may be applied to the analysis of subunit sequences to infer or otherwise obtain information relating to a property or feature or physical parameter of the subunit sequence, including but not restricted to, its sequence information, structure, size or refractory behavior to the execution of a task thereon (e.g., cloning or sequencing). In one embodiment, the strategy is applied advantageously to analyzing the refractory behavior of a primary subunit sequence to the execution of a task. In this embodiment the behavior of one or more secondary subunit sequences which vary from the primary subunit sequence by the addition, deletion and/or substitution of at least one subunit is analyzed by determining whether the variation in the secondary subunit sequence(s) renders the task wholly or partially executable on the secondary subunit sequence. Accordingly, the method permits an analysis of particular characteristics in the primary subunit sequence, which render it refractory to the execution of a task. For example, a task to which a nucleic acid sequence may be refractory includes, but is not restricted to, cloning, amplification and sequencing. In this regard, it is well-known that some DNA regions interfere with vector or host functions (Bieth et al, 1997; Williamson et al, 1993), which renders them refractory to cloning. Other regions are known to throw off sequencing enzymes presumably due to an unusually high or low GC content (Perng et al, 1994). Still other regions contain a number of direct or inverted repeats (The Sanger Centre 1998; Chissoe et al, 1997). Direct repeats cause problems for PCR because priming sites must lie in single copy sequence. Inverted repeats can cause problems because they lead to base pairing between different regions of a single stranded DNA molecule. The presence of inverted repeats has been identified as a significant cause of poor sub-clone coverage (Chissoe et al, 1997). In general, “unclonable” and “unsequenceable” regions are often repeat sequence elements, which may also be GC- or AT-rich, or contain kinkable DNA sites (Razin et al, 2001 Journal of Molecular Biology 307:481-486). For example, small known unclonable DNA fragments, such as the unstable regions found at human 22q11 and 11q23 (Kurahashi et al, 2000 Human Molecular Genetics 9:1665-1670), the human immunoglobulin heavy chain gene cluster (Kang and Cox, 1996 Genomics 35:189-195), the human growth hormone gene (Bieth et al, 1997 Gene 194:97-105), intergenic spacer located between the pi and alpha(D) chicken alpha-type globin genes (Razin et al, 2001). Journal of Molecular Biology 307:481-486), and a 22 kb element from the yeast genome (Voet et al, 1997 Yeast 13:177-182) conform to these patterns.

When a primary nucleic acid sequence is refractory, for instance, to any or all of the above tasks, it is possible to analyze its sequence characteristics underlying the refractory behavior by analyzing variant nucleic acid sequences according to the invention, on which the task (e.g., sequencing or cloning) is wholly or partially executable. For example, inverted repeats or palindromes, which may be present in the primary nucleic acid sequence, may be modified in the variant nucleic acid sequences such that formation of stem-and-loop structures is prevented, reduced or otherwise weakened. Sequencing of several sequence variants and subsequent alignment of the sequences can permit the deduction of a consensus sequence, which corresponds to a whole or partial sequence of the primary nucleic acid sequence. The sequence information so obtained may also provide the means to identify local sequence characteristics (e.g., palindromic sequences) underlying the poor clonability of the primary subunit sequence and to, thereby, facilitate the cloning of the primary nucleic acid sequence in parts.

3. Sequence Analysis Using SAM

3.1. General Overview

The invention features a method for wholly or partially deducing the sequence of a target subunit sequence. The method broadly comprises comparing the individual sequences of a plurality of variants, which are distinguished individually from the target subunit sequence by the addition, deletion and/or substitution of at least one subunit, with each other and optionally with a sequence derived from the target subunit sequence or from a sequence adjacent thereto to deduce information about the target subunit sequence, which corresponds to all or part of the target subunit sequence. The comparison may be effected using any suitable technique that compares sequence information. Such techniques include, but are not restricted to, sequence alignment and probabilistic techniques as for example described herein.

This method may be applied to a variety of sequencing techniques including, but not limited to, shotgun sequence analysis and SBH. In a preferred embodiment of this type, the method comprises alternately reconstructing the target subunit sequence and the variant subunit sequence(s) using an end portion of a respective reconstruction as a guide to extend another reconstruction. For example, a portion of the primary subunit sequence may be compared with subsequences corresponding to the variant subunit sequence(s) to identify a subsequence which aligns best with that portion and which extends unambiguously in said alignment a reconstruction of one or more variant subunit sequences beyond the said portion to form a reconstruction of the variant subunit sequence(s). An end portion of that reconstruction is then compared with subsequences corresponding to the target subunit sequence to identify a subsequence which aligns best with the end portion of that reconstruction and which extends unambiguously in said alignment the reconstruction of said primary subunit sequence. Suitably, the method comprises deducing a best alignment between a subsequence and a sequence reconstruction by comparing the alignment of different subsequences with said reconstruction to produce a plurality of extended reconstructions together with individual alignment scores for each reconstruction, and optionally iteratively comparing downstream alignments of extended reconstructions using subsequences available for reconstruction, and determining a reconstruction with the highest scoring alignment to thereby deduce said best alignment.

In another embodiment, this method can be used advantageously to deduce the sequence of at least a portion of a target subunit sequence that is refractory to sequence analysis. The method broadly involves providing a plurality of variants whose individual sequences are distinguished from the target subunit sequence by the addition, deletion and/or substitution of at least one subunit, wherein the variation is associated with the abrogation, inhibition or otherwise amelioration of said refractory behavior. The variants are then sequenced, in whole or in part, to provide a sequence for each variant, and the individual sequences of the variants are then compared with each other and optionally with a sequence flanking the target subunit sequence to deduce a consensus sequence, which corresponds to all or part of the target subunit sequence.

In another embodiment, the invention features a method for unambiguously extending an incomplete reconstruction of a primary subunit sequence by comparing overlapping subsequences corresponding to said primary subunit sequence, wherein said reconstruction is incompletable due to the presence of repeated subsequences in the primary subunit sequence. The method comprises the steps of: (a) providing at least one secondary subunit sequence which varies from said primary sequence by the addition, deletion and/or substitution of at least one subunit, wherein said variation is associated with the alteration or destruction of at least one of said repeated subsequences, and (b) comparing overlapping subsequences corresponding to said at least one secondary subunit sequence and to said primary subunit sequence, to unambiguously extend said incomplete reconstruction. In a preferred embodiment, some or all of the subsequences of said at least one secondary subunit sequence, which are varied relative to the repeated subsequences, are different relative to each other. In another preferred embodiment, the method comprises comparing an end portion of said incomplete reconstruction with one or more subsequences corresponding to said at least one secondary subunit sequence to deduce an unambiguous extension to said incomplete reconstruction. In yet another embodiment, the method comprises alternately reconstructing said primary subunit sequence and said at least one secondary subunit sequence using an end portion of a respective reconstruction as a guide to extend another reconstruction. In a preferred embodiment of this type, the method comprises first comparing an end portion of the incomplete reconstruction with subsequences corresponding to said at least one secondary subunit sequence to identify a subsequence which aligns best with said end portion and which extends unambiguously in said alignment a reconstruction of said at least one secondary subunit sequence beyond the incomplete reconstruction of said primary subunit sequence to form an extended reconstruction of said at least one secondary subunit sequence. An end portion of the extended reconstruction is then compared with subsequences corresponding to said primary subunit sequence to identify a subsequence which aligns best with said end portion of said extended reconstruction and which extends unambiguously in said alignment the incomplete reconstruction of said primary subunit sequence to form an extended reconstruction of said primary subunit sequence.

In a preferred embodiment, the subunit sequence is selected from a nucleic acid sequence or from an amino acid sequence. In an especially preferred embodiment of this type, the subunit sequence is a nucleic acid sequence.

When the subunit sequence is a nucleic acid sequence, it will be appreciated by those of skill in the art that useful sequence information need not necessarily be obtained from a variant or secondary nucleic acid sequence whose sequence corresponds to the target or primary nucleic acid sequence. In this regard, it will be recognized that a variant or secondary nucleic acid sequence may correspond to a complementary sequence of the target or primary nucleic acid sequence and may, therefore, be distinguished from that complementary sequence by the addition, deletion and/or substitution of at least one nucleotide. Sequence analysis of the secondary nucleic acid sequence will provide sequence information which can be used to deduce the complementary sequence of the variant or secondary nucleic acid sequence, which complementary sequence could be used solve, or extend the reconstruction of, the target or primary nucleic acid sequence.

The variant or secondary subunit sequences may already exist and could, therefore, constitute naturally occurring variants (e.g., different alleles of a gene, different polymorphic forms of a polymorphic site, homologous or orthologous genes in different organisms). Alternatively, the variant or secondary subunit sequences may be produced by mutagenesis techniques as for example described infra.

The inventors have designated the above method of sequence analysis Sequence Analysis via Mutagenesis (SAM) and reference to mutants, mutations, mutated and the like will be understood to include any variant, or sequence variation, relative to a reference subunit sequence, which is naturally-occurring or pre-existing or the result of mutagenesis techniques. In one embodiment of SAM, a general method is provided that can be used to improve the speed, accuracy and effectiveness of a wide variety of nucleic acid sequencing technologies. The SAM method is applicable in the following situation. For example, let G be a genome or part of a genome and let seq(G) be the unknown sequence of G. Suppose that G has been partially sequenced and that consequently there is a set Ω_{G} of known subsequences, each of which is homologous to a subsequence of seq(G) but possibly contains some errors. The position of some of these subsequences relative to seq(G), or relative to other subsequences in Ω_{G}, may be known or approximated. However, seq(G) cannot be completely reconstructed because there are gaps (parts of seq(G) not covered by any of the subsequences) or ambiguities (alternative arrangements of the subsequences) or both. Further information is required to complete the sequence, but obtaining that information is hampered in some way by the presence of repeated motifs and other problematic regions of sequence. (Note that Ω_{G} may be empty, and that the method can, therefore, be applied even from the very beginning of a sequencing project.)

Generally, the foregoing is a description of any incomplete sequencing project, regardless of the sequencing technology employed in which the presence of problem regions hampers the further advancement of the project. In practice, most sequencing technologies do not perform well on certain types of sequence, so the aforesaid description is widely applicable.

The essential concept of SAM is to partially sequence a number of mutants of G and use the extra information thus obtained to assist in reconstructing seq(G). The mutants could be naturally-occurring in different lineages, or manufactured in the laboratory. To explain this concept detail, it will be helpful to introduce the following notation. Let n be the number of mutants and let M₁, M₂, . . . , Mₙ be the mutants. Let seq(Mᵢ) be the unknown sequence of Mᵢ. Let Ωᵢ be a set of strings obtained by partially sequencing Mᵢ. Like the strings in Ω_{G}, the strings in Ωᵢ may contain some errors. It should be noted, however, that Ωᵢ also includes within its scope only one subsequence, namely the sequence of mutant i.

There are two reasons for providing or generating the mutants. The first reason is that the mutants may contain fewer problem regions than the original or target sequence and should, therefore, be easier to sequence. For example, genomic DNA is highly repetitive, so random mutation is more likely to destroy repeats than to create them. The second reason is that each mutant contains a different pattern of problem regions. It is possible that some regions of Mᵢ will be more difficult to sequence because new repeats or other problem regions appear in the mutant. However, the important thing is that different regions of M₁, M₂, . . . , Mₙ and G are easy to sequence.

To take advantage of the additional information provided by sequencing the mutants, the present method relies in one embodiment on forming highly probable alignments between strings or subsequences in Ω₁, Ω₂, . . . , and Ω_{G}. These alignments fix the positions of some subsequences relative to seq(G) and to other subsequences. For example, in the diagram below, two subsequences from Ω_{G} have been aligned to a subsequence from Ω₁. Although there is an overlap of four bases between the subsequences from Ω_{G}, they could not be joined because the overlap occurs within a motif CCGTTG that is repeated elsewhere in seq(G) and consequently there are alternative positions for each subsequence. The alignment to a subsequence from Ω₁ resolves any ambiguities and makes it clear that these two subsequences overlap. Moreover, if the position of any one of these three subsequences relative to seq(G) is known, then the positions of the other two subsequences relative to seq(G) may be inferred.

| | | |
|---|---|---|
| subsequences from Ω_{G}: | CGCTTCATGAATGGTCCGTT | [SEQ ID NO:1] |
| | GGTTGCTTATTCAAGTGC | [SEQ ID NO:2] |
| subsequence from Ω₁: | CAAGAATATTCGGTTGATTACTCA | [SEQ ID NO:3] |

The method can also be used to close gaps in the reconstruction of seq(G). For example, the diagram below shows subsequences from three mutants aligned to the end of a string in Ω_{G}, adjacent to a gap. By taking a consensus of the strings from Ω₁, Ω₂, and Ω3 the reconstruction can be extended into the gap. It is highly probable that the gap shown here begins with the subsequence GTAGTGA [SEQ ID NO:4].

| | | |
|---|---|---|
| subsequence from Ω_{G}: | CAGCATCCGCTGTGCCTGGAGCACA ...... (Gap) ...... | [SEQ ID NO:5] |
| subsequence from Ω₁: | GAACCACAGTAGTGAAATGAC | [SEQ ID NO:6] |
| subsequence from Ω₂: | AGCCGGGGCCACAGGAGTGA | [SEQ ID NO:7] |
| subsequence from Ω₃: | GTATGCCTGGAACCCTGTAGTGATCAG | [SEQ ID NO:8] |

Note that it is possible to align subsequences from Ω₁, Ω₂, . . . , and Ωₙ and take a consensus without even using subsequences from ΩG. The method can, therefore, be applied even if Ω_{G} is not available. In some cases, the original sequence may be so full of repeats that it is desirable to dispense with G and apply the method using only the mutants.

In summary, the sub-tasks of the method are as follows. Note that they do not necessarily have to be carried out consecutively; several of the steps may overlap.

1. Obtain n mutants M₁, M₂, . . . , Mₙ of G.

2. Partially sequence G and partially or wholly sequencing the mutants to obtain sets of subsequences Ω₁, Ω₂, . . . , Ωₙ and Ω_{G}.

3. Identify probable alignments amongst the strings or subsequences in Ω₁, Ω₂, . . . , Ωₙ and Ω_{G}.

4. Use the alignments to position subsequences relative to seq(G) and to each other.

5. Where there is a gap in the reconstruction of seq(G), optionally obtain a consensus of any mutated subsequences that cover all or part of the gap.

6. Optionally analyze base statistics at each site to determine the quality of the consensus, and to identify possible sites at which there are sequencing errors or polymorphisms.

3.2. Sequencing by Hybridization (SBH)

SBH typically involves (a) interrogation of an unknown target nucleic acid sequence with a set of oligonucleotide probes, (b) detection of probes that hybridize, and hence are complementary, to subsequences of the target sequence. This determines the subsequence content or SBH spectrum of the target sequence, and reconstruction of the target sequence from its SBH spectrum by use of an appropriate combinatorial algorithm. This process is called SBH reconstruction.

SBH, in its standard format, uses a complete set of 4^{p} probes of length p. The usual approach is to synthesize the probes in a fixed pattern or array and to fluorescently tag copies of the target DNA fragment, so that those probe sites to which it binds are identified. Repeated motifs are the major obstacle to effective SBH because they induce reconstruction ambiguities. A recent unpublished study conducted by the inventors showed that repeated motifs in human genetic sequences severely limit the effectiveness of SBH. In general, when probes of length p are used, ambiguities arise if the target fragment contains repeated motifs of length p−1. The following example illustrates this in more detail. Consider the following sequence: CCTGAGATCGCTTCGTGAATGGTCCGTTGCTTATTCAAGTGCTTTACCAC [SEQ ID NO:9]

Using probes of length p=5, the SBH spectrum of this sequence consists of the following subsequences.

| | | |
|---|---|---|
| CCTGA [SEQ ID NO:10] | CGCTT [SEQ ID NO:11] | GAATG [SEQ ID NO:12] |
| CGTTG [SEQ ID NO:13] | ATTCA [SEQ ID NO:14] | CTTTA [SEQ ID NO:15] |
| CTGAG [SEQ ID NO:16] | GCTTC [SEQ ID NO:17] | AATGG [SEQ ID NO:18] |
| GTTGC [SEQ ID NO:19] | TTCAA [SEQ ID NO:20] | TTTAC [SEQ ID NO:21] |
| TGAGA [SEQ ID NO:22] | CTTCG [SEQ ID NO:23] | ATGGT [SEQ ID NO:24] |
| TTGCT [SEQ ID NO:25] | TCAAG [SEQ ID NO:26] | TTACC [SEQ ID NO:27] |
| GAGAT [SEQ ID NO:28] | TTCGT [SEQ ID NO:29] | TGGTC [SEQ ID NO:30] |
| TGCTT [SEQ ID NO:31] | CAAGT [SEQ ID NO:32] | TACCA [SEQ ID NO:33] |
| AGATC [SEQ ID NO:34] | TCGTG [SEQ ID NO:35] | GGTCC [SEQ ID NO:36] |
| GCTTA [SEQ ID NO:37] | AAGTG [SEQ ID NO:38] | ACCAC [SEQ ID NO:39] |
| GATCG [SEQ ID NO:40] | CGTGA [SEQ ID NO:41] | GTCCG [SEQ ID NO:42] |
| CTTAT [SEQ ID NO:43] | AGTGC [SEQ ID NO:44] | ATCGC [SEQ ID NO:45] |
| GTGAA [SEQ ID NO:46] | TCCGT [SEQ ID NO:47] | TTATT [SEQ ID NO:48] |
| GTGCT [SEQ ID NO:49] | TCGCT [SEQ ID NO:50] | TGAAT [SEQ ID NO:51] |
| CCGTT [SEQ ID NO:52] | TATTC [SEQ ID NO:53] | GCTTT [SEQ ID NO:54] |

Starting with the probe CCTGA [SEQ ID NO:55] and adding probes one at a time to the right-hand end, one can reconstruct the subsequence CCTGAGATCGCTT [SEQ ID NO:56]. At this point an ambiguity arises because there are three possible probes that could be added next: GCTTC [SEQ ID NO:57], GCTTA [SEQ ID NO:58] or GCTTT [SEQ ID NO:59]. The ambiguity arises because of the presence of the repeated 4-mer GCTT [SEQ ID NO:60].

Other subsequences can be reconstructed by starting with a different probe and adding probes at both ends until either no more probes can be added or an ambiguity arises. In this manner, the following subsequences can be obtained.

| | |
|---|---|
| CCTGAGATCGCTT | [SEQ ID NO:61] |
| GCTTATTCAAGTGCTT | [SEQ ID NO:62] |
| GCTTCGTGAATGGTCCGTTGCTT | [SEQ ID NO:63] |
| GCTTTACCAC | [SEQ ID NO:64] |

These subsequences cover the target sequence without gaps. However, the arrangement of the subsequences and the number of times each one is repeated cannot be inferred without additional information. SAM, however, can be used to obtain the required information. Consider the mutants shown below. Twelve of the fifty bases have been substituted—an error level of 0.24. CCAAAGATCGCTTCAAGAATATTCGGTTGATTACTCAAGAGCCTCACCAC [SEQ ID NO:65]

Using probes of length five and performing subsequence reconstructions in a similar manner to that described above, the following subsequences are obtained.

| | |
|---|---|
| CCAAAGA | [SEQ ID NO:66] |
| AAGAATATTCGGTTGATTACTCA | [SEQ ID NO:67] |
| AAGAGCCTCA | [SEQ ID NO:68] |
| AAGATCGCTTCAAGA | [SEQ ID NO:69] |
| CTCAAGA | [SEQ ID NO:70] |
| CTGACCAC | [SEQ ID NO:71] |

In what follows, the original sequence is referred to as sequence A and its growing reconstruction as reconstruction A. The mutant is referred to as sequence B and its growing reconstruction as reconstruction B. The first subsequence in sequence A must be CCTGAGATCGCTT [SEQ ID NO:72], since none of the other subsequences can overlap the beginning of it by four characters or more. Similarly, the first subsequence in sequence B must be CCAAAGA [SEQ ID NO:73]. These subsequences can be aligned as follows:

A) CCTGAGATCGCTT [SEQ HDNO:74]

B) CCAAAGA [SEQ ID NO:75]

The strategy in this example will be to extend the shorter reconstruction with the subsequence that aligns best to the longer reconstruction. Reconstruction B is currently shorter; there are three possible ways to extend it, using one of the subsequences AAGAATATTCGGTTGATTACTCA [SEQ ID NO:76], AAGAGCCTCA [SEQ ID NO:77], or AAGATCGCTTCAAGA [SEQ ID NO:78]. (These are the only options because it can be shown that adjacent subsequences must overlap by at least four characters.) To decide which subsequences to extend with, consider how well each one aligns with the six bases at the end of reconstruction A. The third subsequence matches all six bases and is clearly the most probable extension. The reconstructions can now be aligned like this:

| | |
|---|---|
| A) CCTGAGATCGCTT | [SEQ ID NO:79] |
| B) CCAAAGATCGCTTCAAGA | [SEQ ID NO:80] |

Now A is the shorter reconstruction. It can be extended in one of three ways, using one of the subsequences GCTTATTCAAGTGCTT [SEQ ID NO:81], GCTTCGTGAATGGTCCGTTGCTT [SEQ ID NO:82] or GCTTTACCAC [SEQ ID NO:83]. The second subsequence aligns best with the five bases at the end of reconstruction B. The alignment is now:

| | |
|---|---|
| A) CCTGAGATCGGTTCGTGAATGGTCCGTTGCTT | [SEQ ID NO:84] |
| B) CCAAAGATCGCTTCAAGA | [SEQ ID NO:85] |

Continuing the same style of reasoning, the next four extensions are AAGAATATTCGGTTGATTACTCA [SEQ ID NO:86] on reconstruction B, GCTTATTCAAGTGCTT [SEQ ID NO:87] on reconstruction A, CTCAAGA [SEQ ID NO:88] on reconstruction B, and AAGAGCCTCA [SEQ ID NO:89] on reconstruction B. The reconstructions can now be aligned as follows:

| | |
|---|---|
| A) CCTGAGATCGCTTCGTGAATGGTCCGTTGCTTATTCAAGTGCTT | [SEQ ID NO:90] |
| B) CCAAAGATCGCTTCAAGAATATTCGGTTGATTACTCAAGAGCCTGA | [SEQ ID NO:91] |

The next extension is more difficult. There are only two bases of reconstruction B overhanging the end of the alignment. Two extensions of reconstruction A match this overhang equally well, namely GCTTCGTGAATGGTCCGTTGCTT [SEQ ID NO:92] and GCTTTACCAC [SEQ ID NO:93]. However, a decision can still be made by simultaneously considering the possible extensions of reconstruction B, namely CTCAAGA [SEQ ID NO:94] and CTCACCAC [SEQ ID NO:95]. There are four possible ways to pair an extension of A to an extension of B; the best alignment is achieved using GCTTTACCAC [SEQ ID NO:96] for reconstruction A and CTCACCAC [SEQ ID NO:97] for reconstruction B. It might sometimes be necessary to look ahead several extensions to determine the best alignment.

Neither reconstruction can be extended further, so the final alignment, in which both the original sequence and its mutant have been correctly reconstructed, is:

| | |
|---|---|
| A) CCTGAGATCGCTTCGTGAATGGTCCGTTGCTTATTCAAGTGCTTTACCAC | [SEQ ID NO:98] |
| B) CCAAAGATCGCTTCAAGAATATTCGGTTGATTACTCAAGAGCCTCACCAC | [SEQ ID NO:99] |

The reconstruction was possible only because the sequences contain different patterns of repeated (p−1)-mers. Two occurrences of GCTT [SEQ ID NO:100] were destroyed in the mutant: one was changed to GATT [SEQ ID NO:101], the other to GCCT [SEQ ID NO:102]. However, the mutant contains the repeated 4-mers AAGA [SEQ ID NO:103] and CTCA [SEQ ID NO:104]. Both sequences therefore contain reconstruction ambiguities, but in different places. Consequently, there were overhanging bases at each stage of the reconstruction, and these could be used to select the next subsequence in the manner described.

This example was fortunate in that the 4-mer GCTT [SEQ ID NO:105], which was repeated in the original sequence, was not repeated in the mutant. In general, some repeats may survive mutation and consequently a single mutant might not resolve all reconstruction ambiguities. Multiple mutants should, therefore, be generated.

Provided that at least one mutant does not contain any repeats in a given region, there will be a maximal subsequence of that mutant spanning that region. This subsequence can, therefore, be used to help resolve reconstruction ambiguities in that region. In a preferred embodiment, a parent nucleic acid sequence is mutagenized to produce at least one variant nucleic acid sequence in which at least 5%, preferably at least 10%, more preferably at least 20%, even more preferably at least 30%, and still even more preferably at least 40% of nucleotides are different relative to the parent nucleic acid sequence.

The final extension of the above example demonstrated that aligning subsequences to the overhang of the longer reconstruction does not always resolve the ambiguity successfully. Two or more subsequences may align equally well to the overhanging bases, and even if one subsequence aligns better than the others do, it may not be the correct extension. The reconstruction algorithm should therefore ‘look ahead’ to see how well later extensions align. Ideally, the reconstruction algorithm should compare possible full-length reconstructions of the original sequence to possible full-length reconstructions of the mutant(s). Thus, alignments between pairs of subsequences may not resolve ambiguities in as convincing a manner as alignments amongst groups of subsequences.

Thus, from the foregoing, the invention broadly contemplates a method for unambiguously extending an incomplete reconstruction of a subunit sequence by comparing overlapping subsequences, of length p, corresponding to said primary subunit sequence, wherein said reconstruction is incompletable due to the presence of repeated subsequences of length p−1 in said primary subunit sequence. The method comprises providing at least one secondary subunit sequence which varies from said primary subunit sequence by the addition, deletion and/or substitution of at least one subunit, wherein said variation is associated with the alteration or destruction of at least one of said repeated subsequences, and comparing overlapping subsequences, of length p, corresponding to said at least one secondary subunit sequence and to said primary subunit sequence, to unambiguously extend said incomplete reconstruction.

Suitably, the method comprises comparing an end portion of said incomplete reconstruction with one or more subsequences corresponding to said at least one secondary subunit sequence to deduce an unambiguous extension to said incomplete reconstruction.

The method preferably comprises alternately reconstructing said primary subunit sequence and said at least one secondary subunit sequence using an end portion of a respective reconstruction as a template or guide to extend another reconstruction.

In a preferred embodiment of this type, the method comprises comparing an end portion of said incomplete reconstruction with subsequences corresponding to said at least one secondary subunit sequence to identify a subsequence which aligns best with said end portion and which extends unambiguously in said alignment a reconstruction of said at least one secondary subunit sequence beyond the incomplete reconstruction of said primary subunit sequence to form an extended reconstruction of said at least one secondary subunit sequence, and comparing an end portion of said extended reconstruction with subsequences corresponding to said primary subunit sequence to identify a subsequence which aligns best with said end portion of said extended reconstruction and which extends unambiguously in said alignment the incomplete reconstruction of said primary subunit sequence to form an extended reconstruction of said primary subunit sequence.

In another embodiment, the method preferably comprises deducing a best alignment between a subsequence and an incomplete reconstruction by comparing alignment of different subsequences with said incomplete reconstruction to produce a plurality of extended reconstructions together with individual alignment scores for each reconstruction, and optionally iteratively comparing downstream alignments of extended reconstructions using subsequences available for reconstruction, and determining a reconstruction with the highest scoring alignment to thereby deduce said best alignment.

Alignment algorithms are well known in the art. For example, reference may be made to sequence alignment and assembly algorithms and software including, but not restricted to, PHRAP (Green, 1996), TIGR Assembler (Sutton et al, 1995), CAP (Huang and Madan, 1999), FAK (Myers et al, 1996), and STROLL (Chen and Skiena, 2000), SBH alignment and assembly algorithms (Pevzner, 1989 and 1995, Preparata et al, 1999) and (Pe'er and Shamir 2000).

Generally, an alignment of two subsequences A and B is obtained by first inserting spaces either into or at the ends of A and B, and then placing the two resulting subsequences one above the other so that every character or space in either subsequence is opposite a unique character or a unique space in the other subsequence. A scoring scheme for alignments is a method for associating a unique value (usually an integer, but sometimes a real number) with every alignment. One way to score an alignment is to count the number of mismatches and spaces in the alignment. With this scoring scheme, it is actually low-scoring alignments that are highly similar. String similarity is a more general approach to scoring alignments that relies on the following definitions:

1. Let X be the alphabet used for strings A and B, and let X′ be X with the addition of an added character denoting a space. Then for any two characters x,y in X′, s(x,y) denotes the score obtained by aligning character x against character y.

2. For a given alignment of A and B, let A′ and B′ denote the strings or sequences after the chosen insertion of spaces. The score of the alignment is defined as the sum of s(A′(i),B′(i)) over all positions i, where A′(i) and B′(i) are the characters at the i^{th} positions of A′ and B′, respectively.

Usually, but not always, the score s(x,y) is greater than or equal to zero if x and y match, and negative if x and y mismatch. In that case, it is high-scoring alignments that indicate strong similarity.

A still more general scoring scheme includes a penalty term for gaps. A gap is any maximal, consecutive run of spaces in a single string of a given alignment. Such scoring schemes first compute the sum of s(A′(i),B′(i)) over all positions i, then subtract an amount f(q) for each gap in the alignment, where q is the length of said gap. The function f(q) is most often a constant Wg or a linear function Wg+q*Ws, but there are many other possibilities in use, such as Wg+log(q).

Thus, a high-scoring alignment of two strings A and B, for a given scoring scheme, is an alignment whose score is high relative to the scores of all other alignments of the strings A and B computed using the same scoring scheme. In practice, an alignment is considered high if it exceeds some threshold value, the value of which will depend on the application.

The above alignment methods are typically used for global alignments of two strings A and B. In many applications of SAM, determination of local alignments will be important. A local alignment of two strings A and B is a global alignment of a sub-string or subsequence of A with a sub-string or subsequence of B. Local alignments can be scored in the same way as global alignments.

It will be understood, however, that the present invention is not dependent on any particular alignment method, algorithm or scoring scheme.

The invention also envisions a method of forming an extension to an incomplete tiling path of overlapping subsequences, of length p, corresponding to a primary target subunit sequence comprising repeated subsequences of length p−1: The method comprises providing at least one secondary subunit sequence which varies from said primary sequence by the addition, deletion and/or substitution of at least one subunit, wherein said variation is associated with the alteration or destruction of at least one of said repeated subsequences, and comparing overlapping subsequences, of length p, corresponding to said at least one secondary subunit sequence and to said primary subunit sequence to extend said incomplete tiling path.

In a preferred embodiment, the subunit sequence is selected from a nucleic acid sequence or an amino acid sequence.

3.3. Shotgun Sequencing

Shotgun sequencing is a nucleic acid sequencing technique in which a long target sequence is pieced together from a collection of short fragments. It typically involves (a) shearing of the target into small fragments, (b) size-selection and cloning of short DNA fragments, typically 0.4-1.2 kb, (c) sequencing the fragments, and (d) identifying overlapping subsequences and joining them to construct long contiguous sequences called ‘contigs’.

Additional features, notably the pairwise end strategy (Weber and Myers, 1997), may be incorporated to render reconstruction more efficient, but the above steps are generally essential to the method. To reduce the number of gaps between contigs and to facilitate the reconstruction phase, the sum of the lengths of the fragments is generally several times the length of the target, so that multiple coverage is obtained. Shotgun sequencing is the most common method for sequencing long DNA clones, which range in size from around 30 kb (cosmid clones) up to around 150 kb (BAC clones). It has also been used to sequence entire genomes, and is the basis of a commercial approach to sequencing the human genome (Venter et al., 2000). Recent analyzes of whole-genome shotgun sequencing are provided by (Weber and Myers, 1997; Green, 1997; Siegel et al., 2000).

Four main problems are encountered in the reconstruction phase: gaps, repeats, polymorphisms and data anomalies (Green, 1997). The potential uses of SAM to close gaps and to detect data anomalies and polymorphisms are discussed infra. Described below is an example of how SAM can be used to solve reconstruction problems caused by repeats. When the sequence contains repeats, it may be possible to assemble the fragments in more than one way. For example, consider the following sequence. TTGAGATTCCTATATATGTTCGATTCCTATATATGAGGATTCCTATATAA [SEQ ID NO:106]

Note that this sequence contains three copies of the motif GATTCCTATATA [SEQ ID NO:107] (underlined). Now suppose that the following fragments have been sequenced.

| | |
|---|---|
| TTGAGATTCCTA | [SEQ ID NO:108] |
| TTCCTATATATGTT | [SEQ ID NO:109] |
| ATATGTTCGATTCCTA | [SEQ ID NO:110] |
| TTCCTATATATGAG | [SEQ ID NO:111] |
| ATATGAGGATTCC | [SEQ ID NO:112] |
| ATTCCTATATAA | [SEQ ID NO:113] |

There are highly probable overlaps between the second and third fragments and between the fourth and fifth fragments. The problem can therefore be reduced to assembling the following subsequences:

| | |
|---|---|
| TTGAGATTCCTA | [SEQ ID NO:114] |
| TTCCTATATATGTTCGATTCCT | [SEQ ID NO:115] |
| ATTCCTATATATGAGGATTCC | [SEQ ID NO:116] |
| ATTCCTATATAA | [SEQ ID NO:117] |

However, there are two plausible ways to assemble these, as shown below.

| | |
|---|---|
| TTGAGATTGCTATATATGTTCGATTCCTATATATGAGGATTCCTATATAA and | [SEQ ID NO:118] |
| TTGAGATTCCTATATATGAGGATTCCTATATATGTTCGATTCCTATATAA. | [SEQ ID NO:119] |

The first of these is the correct assembly.

In realistic shotgun sequencing the target sequence and the fragments would be much longer and the coverage of the sequence would be less uniform (there would be gaps in some places and many overlapping fragments in other places). There would also be errors in the sequences of the fragments. However, the example serves to illustrate the point that there may be more than one way to assemble the fragments when repeated motifs are present. It also demonstrates that this problem occurs when there are no subsequences spanning the repeats.

To illustrate how SAM can be used to resolve the ambiguities, suppose that the following mutant is generated. There are eight substitutions—an error level of 0.16. TTTAGATTCCTATATATATTCGATTTCTGTATATTAGGAATCCCATATTA [SEQ ID NO:120]

Suppose also that the following fragments of the mutant have been sequenced:

| | |
|---|---|
| TTTAGATTCCTAT | [SEQ ID NO:121] |
| TTCCTATATATATTC | [SEQ ID NO:122] |
| TATATTCGATTTCTGTAT | [SEQ ID NO:123] |
| CTGTATATTAGGAAT | [SEQ ID NO:124] |
| TAGGAATCCCATATTA | [SEQ ID NO:125] |

There are convincing overlaps between each pair of adjacent fragments here, so the mutant can be correctly reconstructed without ambiguities. The reconstructed mutant provides a template or guide against which to align the four subsequences from the original sequence. In the diagram below, each of these subsequences is shown optimally aligned to the mutant. (The mutant is the fully reconstructed sequence in the center.) The alignment criterion here is simply to minimize the number of mismatches. Note that each subsequence is correctly aligned and that the original sequence can now be unambiguously reconstructed.

| | |
|---|---|
| TTGAGATTCCTA ATTCCTATATATGAGGATTCC | [SEQ ID NO:126, 127] |
| TTTAGATTGCTATATATATTGGATTTCTGTATATTAGGAATCCCATATTA | [SEQ ID NO:128] |
| TTCCTATATATGTTCGATTGCT ATTCCTATATAA | [SEQ ID NO:129, 130] |

The reconstruction was possible only because the sequences contained different patterns of repeated motifs. Two copies of the repeated motif GATTCCTATATA [SEQ ID NO:131] were modified by mutation, and consequently no subsequences of significant length were repeated in the mutant. This example was fortunate in that the mutant could be fully reconstructed and used as a template. In the more general case, the mutant would only be partially reconstructed. Nevertheless, alignments between subsequences of the original sequence and subsequences of a mutant would resolve some of the ambiguities and allow some subsequences to be joined. If enough mutants are used, it should be possible to resolve all the ambiguities.

The substitution rate used in the above example is possibly higher than would be needed in realistic shotgun sequencing. The repeated motifs that cause problems for shotgun assembly are hundreds or even thousands of nucleotides in length, and are therefore likely to be modified even if the substitution rate is only a few percent.

An important practical detail is the amount of coverage that should be obtained for the target and each of the mutants. By ‘coverage’ is meant the average number of fragments that cover each point of the sequence. It seems likely that a low coverage would be sufficient because each subsequence of a mutant can be regarded as subsequences of the target with a large number of errors. Consequently, covering the target and nine mutants just once each is roughly equivalent to covering the target ten times with an inaccurate sequencing technology.

There is a strong similarity between the applications of the SAM method to shotgun sequencing and to SBH. The short fragments obtained in shotgun sequencing are analogous to the probes obtained in SBH and the contigs assembled in shotgun sequencing are analogous to the maximal subsequences assembled in SBH. The major difference is in scale: contigs are much longer than maximal subsequences and the repeats that cause reconstruction ambiguities for shotgun assembly are much longer than the repeats that hinder SBH reconstruction.

3.4. Gap Closing

A feature common to current genome sequencing technologies is the need to close gaps in an incomplete sequence. Some gaps arise for a statistical reason: the coverage of the sequence is uneven and by pure chance some regions of the sequence have been missed. This type of gap can be closed relatively easily by sequencing additional fragments from the region containing the gap. However, some gaps arise because the genome contains segments of DNA that are refractory to the method of sequencing. This second type of gap is far more difficult to close and requires specialized sequencing strategies (Withgott, 2000).

As mentioned above, there are many reasons why a nucleic acid fragment might be difficult to clone or sequence. It should be possible to solve most of these problems using SAM to modify the problematic sequence. For example, a high GC content can be dealt with by using a mutagen such as bisulphite, which can be used to replace cytosine with thymine.

Direct repeats cause problems for nucleic acid amplification techniques such as the polymerase chain reaction (PCR) because priming sites must lie in single copy sequence, otherwise the PCR will amplify several regions of sequence simultaneously. It can, therefore, be difficult to identify suitable priming sites in a region containing direct repeats. Inverted repeats can cause problems because they lead to base pairing between different regions of a single stranded DNA molecule. These structures can obstruct sequencing in a variety of ways. The presence of inverted repeats has been identified as a significant cause of poor sub-clone coverage (Chissoe et al., 1997).

The following example illustrates the use of SAM to solve a hypothetical sequencing problem caused by an inverted repeat. The first line of the diagram below shows the target sequence and the second line shows two fragments that have been sequenced. The gap between the fragments is difficult to close because it contains the motif CCCGCGCC [SEQ ID NO:132] and its reverse complement GGCGCGGG [SEQ ID NO:133] (both underlined).

| | |
|---|---|
| CAGAAGAAGGAACCCGCGCCCTCTGGGCTGGCGCGGGAGGCTCACACC | [SEQ ID NO:134] |
| CAGAAGAA CTCACACC | [SEQ ID NO:135, 136] |
| CAGATGAAGGAACCCTCGCCGTCTGGGCTGGCGCGGGCGACTCCCACC | [SEQ ID NO:137] |
| AAGAAGAAGAAAACCGCGTCCGCTGGGCTGTTGCGGGAGACTCACAGC | [SEQ ID NO:138] |
| CAAAAGAAGGAACCCGCACCCTCTGTGATGTCGCGGGAGGCTCTCACC | [SEQ ID NO:139] |
| CAGAACAAGGAACCCGCGGCTTCTGGACTGGCGCGAGCGGCTCACATC | [SEQ ID NO:140] |
| CAGAAGAAGGTAGCCGCGCCTTCTGCTCTGGTGCGGGAGGCTCACACG | [SEQ ID NO:141] |

Five mutants of the original sequence are shown, with substitution rates around 0.15. None of the mutants contain the inverted repeat. It is, therefore, assumed that these fragments can be reconstructed in their entirety. By aligning the five sequences as shown and taking a consensus at each site, the original sequence can be correctly reconstructed. Notice, however, that the correct sequence is not obtained by taking a consensus of any four (or fewer) of these mutants. As a general rule, the larger the mutation rate, the more mutations are needed to obtain an error-free consensus (see Section 4.2 for more detail on factors influencing the desired frequency of mutation).

In the above example, the complete sequence of each mutant was available. However, it is sometimes possible to close a gap using only partially sequenced mutants, even when no single fragment spans the gap. The following example illustrates this point.

| | |
|---|---|
| CAGAAGAAGGAACCCGCGCCCTCTGGGCTGGCGCGGGAGGCTCACACC | [SEQ ID NO:142] |
| CAGAAGAA CTCACACC | [SEQ ID NO:143, 144] |
| CAGATGAAGGAACCCTCGCCCTCTGGG | [SEQ ID NO:145] |
| AAACCCCGTCCGCTGGGCTG | [SEQ ID NO:146] |
| AAGAAGGAACCCG GATCTCGCGGGAGGCTCTCACC | [SEQ ID NO:147, 148] |
| CAGAACAAGGAACCCGCG TGGCGCGAGCGGCTCACATC | [SEQ ID NO:149, 150] |
| CCGCGCCTTCTGCTCTGGTGCGGGAGGC | [SEQ IDNO:151] |

Fragments of the five mutants and the original sequence can be aligned convincingly in the manner shown. Where there are gaps in the original, a consensus of the mutant sequences can be taken. This successfully recovers the original sequence.

3.5. Detection of Sequence Errors and Polymorphisms

The primary reason for aligning subsequences in SAM is to infer the positions of subsequences. However, an incidental advantage of the alignments is that they can be used to check for sequencing errors and polymorphisms. For example, a sequence of nine bases is shown aligned to four mutants below. The consensus of the four mutants supports the base-calls at the last eight positions of the original sequence. However, all four mutants have an ‘A’ in the first position, not a ‘T’. This suggests that the ‘T’ in the original sequence could be an error.

| | |
|---|---|
| TGAAGGAAC | [SEQ ID NO:152] |
| AGAAGAAAA | [SEQ ID NO:153] |
| AGAAGGAAC | [SEQ ID NO:154] |
| ACAAGGAAC | [SEQ ID NO:155] |
| AGAAGGTAC | [SEQ ID NO:156] |

If the mutants do not give a clear consensus at some site, then a possible explanation is that the site is polymorphic. It is also possible that the lack of consensus is merely a statistical artifact. However, the larger the number of mutants, the more likely that a lack of consensus indicates a genuine polymorphism.

The use of SAM to detect polymorphisms is similar to existing techniques for detecting polymorphisms by shotgun sequencing (Weber and Myers, 1997; Altshuler et al., 2000). The difference is that in SAM the aligned subsequences come from mutants as well as from the original sequence. A problem with the shotgun sequencing approach is that discrepant reads can be due to either genuine polymorphisms or slightly different copies of a repeated motif. SAM can distinguish between these two possibilities. Slightly different copies of a repeated motif might be very different in one or more of the mutants, and consequently subsequences overlapping different copies of a motif can be distinguished. In such cases, a statistically significant lack of consensus can only be due to a genuine polymorphism.

3.6. Sequencing Large Sequences

SAM has the potential to sequence large stretches of sequence (e.g., megabases of DNA). There are at least two ways in which this could occur. The first is to cleave, for example, a megabase DNA molecule into smaller fragments 30-40 kb in length, each of which is then sequenced using SAM. Existing sequencing technologies involve cleaving long DNA molecules into fragments of this size, but with SAM there is an advantage in generating mutants of the long sequences prior to cleaving them into smaller pieces: mutants would then only need to be generated once at the start of the project, rather than for each individual fragment.

In a preferred embodiment, advanced SBH technology can be used to obtain hybridization spectra for very long effective probes. For example, a method of contiguous stacking probes as for example described by Stomakhin et al. (2000) may be used to obtain hybridization spectra for probes effectively eighteen bases long. This would involve hybridization of the target to a fixed array of probes of length eight, with two mobile probes of length five stacked upstream. Probes of this length, in concert with the SAM technique, may render possible the facile sequencing of very large stretches of DNA, possibly in the order of megabases.

4. Variant Subunit Sequences

4.1. Variant or Mutant Configurations

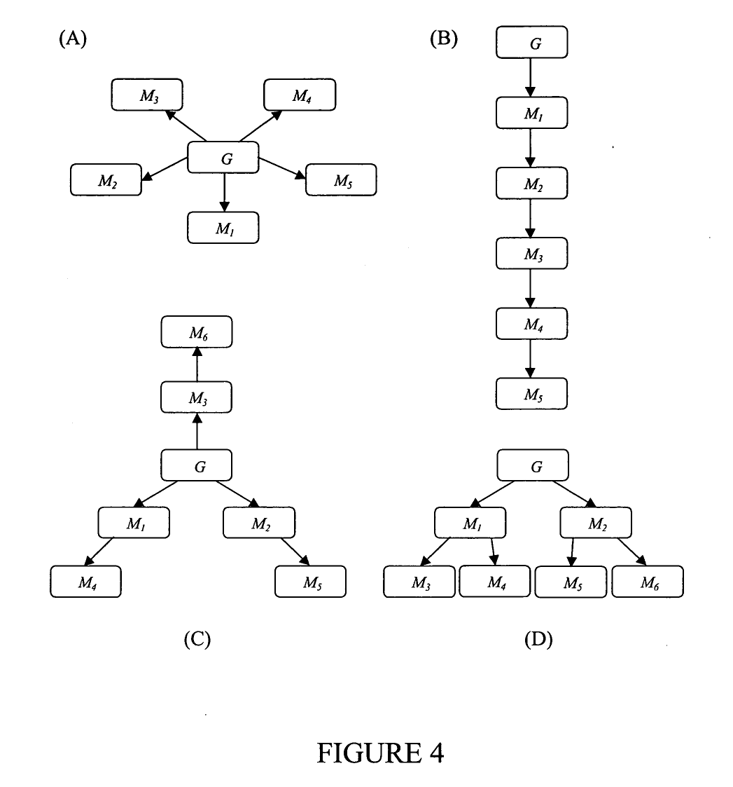

The mutants M₁, M₂, . . . , Mₙ may be related in various ways to the original sequence G. Several possible configurations are illustrated in FIG. 4. The first is the star, in which each mutant is generated directly from the original sequence. The second is the path, in which each mutant is generated from the previous mutant. The octopus and the binary tree are two generalizations, combining features of both the star and the path. If the mutants are naturally occurring (in different lineages) then they may be derived from a common ancestor of unknown sequence.

The advantage of the path configuration is that the final mutant Mₙ has a high rate of mutation relative to the original sequence G, and it is therefore highly unlikely that any of the repeats in G will appear in Mₙ. However, even though Mₙ may bear little resemblance to G, subsequences of these two sequences can be aligned by considering subsequences of the intermediate mutants. A disadvantage of the path is that it takes longer to generate mutants in this configuration because they must be generated sequentially. Another disadvantage is that a consensus of mutants in the path configuration is not a reliable estimate of the original sequence.

Generalizations such as the octopus and binary tree should combine advantages of both the star and the path.

4.2. Factors Influencing the Number of Mutants Required

Factors that influence the number of mutants required to perform a task on a target sequence, include, but are not restricted to: the intensity of mutation (proportion of sites affected); the base-specificity of mutation (some mutagens target a single base, others target all bases, but have varying preferences); the site-specificity of mutation (some mutagens target specific sites preferentially); the configuration of mutants (star, path, etc.); and the need for obtaining a consensus sequence.

In general, there are two main issues to consider when estimating the number of mutants required for SAM. The first is that there must be sufficient mutations to ensure that a substantial proportion of the problem regions in a target sequence will be rendered amenable to cloning and/or sequencing in at least one mutant. The second issue is relevant only for regions where a consensus is required: i.e., there must be a sufficient number of mutants to obtain a consensus of the desired accuracy.

With regard to the first issue, if a higher intensity of mutation is used, then fewer mutants will be required because more problem regions will be modified per mutant. However, with regard to the second issue, if a higher intensity of mutation is used, then more mutants will be required to achieve an accurate consensus sequence. Both issues must therefore be taken into account.

Generally, fewer mutants will be required if base-specific mutagenesis techniques are used to generate the mutants. This is because information about the probabilities of various substitutions can be used to obtain a more accurate alignment. Moreover, if a consensus sequence is required, it is desirable to produce a plurality of different mutants using a variety of base-specific mutagenesis techniques. Thus, sites that might have been modified in one mutant can be accurately identified in a different mutant.

To accurately calculate the number of mutants needed to achieve successful results in a particular application of SAM, detailed information regarding the base and site specificities of the mutagenesis technique would be required. The following four examples illustrate the type of calculations which might be appropriate. The first three examples consider the number of mutants needed to achieve an accurate consensus sequence. The fourth example considers the number of mutants needed to ensure that a given region is modified in at least one mutant.

First, consider a gap-closing application of SAM in which the recalcitrant target sequence and its complement can be rendered amenable to sequencing by bisulphite modification, which converts some or all cytosines to thymines. In that case, only two mutants are required, the first being a mutant of the target sequence and the second being a mutant of its complement. The first mutant is identical to the target sequence at all sites except sites where thymine appears in the mutant. Such sites may correspond to either cytosine or thymine sites in the target sequence. Alternatively, these sites may be ignored in the first mutant and accurately determined by referring to the second mutant. One can determine the complement of this second mutant in silico, thus obtaining, in effect, a mutant which differs from the target sequence only by the substitution of adenine for guanine at some sites. Cytosine and thymine sites are unaffected in this mutant, and thus may be used to complete the reconstruction. This example is important, because it demonstrates that base-specific, or even base-preferential techniques can greatly reduce the number of mutants needed to obtain accurate consensus information.

Second, suppose that the mutagenesis techniques used are neither base-specific, nor site-specific. Further suppose that the only mutations are substitutions, with all substitutions being equally likely. Then if the mutation intensity is p, and the number of mutant is n, the probability that a majority of mutants have been modified at a given site may be calculated using the binomial formula. The probability that exactly k mutants are different from the original target sequence at a given site is:

xk=(n choose k)p\hat{ }k(1−p)\hat{ }(n−k)

and the probability that the majority of mutants have been modified at that site is the sum of xk over all k greater than or equal to n/2. As an example, if 20 mutants are generated, each with a mutation intensity of 0.1, then the probability that a majority of mutants are altered at any given site is less than 1 in 100,000. This means that an incorrect or uncertain consensus will be obtained at fewer than one site in 100,000 on average under these assumptions.

Note that it is often possible to correctly determine the base appearing at a given site even when that site has been modified in a majority of mutants. The base that appears at that site in the greatest number of mutants is usually the correct one, even if it appears in fewer than half of the mutants. Thus the above method overestimates the number of mutants required to achieve an accurate consensus. Also note that it is easier to calculate the degree of accuracy given the number of mutants, than vice versa. To select the number of mutants, it might be desirable to create and use a table of consensus accuracies for various numbers of mutants and mutation intensities.

Third, suppose that the only substitutions that may occur are the four transitions A to G, G to A, C to T and T to C. Further suppose that the probability that a given A will be mutated to a G is 0.23, the probability that a given G will be mutated to an A is 0.08, the probability that a given C will be mutated to a T is 0.05, and the probability that a given T will be mutated to a C is 0.23. (These probabilities are approximately those observed in the dPTP mutants presented in Example 5). Those skilled in the art can calculate the Bayesian probabilities that the original base was an A, a C, a G or a T, given the bases observed in the relevant column of an alignment of mutants. The most probable original base can thus be determined. For example, if a G is observed at a particular site in 12 mutants, but an A is observed at that site in 8 mutants, then the Bayesian probability that the original base was an A is approximately 0.81, and the Bayesian probability that it was a G is approximately 0.19. Those skilled in the art can also determine the probability that the most probable original base according to such calculations is not the correct base. For example, if 20 mutant sequences are given, then the probability that an A will be misidentified as a G using such Bayesian calculations is approximately 0.00007. The number of mutants can thus be selected to achieve any desired accuracy.

All of the above examples focus on the number of mutations needed to obtain an accurate consensus sequence. The fourth example considers the number of mutations needed to ensure that a given problem region is sufficiently modified in at least one mutant. For example, assume that n mutants are generated, each with a mutation intensity of p. Further assume that the mutagenesis techniques used are neither base-specific nor site-specific, and that the only type of mutations are substitutions, with all substitutions being equally likely. Suppose that the problem region to be modified has length L bases, and that substitutions are required in at least r of these bases to solve the sequencing problem. Once again, the binomial formula can be used to calculate the probability that the required degree of modification occurs in at least one mutant.

The probability that exactly k substitutions occur in a given problem region of a given mutant is given by the binomial formula:

yk=(L choose k)p\hat{ }k(1−p)\hat{ }(L−k)

The probability that at least r substitutions occur in the problem region of a given mutant is y=the sum of yk over all k greater than or equal to r.

The probability that at least r substitutions occur in a given problem region of exactly m mutants is also given by the binomial formula:

zm=(n choose m)y\hat{ }m(1−y)\hat{ }(n−m)

and the probability that at least r substitutions occur in the problem region of at least one mutant is the sum of zm over all m greater than or equal to one.

From the foregoing, suppose, for example, that a problem region has length 8 bases, as in SBH with probes of length 9, where the problem regions are copies of repeats of length 8. Suppose there are 5 mutants, each with a mutation intensity of 0.3, and that it is aimed to modify the problem region in at least one base in at least one mutant. Then the probability that the region is modified in any given mutant is 0.94, and the probability that it is modified in at least one of the five mutants is 0.999999. This means that the method will fail to modify the problem region by the required amount in at least one mutant in fewer than 1 in a million cases.

It should be noted, however, that this analysis does not take into account the possibility of creating new problem regions in a mutant that were not present in the original target sequence. It should also be noted that a problem region, which is sufficiently modified in at least one mutant, does not automatically mean that the region can be correctly reconstructed using SAM. Persons of skill in the art will recognize that successful reconstruction will also rely on being able to correctly align subsequences of the mutants to each other and to the target sequence.

4.3. Methods of Producing Mutant or Variant Subunit Sequences

Any suitable mutagenesis technique for mutagenizing polymers is contemplated by the present invention. Currently, two general approaches are commonly used to mutate nucleic acids: low fidelity PCR amplification of a DNA element using conditions to promote misincorporation of nucleotides, and the chemically-induced mutagenesis of DNA followed by repair and recovery of mutants either by PCR, or by biological systems (reviewed Ling & Robertson, 1997; Leppard, 1999).

A number of different mutagenesis schemes could potentially be used to produce suitable variant sequences for use inter alia with SAM. For example, an original or parent polynucleotide can be mutated using random mutagenesis (e.g., transposon mutagenesis) or oligonucleotide-mediated (or site-directed) mutagenesis.

Oligonucleotide-mediated mutagenesis can be used for preparing suitable nucleotide substitution variants of a primary polynucleotide. This technique is well known in the art as, for example, described by Adelman et al. (1983, DNA 2:183). Briefly, a polynucleotide is altered by hybridizing an oligonucleotide encoding the desired mutation to a template DNA, wherein the template is the single-stranded form of a plasmid or bacteriophage containing the unaltered or parent DNA sequence. After hybridization, a DNA polymerase is used to synthesize an entire second complementary strand of the template that will thus incorporate the oligonucleotide primer, and will code for the selected alteration in said parent DNA sequence.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule.

The DNA template can be generated by those vectors that are either derived from bacteriophage M13 vectors, or those vectors that contain a single-stranded phage origin of replication as described by Viera et al. (1987, Methods Enzymol. 153:3). Thus, the DNA that is to be mutated may be inserted into one of the vectors to generate single-stranded template. Production of single-stranded template is described, for example, in Sections 4.21-4.41 of Sambrook et al. (1989, supra).

Alternatively, the single-stranded template may be generated by denaturing double-stranded plasmid (or other DNA) using standard techniques.

For alteration of the native DNA sequence, the oligonucleotide is hybridized to the single-stranded template under suitable hybridization conditions. A DNA polymerizing enzyme, usually the Klenow fragment of DNA polymerase I, is then added to synthesize the complementary strand of the template using the oligonucleotide as a primer for synthesis. A heteroduplex molecule is thus formed such that one strand of DNA encodes the mutated form of the polypeptide or fragment under test, and the other strand (the original template) encodes the native unaltered sequence of the polypeptide or fragment under test. This heteroduplex molecule is then transformed into a suitable host cell, usually a prokaryote such as E. coli. After the cells are grown, they are plated onto agarose plates and screened using the oligonucleotide primer having a detectable label to identify the bacterial colonies having the mutated DNA. The resultant mutated DNA fragments are then cloned into suitable expression hosts such as E. coli using conventional technology and clones that retain the desired antigenic activity are detected. Where the clones have been derived using random mutagenesis techniques, positive clones would have to be sequenced in order to detect the mutation.

Alternatively, linker-scanning mutagenesis of DNA may be used to introduce clusters of point mutations throughout a sequence of interest that has been cloned into a plasmid vector. For example, reference may be made to Ausubel et al., supra, (in particular, Chapter 8.4) which describes a first protocol that uses complementary oligonucleotides and requires a unique restriction site adjacent to the region that is to be mutagenized. A nested series of deletion mutations is first generated in the region. A pair of complementary oligonucleotides is synthesized to fill in the gap in the sequence of interest between the linker at the deletion endpoint and the nearby restriction site. The linker sequence actually provides the desired clusters of point mutations as it is moved or “scanned” across the region by its position at the varied endpoints of the deletion mutation series. An alternate protocol is also described by Ausubel et al., supra, which makes use of site directed mutagenesis procedures to introduce small clusters of point mutations throughout the target region. Briefly, mutations are introduced into a sequence by annealing a synthetic oligonucleotide containing one or more mismatches to the sequence of interest cloned into a single-stranded M13 vector. This template is grown in an E. coli dut⁻ ung⁻ strain, which allows the incorporation of uracil into the template strand. The oligonucleotide is annealed to the template and extended with T4 DNA polymerase to create a double-stranded heteroduplex. Finally, the heteroduplex is introduced into a wild-type E. coli strain, which will prevent replication of the template strand due to the presence of apurinic sites (generated where uracil is incorporated), thereby resulting in plaques containing only mutated DNA.

Methods for generating abundant mutations are preferred. Examples of such methods are based on exposing an original or target polynucleotide to mutagenizing chemicals (Leppard, 1999; Warnecke et al., 1998). The chemicals preferentially modify specific base residues or damage the base structurally. The modified DNA may then be PCR amplified, with novel nucleotide bases pairing opposite the modified bases (Fromenty et al., 2000), or recovered using in vivo repair mechanisms at a lower frequency of mutation.

Of several different chemical mutagens, bisulphite is preferred because it modifies DNA without appreciable levels of strand cleavage (Warnecke et al. 1998). Bisulphite converts the cytosines of single strand DNA into thymines, with the complementary base guanine also changing to adenine in the complementary strand (Olek et al., 1996). The fact that bisulphite induces only two types of mutation is both an advantage and a disadvantage. The advantage is that the SAM reconstruction can be made more efficient if the mutation is of a very specific nature. To see why this is so, recall that SAM is based on aligning subsequences of the original sequence to subsequences of its mutants. The number of alignments that the SAM method has to explore is greatly reduced if the only allowed mismatch is a C in the original aligned to a T in a mutant (or a G in the original aligned to an A in a mutant). The disadvantage is that this type of mutation cannot destroy a problem region (e.g., a repeated (p−1)-mer) that does not contain a C or a G. For example, a long string of A's will be preserved in all mutations induced by bisulphite treatment. Consequently, SAM will not be able to resolve reconstruction ambiguities caused by repeats of this nature. This is disadvantageous and consequently bisulphite modification alone is not sufficient for some applications of SAM (although it could be used to produce some of the desired mutations).

The chemical treatment of DNA cloned in suitable viral, BAC or YAC vectors and the subsequent in vivo recovery of mutants is also useful for the mutagenesis of larger DNA fragments (Cocchia et al., 2000). An alternative procedure involves the bisulphite-treatment of long DNA clones that may then be used to template the PCR amplification of smaller internal DNA fragments that are then cloned and sequenced by SBH.

The SAM technique can potentially be used to sequence fragments ranging in length from a few hundred bases up to an entire genome. Some of the above-mentioned mutagenesis techniques rely on PCR amplification, which is currently limited to DNA fragments of about 40 kb or shorter (Cheng et al., 1995; Fromenty et al., 2000). This is long enough to enable some exciting applications of SAM, but techniques suitable for longer fragments would greatly empower the technique, as for example described infra. The main advantage of mutating an entire genome or a large segment of a genome is that this would need to be done only once at the beginning of a sequencing project. Consequently, mutagenesis would not be a major limiting factor in the time and resource requirements of such a project.

Preferred methods of mutagenesis for use with SAM include one or more of the following: (1) DNA replication with nucleotide analogues and damaged nucleotides; (2) nucleic acid shuffling protocols based on in vitro or in vivo homologous recombination of pools of nucleic acid fragments or polynucleotides; (3) in vitro DNA replication with low fidelity polymerases and high processivity polymerases; (4) propagation of damaged DNA in repair-deficient E. coli hosts; and (5) chemical mutagens and degenerate Oligonucleotide Primer PCR. For example, these methods can be applied to two groups of DNA targets—small (1-10 kb) and large (>50 kb) DNA elements. The methods differ somewhat for the two targets, and are described infra:

Small DNA elements can be mutated by the misincorporation of bases during a nucleic acid amplification reaction which are well known to the skilled addressee, and include polymerase chain reaction (PCR) as for example described in Ausubel et al. (supra); strand displacement amplification (SDA) as for example described in U.S. Pat. No. 5,422,252; rolling circle replication (RCR) as for example described in Liu et al., (1996) and International application WO 92/01813) and Lizardi et al., (International Application WO 97/19193); nucleic acid sequence-based amplification (NASBA) as for example described by Sooknanan et al., (1994); and Q-â replicase amplification as for example described by Tyagi et al., (1996). In a preferred embodiment, such mutagenesis is carried out using PCR-directed mutagenesis. In this method, small DNA elements can be mutated efficiently (1-20%.) by using non-standard base analogues (Kamiya et al., 1994 Nucleosides and Nucleotides 13: 1483-1492; Zaccolo et al., 1996 Journal of Molecular Biology 255: 589-603), or less efficiently by limiting the provision of some bases (Cline et al., 1996 Nucleic Acids Research 24: 3546-3551), or by chemically reducing polymerase fidelity (Rice et al., 1992 Proceedings of the National Academy of Science USA 89: 5467-5471; Vartanain et al., 1996 Nucleic Acids Research 24: 2627-2631; Shafikhani et al., 1997 Biotechniques 23: 304-310). As will be appreciated by those of skill in the art, particular nucleotide analogues are incorporated by Taq DNA polymerase and cause a known range of mutations. For example, dPTP [6-(2-deoxy-B-D-ribofuranosyl)-3,4-dihydro-8H-pyrimido-[4,5-C]oxazin-7-one triphosphate] induces A->G and T->C transitions, while 8-oxo-dGTP preferentially causes A->C and T->G transversions (Zaccolo et al., 1996 Journal of Molecular Biology 255: 589-603). Other nucleoside analogues, such as N⁶-methoxy-2,6-diaminopurine (dK) and N⁶-methoxyoxyaminopurine (dZ) (Hill et al., 1998a Nucleic Acids Research 26: 1144-1149: Hill et al., 1998b Proceedings of the National Academy of Science USA 95: 4258-4263) also induce particular mutations. As high levels of modification can introduce mismatches between the specific PCR primers and the modified template, short discriminatory primers can be used to recover specific products (Mitchelson et al., 1999 Nucleic Acids Research [Methods on Line] 27:e28). Polymerase co-elements that aid processivity during PCR can also be used to increase the attainable size of amplified products (Motz et al., 2002 Journal of Biological Chemistry published January 22 as 10.1074/jbc.M107793200).

Small DNA elements can also be mutated using recombination techniques, as for example disclosed by Stemmer in U.S. Pat. Nos. 6,344,356, 6,323,030, 6,297,053, 6291,242, 6,297,861, 6,277,638, 6,180,406, 6,165,793 and 6,117,679, which employ repeated cycles of mutagenesis, shuffling and selection, and which allow for the directed molecular evolution in vitro or in vivo of nucleic acid sequences. In this method, mixtures of related nucleic acid sequences or polynucleotides are randomly fragmented, and reassembled to yield a library or mixed population of recombinant nucleic acid molecules or polynucleotides.

A mutant DNA polymerase with lowered fidelity for incorporation of correct complementary nucleotides during DNA synthesis, and which is preferably thermostable, is preferably employed in such nucleic acid amplification-directed mutagenesis protocols. For example, a mutant Taq polymerase has been found to produce significant levels of random mutation during PCR amplification (U.S. Pat. No. 6,329,178; Suzuki et al, 1997 Journal of Biological Chemistry 272: 11228-11235). This mutant polymerase can also incorporate nucleotide analogues as efficiently or more efficiently than native Taq polymerase. In a preferred embodiment, rounds of mutagenesis with the low-fidelity polymerase and nucleotide analogues are used to effect modification in genomic sub-fragments and other small DNAs. The length of DNA that can be mutated exhaustively is only limited by the PCR procedure, which can routinely amplify 10-20 kb fragments, aided by E. coli exonuclease III (Fromenty et al., 2000 Nucleic Acids Research [Methods on Line] 28:e50) and other protein factors (Motz et al, 2002 supra).

Bacterial strains, which are deficient in enzymes of excision repair pathways that catalyze different steps in DNA sanitation, are preferably employed and these are well known to practitioners versed in the art. Examples include E. coli strains that fail to remove oxidative damaged and deaminated nucleotides efficiently, post-replication (Miller 1992, in A Short Course in Bacterial Genetics, CSH Press; Yonezawa et al., 2001 Mutation Research 490:21-26; Kamiya and Kasai, 2000 Nucleic Acids Research 28:1640-1646). In a preferred embodiment, DNA and nucleotide analogues, as for example described above, are co-transfected into repair-deficient bacteria, which results in increased levels of mutation, as mispaired bases are not thoroughly removed (Inoue et al., 1998 Journal of Biological Chemistry 273: 11069-11074; Fujikawa et al., 1998 Nucleic Acids Research 26: 4582-4587). The co-transfection of nucleotide analogues and DNAs into repair-deficient host strains can also be used to mutate random shotgun libraries at low mutation frequencies.

Larger DNA elements can be mutated efficiently using nucleotide analogues and repair-deficient bacteria. For example, nucleotide analogues and larger DNA elements such a BACs can be co-transfected into repair-deficient host strains to generate mutant BACs. The in vivo functionality of the modified BACs may be recovered efficiently in E. coli by homologous recombination (Nefedov et al., 2000 Nucleic Acids Research [Methods on Line] 28:e79).

Alternatively, rolling circle amplification (RCA) can be employed for mutating larger DNA elements. RCA polymerase including Phi29 DNA polymerase and other polymerases permit the synthesis of large circular dsDNA molecules such a large plasmids and BACs (Dean et al., 2001 Genome Research 11: 1095-1099; Amersham Biosciences, 2002 TempliPhi, Amersham Technical note; Zhang et al., 2001 Gene 274: 209-216). The ability to replicate large DNAs in vitro permits mutation to higher levels, without the functional limits imposed by replication in bacterial hosts. In accordance with the present invention, this technique is used in concert with nucleotides such as dPTP and other deoxynucleotide triphosphate analogues to incorporate these analogues directly into DNA templates. Clones harboring mutant DNAs can then be recovered in a suitable host (e.g., E. coli) by homologous recombination.

Larger DNA elements can also be mutated advantageously using RNA polymerase amplifications. In this context, the high processivity of RNA polymerases and RNA reverse transcriptases can be used to amplify DNA fragments (Iwata et al., 2000 Bioorganic and Medical Chemistry 8: 2185-2194; Bebenek et al., 1999 Mutation Research 429: 149-158) with incorporation of ribo-nucleotide or deoxynucleotide analogues. Ribo-nucleotide analogues are mutagenic and some are incorporated into both RNA (U.S. Pat. No. 6,132,776; U.S. Pat. No. 5,512,431; Moriyama et al., 1998 Nucleic Acids Research 26: 2105-2111) and DNA (Miller et al., 1978 Journal of Molecular Biology 124: 343-358), and deoxynucleotide analogues are incorporated by reverse transcriptase into DNA (Lutz et al., 1998 Bioorganic and Medical Chemical Letters 8: 499-504; Bebenek et al., 1999 Mutation Research 429: 149-158). RNA products incorporating ribonucleotide analogues can be copied from cloned DNAs residing in suitable plasmid vectors possessing RNA polymerase promoters. The RNA products can be used to create mutated cDNA, which are then subsequently sub-cloned and sequenced individually.

Chemical mutagens can also be used advantageously to mutate larger DNA elements. For example, chemicals such as nitrous acid, glyoxal, bisulphite, peroxide, hydrazine and other mutagenic agents modify several different base residues, or damage the base structurally (Burney et al., 1999 Mutation Research 424: 37-49; Wagner et al., 1992 Proceedings of the National Academy of Science USA 89: 3380-3384; Rodriguez et al., 1999 Biochemistry 38: 16578-16588; Murata-Kamiya et al., 1997 Mutation Research 377: 13-16). The damaged DNA can be recovered by in vivo recovery of the target in plasmid or BAC vectors (Ling and Robinson, 1997 Analytical Biochemistry 254: 157-178; Leppard, 1999 in DNA Viruses: A Practical Approach. vol 214, IRL Press) with low-level chemical modification.

The present invention also contemplates whole genome mutagenesis. Several routes to random mutation of whole genomes are known, and these generally fall into two major categories: (i) induced-mutagenesis in biological systems or whole cell lines and (ii) (low fidelity) PCR amplification and replication of DNA elements using conditions to promote misincorporation of nucleotides, or analogues of nucleotides.

Induced mutagenesis can carried out by methods which include, but are not limited to, whole cell mutation, large cloned element mutagenesis, degenerate oligonucleotide primer PCR and shotgun mutagenesis.

Whole cell mutation involves the induction of mutation in stable cell lines from an organism, or in hybrid cells lines that carry an individual chromosome from the organism under study within the cell of another organism. The advantage of this approach is the potential to isolate individual mutant cell lines that may be used as a recurrent source of a particular mutated DNA sequence, while retaining the larger chromosomal context of that sequence.

Large cloned element mutagenesis that has been used in the sequencing of the human genome (International Human Genome Sequencing Consortium 2001, Nature 409: 860-921), and the genomes of many other organisms. In this procedure, the genome is subcloned into a set of overlapping fragments of 100-200 kb supported in BAC and other large element vectors. A minimum overlapping set of these large elements that represent the genome is then further sub-cloned into plasmids (1-3 kb inserts). The plasmid-contained genomic DNA elements are then sequenced. Mapping and fingerprinting techniques, such as BAC insert end-sequencing, restriction fingerprinting, STR fingerprinting, hybridizations with cDNA and other cloned and sequenced DNA elements, as well as cross-hybridization between BAC elements is used to identify the genomic elements and to create contigs of the overlapping large cloned elements. Mutation of the genome can be performed segmentally on the large element clones preferably using methods for mutagenesis of large DNA elements, as for example described supra. (Low fidelity) PCR amplification can carried out by methods which include, but are not restricted to, degenerate oligonucleotide primer PCR and shotgun mutagenesis.

Random amplification by degenerate oligonucleotide primer (DOP)PCR can be used to recover essentially random DNA fragments of 0.5 kb to 2 kb from limiting amounts of genomic DNA and from individual cytometric flow-sorted chromosomes (Zhou et al., 2000 Biotechniques. 2: 766-767; Hirose et al., 2001 Journal of Molecular Diagnostics 3: 62-67). Nucleotide analogues are incorporated efficiently by these fragment sizes by PCR to as high as ˜20% mutation. DOP-PCR can used to amplify from whole genomes, individual chromosomes, or to amplify from large DNA fragments such as cloned BACs to limit the sequence complexity. Such random amplified mutant fragments can then be sub-cloned to form a representative mutant library.

Shotgun cloning of entire genomes has also been used in the sequencing the human genome (Venter et al., 2001 Science 291: 1304-1351). The method involves the subcloning and DNA sequencing of a selection of randomly broken, short, overlapping DNA elements that collectively represent the original genome, and the reconstruction of the original sequence by the computer-aided alignment of the resulting multiple overlapping sequence reads. This principle could be applied to the cloning of chemically-modified DNA, in which nucleotide-damage internal to the random fragments will result in recovery of a mutated shotgun clone library.

Chemical modification of DNA can be achieved by several different methods. For example random chemical modification of nucleotide bases (with attendant double strand and single strand breakage of the genome into smaller fragments) can be used for shotgun-mutagenesis. Preferably, such chemical modification is combined with processes for efficient fragment end-repair and sub-cloning of the damaged DNAs. End repair enzymes such as E. coli endonuclease IV (Levin et al 1988 Journal of Biological Chemistry 263:8066-8071; Demple and Harrison 1994 Annual Review Biochemistry 63:915-948) and endonuclease III (Masson and Ramotar, 1997 Molecular Microbiology 24:711-721) are used to remove 3′-phosphoglycolates and different 3′-phosphates that may arise at the termini of chemically broken DNA fragments, and additionally conventional DNA polymerases (such as Klenow enzyme or T4 DNA polymerase) and polynucleotide kinases (e.g., T4 polynucleotide kinase) are used to ‘fill-out’ single strand fragment termini and to phosphorylate 5′-termini, respectively. Unless repaired, together the ragged and blocked DNA fragment termini would prevent ligation of the fragment into the plasmid vector. This method of introducing mutagenesis into genomic DNA prior to subcloning fragments has the implicit advantage that DNA fragments, which may possess sequence motifs that prevent convenient cloning of the element, may be altered or disrupted by the mutation and hence may be cloned and represented within the library. Lower levels of chemical modification of genomic DNA may also be sought prior to the shotgun cloning to provide DNA fragments which are more intact and which need to be sheared mechanically to produce 2-4 kb fragments suitable for inclusion in the random library. End repair of such sheared fragments are readily achieved and subsequent cloning of these fragments is efficient.

Chemical modification of DNA can also be achieved by conventional shotgun cloning followed by subsequent mutation of the random genomic sub-fragments. The subsequent mutation may be carried out by library mutagenesis, or individual sub-clone mutagenesis, which has the advantage that subclones of genomic DNA that are created may be first created and cloned efficiently without chemical damage to the termini requiring particular repair steps. Library-mutagenesis is suitably achieved either by the above methods for small element mutagenesis in which the entire random representative library is subjected to a mutagenic procedure and subsequently, random mutant clones are chosen from the resultant library, or collections of clones from the random library, e.g., 96 clones, are collectively mutated by nucleotide analogue PCR and the resultant amplicons are re-cloned to make a sub-set mutant library that can be conveniently related back to the original unmodified 96 clones.

If biological systems are used for the mutation and the recovery stages as described above as applied to small element mutagenesis, lower levels of mutation might be necessary to preserve biological functions of the vector and host cells. If a general PCR-based mutagenic procedure is used on the whole shotgun library, techniques to achieve high levels of mutation of the amplified mutant library elements such as the incorporation of nucleotide analogues could be employed, and the mutant elements again sub-cloned into plasmids to produce a random, highly-mutant fragment library. The same mutagenic methods may be employed for individual shotgun clones or with small collections of clones so as to minimize the sequence complexity of the resultant mutant library or libraries.

In a preferred method, which may avoid the loss of DNA elements that are otherwise difficult to clone in conventional plasmid vectors and host E. coli (including low copy plasmids, read-through truncated plasmids, and E. coli hosts cells lacking restriction and/or recombination functions), short, sheared random genomic DNA elements are ligated into new plasmid vectors, which are directly used as a random template for a PCR-based nucleotide analogue mutation protocol to generate amplicons from the total library, before the loss of potentially unclonable elements through cloning in E. coli. The oligonucleotide primers for PCR are preferably complementary to vector sequences flanking the ligated elements and possess (rare) restriction sites that are either all C:G or A:T. Nucleotide analogues that target either A:T or C:G base-pairs for sequence mutation are then employed, which leave one of the two types of restriction sites unaltered and thus available for convenient regeneration of restriction termini for cloning of the amplicons into the new plasmid vectors. In this manner, a fully representative genome library could be efficiently mutated before passage through E. coli cells.

Preferred hosts and/or vectors for cloning parent or mutagenized sequences are those which have been engineered to ameliorate difficulties in cloning otherwise difficult-to-clone nucleic acid molecules. For example, bacterial strains, particularly strains of E. coli, and engineered plasmid vectors are known to practitioners in the art, which have been selected or engineered to overcome such difficulties. Exemplary strains for this purpose include, but are not restricted to: E. coli strains engineered to limit recombination of DNA, such as JM110 cells that accept repetitive DNA, as for example disclosed by Troester et al. (2000, Gene 258: 95-108), E. coli strains engineered to be methylation-tolerant (mcrA⁻ mcrB⁻) that limit the restriction of unmethylated or ‘incorrectly’ methylated DNA and thus accept mammalian DNA-containing clones, as for example disclosed by Doherty et al. (1991, Gene 98: 77-82) and Williamson et al. (1993, Gene 124: 37-44; Stratagene Corp., SURE cells); and E. coli strains engineered to be deficient in DNA sanitation enzyme(s) that promote the mutation of introduced DNA as for example described by Deng and Nickoloff (1992, Analytical Biochemistry 200: 81-88), Greener and Callahan (1994, Strategies 7: 32-34), Cox and Homer (1986, Journal of Molecular Biology 190: 113-117) and Miller (1992, in A Short Course in Bacterial Genetics, CSH Press). Suitable plasmids include, but are not limited to: plasmid vectors that have been engineered to prevent read-through transcription (e.g., the CloneSmart™ vector system from Lucigen Corporation, Middleton Wis. 53562, USA, which is a gap-free cloning system available for sequencing recalcitrant or unclonable DNA), low copy plasmids that replicate in E. coli hosts to 1-10 copies per cell in which repeat DNA elements may be maintained (e.g., pBR_{III} and its derivatives as for example described by Mitchelson and Moss, 1987 Nucleic Acids Research 15: 9577-9596; and pEV-vrf3 as for example described by Perng et al., 1994 Journal of Virological Methods 46: 111-116).

It will be understood that SAM can be applied to the sequencing of any ‘problematic’ polymer including, for example, polypeptides and carbohydrates. Variant or mutant polypeptides can be produced using any suitable technique. For example, mutant polypeptides may be produced from mutant polynucleotides prepared by rational or random mutagenesis methods as, for example, described supra.

Sequencing of a polypeptide may be performed by site-directed or random cleavage of the polypeptide using, for example endopeptidases or CNBr, to produce a set of polypeptide fragments and subsequent sequencing of the polypeptide fragments by, for example, Edman sequencing or mass spectrometry, as is known in the art. Alternatively, the polypeptide probes or polypeptide fragments could be sequenced by use of antibody probes as for example described by Fodor et al in U.S. Pat. No. 5,871,928. Briefly, such antibody probes specifically recognize particular subsequences (e.g., at least three contiguous amino acids) found on a polypeptide. Optimally, these antibodies would not recognize any sequences other than the specific desired subsequence and the binding affinity should be insensitive to flanking or remote sequences found on a target molecule.

5. Sequencing by SBH and SAM

The invention also provides a method for extending an incomplete reconstruction of a primary nucleic acid sequence by comparing overlapping subsequences, of length p, corresponding to said primary nucleic acid sequence, wherein said reconstruction is incompletable due to the presence of repeated subsequences of length p−1 in said primary nucleic acid sequence. The method comprises exposing an array of oligonucleotide probes comprising a sequence of length p, under stringent hybridization conditions, to at least one secondary nucleic acid sequence which varies from the primary nucleic acid sequence by the addition, deletion and/or substitution of at least one nucleotide, wherein said variation is associated with the alteration or destruction of at least one of said repeated subsequences. Hybridization data is then processed to detect which of said probes have hybridized to said at least one secondary nucleic acid sequence and to thereby determine a set of subsequences, of length p, corresponding to said at least one secondary nucleic acid sequence. Overlapping subsequences, of length p, corresponding to said at least one secondary nucleic acid sequence and to said primary nucleic acid sequence, are then compares to unambiguously extend the incomplete reconstruction.

In a preferred embodiment, the probes are immobilized on one or more solid supports. An oligonucleotide probe may be immobilized to the solid support using any suitable technique. Preferably, the probes are in the form of a nucleic acid array, preferably a high-density nucleic acid array.

Probes may be designed to optimize specific hybridization to their reference sequences. For example, Drmanac et al. (U.S. Pat. No. 5,972,619) describe probes containing a core 8-mer and one of three possible variations at outer positions with two variations at each end. Such probes are represented as 5′-(A, T, G, C)(A, T, G, C)N8 (A, T, G, C)-3′. With this type of probe one does not need to discriminate the non-informative end bases (two on 5′ end, and one on 3′ end) since only the internal 8-mer is read as the probe sequence.

The variant or secondary nucleic acid sequence referred to above is potentially a target polynucleotide for the above set of probes and ii includes, but is not restricted to, DNA or RNA. Sample extracts of DNA or RNA, either single or double-stranded, may be prepared from fluid suspensions of biological materials, or by grinding biological materials; or following a cell lysis step which includes, but is not limited to, lysis effected by treatment with SDS (or other detergents), osmotic shock, guanidinium isothiocyanate and lysozyme. Suitable DNA, which may be used in the method of the invention, includes genomic DNA or cDNA. Such DNA may be prepared by any one of a number of commonly used protocols as for example described in CURRENT PROTOCOLS IN MOLECULAR BIOLOGY (Ausubel, et al., eds.) (John Wiley & Sons, Inc. 1995), and MOLECULAR CLONING. A LABORATORY MANUAL (Sambrook, et al., eds.) (Cold Spring Harbor Press 1989). Sample extracts of RNA may be prepared by any suitable protocol as for example described in CURRENT PROTOCOLS IN MOLECULAR BIOLOGY (supra), MOLECULAR CLONING. A LABORATORY MANUAL (supra) and Chomczynski and Sacchi (1987, Anal. Biochem. 162 156, hereby incorporated by reference).

Suitable RNA, which may be used in the method of the invention, includes messenger RNA, complementary RNA transcribed from DNA (cRNA) or genomic or subgenomic RNA. Such RNA may be prepared using standard protocols as for example described in the relevant sections of Ausubel, et al. (supra) and Sambrook, et al. (supra).

The genomic DNA or cDNA may be fragmented, for example, by sonication or by treatment with restriction endonucleases. Suitably, the genomic DNA or cDNA is fragmented such that resultant DNA fragments are of a length greater than the length of the immobilized oligonucleotide probe(s) but small enough to allow rapid access thereto under suitable hybridization conditions. Alternatively, fragments of genomic DNA or cDNA may be amplified using a suitable nucleotide amplification technique, involving appropriate random or specific primers. Such amplification techniques are well known to those of skill in the art and include, for example, PCR (Saiki et al, 1988, supra), Strand Displacement Amplification (SDA) (U.S. Pat. No. 5,422,252, Little et al.), Rolling Circle Replication (RCR) (Liu et al., 1996, J. Am. Chem. Soc. 118 1587-1594; International Application Publication No WO 92/01813), Nucleic Acid Sequence Based Amplification (NASBA) (Sooknanan et al., 1994, Biotechniques 17 1077-1080) and Q-â replicase amplification (Tyagi et al., 1996, Proc. Natl. Acad. Sci. USA 93 5395-5400).

Usually the target polynucleotide is detectably labeled so that its hybridization to individual probes can be determined. In this regard, the target polynucleotide may have one or more reporter molecules associated therewith. The reporter molecule may be selected from a group including a chromogen, a catalyst, an enzyme, a fluorochrome, a chemiluminescent molecule, a bioluminescent molecule, a lanthamide ion such as Europium (Eu³⁴), a radioisotope and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like. Especially preferred labels of this type include large colloids, for example, metal colloids such as those from gold, selenium, silver, tin and titanium oxide. In one embodiment in which an enzyme is used as a direct visual label, biotinylated bases are incorporated into a target polynucleotide. Hybridization is detected by incubation with streptavidin-reporter molecules.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al. (International Publication WO 93/06121). Reference also may be made to the fluorochromes described in U.S. Pat. No. 5,573,909 (Singer et al), U.S. Pat. No. 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218. Commercially available fluorescent labels include, for example, fluorescein phosphoramidites such as Fluoreprime (Pharmacia), Fluoredite (Millipore) and FAM (Applied Biosystems International).

Radioactive reporter molecules include, for example, ³²P, which can be detected by a X-ray or phosphoimager techniques.

The hybrid-forming step can be performed under suitable conditions for hybridizing oligonucleotide probes to test nucleic acid including DNA or RNA. In this regard, reference may be made, for example, to NUCLEIC ACID HYBRIDIZATION, A PRACTICAL APPROACH (Homes and Higgins, eds.) (IRL press, Washington D.C., 1985). In general, whether hybridization takes place is influenced by the length of the oligonucleotide probe and the polynucleotide sequence under test, the pH, the temperature, the concentration of mono- and divalent cations, the proportion of G and C nucleotides in the hybrid-forming region, the viscosity of the medium and the possible presence of denaturants. Such variables also influence the time required for hybridization. The preferred conditions will therefore depend upon the particular application. Such empirical conditions, however, can be routinely determined without undue experimentation.

Preferably high discrimination hybridization conditions are used. For example, reference may be made to Wallace et al. (1979, Nucl. Acids Res. 6: 3543) who describe conditions that differentiate the hybridization of 11 to 17 base long oligonucleotide probes that match perfectly and are completely homologous to a target sequence as compared to similar oligonucleotide probes that contain a single internal base pair mismatch. Reference also may be made to Wood et al. (1985, Proc. Natl. Acid. Sci. USA 82: 1585) who describe conditions for hybridization of 11 to 20 base long oligonucleotides using 3M tetramethyl ammonium chloride wherein the melting point of the hybrid depends only on the length of the oligonucleotide probe, regardless of its GC content. In addition, Drmanac et al. (supra) describe hybridization conditions that allow stringent hybridization of 6-10 nucleotide long oligomers.

Generally, a hybridization reaction can be performed in the presence of a hybridization buffer that optionally includes a hybridization optimizing agent, such as an isostabilizing agent, a denaturing agent and/or a renaturation accelerant. Examples of isostabilizing agents include, but are not restricted to, betaines and lower tetraalkyl ammonium salts. Denaturing agents are compositions that lower the melting temperature of double stranded nucleic acid molecules by interfering with hydrogen bonding between bases in a double stranded nucleic acid or the hydration of nucleic acid molecules. Denaturing agents include, but are not restricted to, formamide, formaldehyde, dimethylsulphoxide, tetraethyl acetate, urea, guanidium isothiocyanate, glycerol and chaotropic salts. Hybridization accelerants include heterogeneous nuclear ribonucleoprotein (hnRP) A1 and cationic detergents such as cetyltrimethylammonium bromide (CTAB) and dodecyl trimethylammonium bromide (DTAB), polylysine, spermine, spermidine, single stranded binding protein (SSB), phage T4 gene 32 protein and a mixture of ammonium acetate and ethanol. Hybridization buffers may include target polynucleotides at a concentration of between about 0.005 nM and about 50 nM, preferably between about 0.5 nM and 5 nM, more preferably between about 1 nM and 2 nM

A hybridization mixture containing the target polynucleotide is placed in contact with the array of probes and incubated at a temperature and for a time appropriate to permit hybridization between the target sequences in the target polynucleotide and any complementary probes. Contact can take place in any suitable container, for example, a dish or a cell designed to hold the solid support on which the probes are bound. Generally, incubation will be at temperatures normally used for hybridization of nucleic acids, for example, between about 20 C and about 75 C, example, about 25 C, about 30 C, about 35 C, about 40. C, about 45 C, about 50 C, about 55 C, about 60 C, or about 65 C. For probes longer than 14 nucleotides, 20 C to 50 C is preferred. For shorter probes, lower temperatures are preferred. A sample of a target polynucleotide is incubated with the probes for a time sufficient to allow the desired level of hybridization between the target sequences in the target polynucleotide and any complementary probes. For example, the hybridization may be carried out at about 45° C.+/−10° C. in formamide for 1-2 days.

After the hybrid-forming step the probes are washed to remove any unbound nucleic acid with a hybridization buffer, which can typically comprise a hybridization optimizing agent in the same range of concentrations as for the hybridization step. This washing step leaves only bound target polynucleotides. The probes are then examined to identify which probes have hybridized to a target polynucleotide.

The hybridization reactions are then detected to determine which of the probes has hybridized to a corresponding target sequence. Depending on the nature of a reporter molecule associated with a target polynucleotide, a signal may be instrumentally detected by irradiating a fluorescent label with light and detecting fluorescence in a fluorimeter; by providing for an enzyme system to produce a dye which could be detected using a spectrophotometer; or detection of a dye particle or a colored colloidal metallic or non metallic particle using a reflectometer; in the case of using a radioactive label or chemiluminescent molecule employing a radiation counter or autoradiography. Accordingly, a detection means may be adapted to detect or scan light associated with the label which light may include fluorescent, luminescent, focussed beam or laser light. In such a case, a charge couple device (CCD) or a photocell can be used to scan for emission of light from a probe:target polynucleotide hybrid from each location in the micro-array and record the data directly in a digital computer. In some cases, electronic detection of the signal may not be necessary. For example, with enzymatically generated color spots associated with nucleic acid array format, as herein described, visual examination of the array will allow interpretation of the pattern on the array. In the case of a nucleic acid array, the detection means is preferably interfaced with pattern recognition software to convert the pattern of signals from the array into a plain language genetic profile. In a preferred embodiment, the set of probes is in the form of a nucleic acid array and detection of a signal generated from a reporter molecule on the array is performed using a ‘chip reader’. A detection system that can be used by a ‘chip reader’ is described for example by Pirrung et al (U.S. Pat. No. 5,143,854). The chip reader will typically also incorporate some signal processing to determine whether the signal at a particular array position or feature is a true positive or maybe a spurious signal. Exemplary chip readers are described for example by Fodor et al (U.S. Pat. No., 5,925,525).

The hybridization data obtained from the above hybridization reactions are processed to determine which probes have formed hybrids, wherein the probes detect specifically individual target sequences under stringent hybridization conditions. In a preferred embodiment, a processing means is employed to correlate specific positional labeling on the array with the presence of any of the target sequences for which the probes have specificity of interaction. Typically, the positional information is directly converted to a database, indicating what sequence interactions have occurred. Data generated in hybridization assays is most easily analyzed with the use of a processing system such as but not limited to a programmable digital computer. Any general or special purpose processing system is contemplated by the present invention, as for example described infra. In one embodiment, certain files are devoted to memory that includes the location of each feature and all the target sequences known to contain the sequence of the oligonucleotide probe at that feature. Computer-implemented methods for analyzing hybridization data from nucleic acid arrays is taught in PCT publication No WO97/29212 and EP publication 95307476.2.

Thus, once the probes, which have hybridized to the target polynucleotide, have been detected, a set of subsequences is deduced corresponding to the target polynucleotide, which are complementary to those probes. Comparison of this set of subsequences to a previously determined set of subsequences corresponding to the primary or original polynucleotide is then carried out according to the method described in Section 3.2 to extend the incomplete reconstruction of the primary polynucleotide.

6. Computer-Related Embodiments

The present invention discloses methods for sequence analysis, which may be conveniently implemented by a processing system such as a computer system. These methods are predicated in part on the provision or generation of data representing variant subunit sequences, which are distinguished individually from a target subunit sequence by the addition, deletion and/or substitution of at least one subunit. The ready use of these data preferably, but not essentially, requires that they be stored in a format that is usable by a processing system which is adapted to generate or deduce, on the basis of those data, all or part of the target subunit sequence. Thus, in accordance with one embodiment of the present invention, data representing subunit sequences as described above may be stored in a data store, which preferably includes a database, for use by a processing system in operable communication with the data store. The data store may have stored therein the full-length subunit sequences or may comprise portions or sub-sequences of those sequences.

Suitably, the processing system is adapted to process the data in the data store to generate a comparison of the individual sequences and optionally of a sequence derived from, or adjacent to, the target subunit sequence. The processing of the data may also include deducing a consensus sequence from the comparison, which corresponds to all or part of the target subunit sequence.

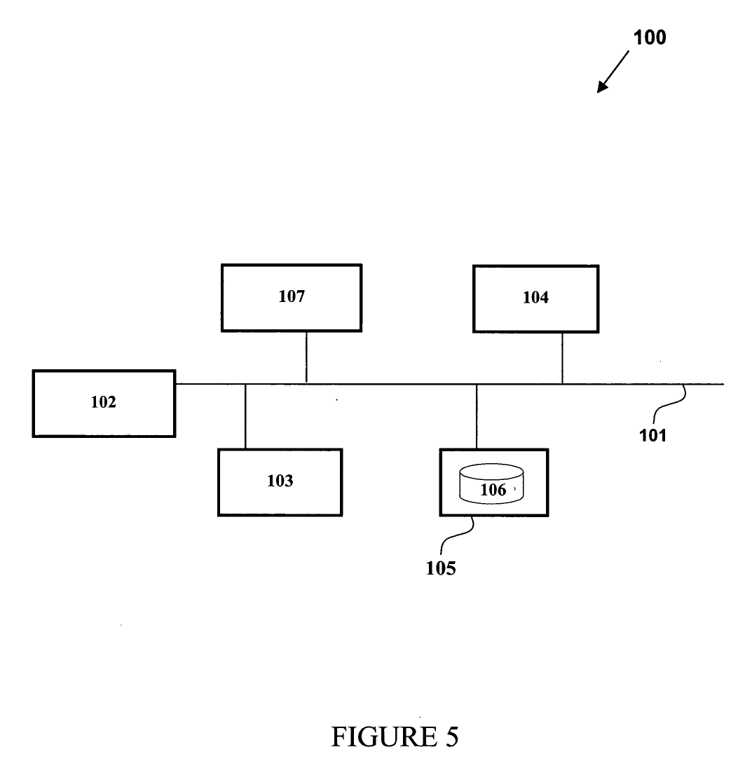

Any general or special purpose processing system is contemplated by the present invention and includes, but is not limited to, a processor in operable (e.g., electrical) communication with both a memory and at least one input/output device, such as a keyboard and a display. Such a system may include, but is not limited to, personal computers, workstations or mainframes. The processor may be a general purpose processor or microprocessor or a specialized processor executing programs located in RAM memory. The programs may be placed in memory (e.g., RAM) from a storage device, such as a disk or pre-programmed ROM memory. The RAM memory in one embodiment is used both for data storage and program execution. The processing system also embraces systems where the processor and memory reside in different physical entities but which are in operable communication by means of a network. For example, a processing system having the overall characteristics set forth in FIG. 5 may be useful in the practice of the instant invention. More specifically, FIG. 5 is a schematic representation of a processing system (100) having in operable communication (101) with one another via, for example, an internal bus or external network, a processor (102), a memory (103), an input/output device (104) such as a keyboard and display and a data store (105), which typically includes a database (106). For example, the data store may be in the form of an external storage device such as but not limited to a diskette, CD ROM, or magnetic tape. It will, therefore, be appreciated that the processing system 100 may be formed from any suitable processing system, which is capable of operating applications software to enable the processing of the data, such as a suitably programmed personal computer.

When in electrical communication with an external network, the processing system (100) will preferably be formed from a server, such as a network server, web-server, or the like allowing the analysis to performed from remote locations. In this case, the processing system includes an interface (107), such as a network interface card, allowing the processing system to be connected to remote processing systems, such as via the Internet as will be described in more detail below.

In the practice of one embodiment of the present invention, the processing system executes a sequence analysis program that includes computer executable code which when implemented on the processing system causes the system to receive the sequences of a plurality of variants and optionally a sequence derived from, or adjacent to, the target subunit sequence, as described above. The sequences may be obtained from a number of sources, such as manual input via the I/O device 104 or received from an external processing system via the interface 107; or by accessing subunit sequences stored in the database 106. The system is also caused to align the individual sequences of the variants to each other and optionally to the sequence derived from, or adjacent to, the target subunit sequence to produce a set of aligned sequences. The aligned sequences are then compared to each other and a consensus sequence is deduced from this comparison which corresponds to all or part of the target subunit sequence. In the comparison step, the system preferably compares the subunit at each position in a subunit sequence under test to the subunit at an identical position in other subunit sequences to thereby determine any matches between the subunit sequence under test and any of the other subunit sequences, as well as any differences therebetween. From this comparison, a consensus of the variant subsequences can be deduced as for example described in Section 3. Optionally the processing means analyzes base statistics at each site to determine the quality of the consensus, and to identify possible sites at which there are sequencing errors or polymorphisms.

In a preferred embodiment, the processing system is further adapted to generate an indication of the target subunit sequence, which is preferably displayed by a display means that is part of the processing system.

In the practice of another embodiment of the present invention, the processing system executes a program for unambiguously extending an incomplete reconstruction of a primary subunit sequence by comparing overlapping subsequences corresponding to the primary subunit sequence, wherein the reconstruction is incompletable due to the presence of repeated subsequences in the primary subunit sequence. In this instance, the program includes computer executable code which when implemented on the processing system causes the system to receive data (e.g., from a data store or database) representing at least one secondary subunit sequence which varies from the primary subunit sequence by the addition, deletion and/or substitution of at least one subunit, wherein the variation is associated with the alteration or destruction of at least one of the repeated subsequences. The computer executable code also causes the processing system to compare overlapping subsequences corresponding to the secondary subunit sequence(s) and to the primary subunit sequence, to unambiguously extend the incomplete reconstruction and to thereby form an extended reconstruction. The individual overlapping subsequences preferably have a length p and the repeated subsequences preferably have a length p−1.

In a preferred embodiment, the processing system is further caused to alternately reconstruct the primary subunit sequence and secondary subunit sequence(s) using an end portion of a respective reconstruction as a guide to extend another reconstruction. The processing system suitably carries out this reconstruction by comparing an end portion of the incomplete reconstruction with subsequences corresponding to the secondary subunit sequence(s) to identify a subsequence which aligns best with the end portion and which extends unambiguously in the alignment a reconstruction of the secondary subunit sequence(s) beyond the incomplete reconstruction of the primary subunit sequence to form an extended reconstruction of the secondary subunit sequence(s). An end portion of the extended reconstruction is then compared by the processing system with subsequences corresponding to the primary subunit sequence to identify a subsequence which aligns best with said end portion of the extended reconstruction and which extends unambiguously in the alignment the incomplete reconstruction of the primary subunit sequence to thereby form an extended reconstruction of the primary subunit sequence. Preferably, the processing system deduces a best alignment between a subsequence and an incomplete reconstruction by comparing the alignment of different subsequences with the incomplete reconstruction to produce a plurality of extended reconstructions together with individual alignment scores for each reconstruction, and optionally iteratively comparing downstream alignments of extended reconstructions using subsequences available for reconstruction, and determining a reconstruction with the highest scoring alignment to thereby deduce the best alignment. The subunit sequences are preferably selected from nucleic acid sequences or amino acid sequences and more preferably from nucleic acid sequences.

In the practice of another embodiment of the present invention, the processing system executes a program for processing hybridization data. The program includes computer executable code, which when implemented on a suitable processing system, causes the processing system to receive data representing a set of overlapping subsequences, of length p, corresponding to a primary nucleic acid sequence, or to a complement thereof, comprising repeated subsequences of length p−1. The processing system is also adapted by the executable code to receive features of an oligonucleotide array whose probes detect specifically individual target oligonucleotide sequences under stringent hybridization conditions and to receive hybridization data from hybridization reactions between the oligonucleotide probes in the array and (1) the primary nucleic acid sequence and/or (2) at least one secondary nucleic acid sequence which varies from the primary nucleic acid sequence by the addition, deletion and/or substitution of at least one nucleotide, wherein the variation is associated with the alteration or destruction of at least one of the repeated subsequences. The processing system is also caused to process the hybridization data to determine which of said target sequences are contained in (a) the primary nucleic acid sequence and/or (b) the secondary nucleic acid sequence(s) to determine a set of subsequences, of length p, corresponding to the primary nucleic acid sequence and the secondary nucleic acid sequence(s), respectively. A comparison of overlapping subsequences, of length p, from both the primary nucleic acid sequence and secondary nucleic acid sequence sets is then executed by the processing system to wholly or partially determine the primary nucleic acid sequence.

The hybridization data may also be processed further by determining, for example, the signal intensity of the probes as a function of substrate position from the data collected, removing “outliers” (data deviating from a predetermined statistical distribution), and calculating the relative binding affinity of the target sequences from the remaining data. The resulting data can be displayed as an image with color in each region varying according to the light emission or binding affinity between target sequences and probes therein. In one embodiment, the amount of binding at each address is determined by examining the on-off rates of the hybridization. For example, the amount of binding at each address is determined at several time points after the nucleic acid sample is contacted with the array. The amount of total hybridization can be determined as a function of the kinetics of binding based on the amount of binding at each time point. Persons of skill in the art can easily determine the dependence of the hybridization rate on temperature, sample agitation, washing conditions (e.g., pH, solvent characteristics, temperature) in order to maximize conditions for hybridization rate and signal to noise.

The program for processing hybridization data may also comprise computer executable code which when implemented on the processing system causes the processing system to receive instructions from a programmer as input and/or to transform the data into a format for presentation.

The invention also features an algorithm comprising two main phases. The first phase uses the respective hybridization spectra of a primary nucleic acid sequence and at least one secondary nucleic acid sequence to construct subsequences. The second phase uses alignments between subsequences from the primary and secondary nucleic acids to infer the order of the subsequences.

In one embodiment of this type, the first phase of the algorithm considers each k-tuple in the hybridization spectrum, in turn, and extends it iteratively at both ends by adding overlapping k-tuples. The overlapping k-tuples must also be in the spectrum, and must overlap by exactly k-1 characters. The extensions at each end cease when there are no further overlapping k-tuples, or there is more than one possible overlapping k-tuple. A difficulty with this approach is that it will produce multiple copies of some subsequences, and some subsequences that are properly contained within one or more other subsequences. Accordingly, the algorithm preferably first identifies a subset of the k-tuples in the spectrum, called core edges, such that the subsequences produced by extending them in the manner described will be distinct and maximal, in the sense that they are not contained in any other subsequences. The core edges are found by an efficient graph-theoretical approach, which is known to persons of skill in the art.

For each maximal subsequence, the first phase of the algorithm also identifies all of the other maximal subsequences that could potentially overlap it. It does this efficiently, in time and space proportional to the total length of maximal subsequences.

Note that this first phase of the algorithm currently assumes perfect hybridization data—with no false positives or false negatives. Techniques for handling imperfect data are discussed infra.

The second phase of the algorithm orders the maximal subsequences of the primary and each secondary nucleic acid sequence in such a way that the reconstructed sequences have a convincing multiple sequence alignment. The procedure is as follows. In one embodiment, the first k-tuple of the primary and each secondary nucleic acid sequence must be input. The algorithm then aligns these k-tuples. The alignment is straightforward, since the algorithm assumes that the secondary nucleic acid sequences contain no insertions or deletions. Thus, the first base of the primary nucleic acid sequence is aligned to the first base of each variant, the second base of the target is aligned to the second base of each secondary nucleic acid sequence, and so on. The algorithm then finds all maximal subsequences of the primary nucleic acid sequence beginning with the specified k-tuple, and similarly for each variant. From this, the algorithm determines all possible ways to extend the multiple sequence alignment by exactly one base. That is, it finds all possible ways to extend the reconstructions of the primary nucleic acid sequence and each secondary nucleic acid sequence by exactly one base, and it generates all possible combinations of those possible extensions. The extended reconstructions are then re-aligned, and each multiple sequence alignment is scored in a manner to be described below. The best (lowest) scoring alignment is determined and alignments greater than some threshold value above this lowest score are discarded.

For each remaining alignment, the algorithm determines all possible ways to extend the alignment by one more base. This involves the following steps. Firstly, all possible ways to extend the reconstruction of the primary nucleic acid sequence are determined. If the reconstruction of the primary nucleic acid sequence ends in the middle of a maximal subsequence, then there will be only one possible extension, namely, the next character in the subsequence. However, if the reconstruction of the primary nucleic acid sequence ends at the end of a maximal subsequence, then the algorithm considers all possible overlapping maximal subsequences, as determined in the first phase, to determine the possible extensions. If there are no possible extensions, then the algorithm extends the primary nucleic acid sequence with a dummy character (for example ‘$’) to signify termination. All possible extensions of each secondary nucleic acid sequence are determined in a similar manner. The algorithm then forms every possible combination of the possible extensions and re-aligns them.

Each new alignment is then scored according to the scheme described below, the lowest score is determined and alignments with scores greater than the threshold value above this lowest score are discarded. The procedure described in this paragraph and the previous one is then iterated until the termination criteria are satisfied.

In one embodiment, the algorithm terminates when the alignments reach a pre-set length. This length does not have to be the exact length of the primary or original sequence, but it must be greater than or equal to that length. Other termination criteria are possible and may be used. Persons of skill in the art can determine such criteria without undue experimentation. The algorithm then considers the remaining alignments and outputs the one with the lowest score. If more than one alignment has the lowest score, then it outputs the one that appears highest in the list of alignments.

In another embodiment, the scores of the multiple sequence alignments are computed as follows. For each secondary nucleic acid sequence or variant, count the number of mismatches between the reconstruction of that variant and the reconstruction of the sequence it was derived from (which could be the primary nucleic acid or target sequence or another variant). The score of the alignment is the sum of these counts over all variants.

In order that the invention may be readily understood and put into practical effect, particular preferred embodiments will now be described by way of the following non-limiting examples.

### EXAMPLES

### Example 1

A New Paradigm for Experimental Data Collection, Processing and Analysis

As discussed above, the present invention provides a new paradigm for experimental data collection, processing and analysis, which is illustrated in FIG. 2. The new feature is the generation of modified copies (or variants) of the original objects. The original object(s) and the variant(s) are then subjected to various experimental procedures. Alternatively, experiments may be performed only on the variants as in FIG. 3. The resulting data is then processed to obtain information about the original object(s.

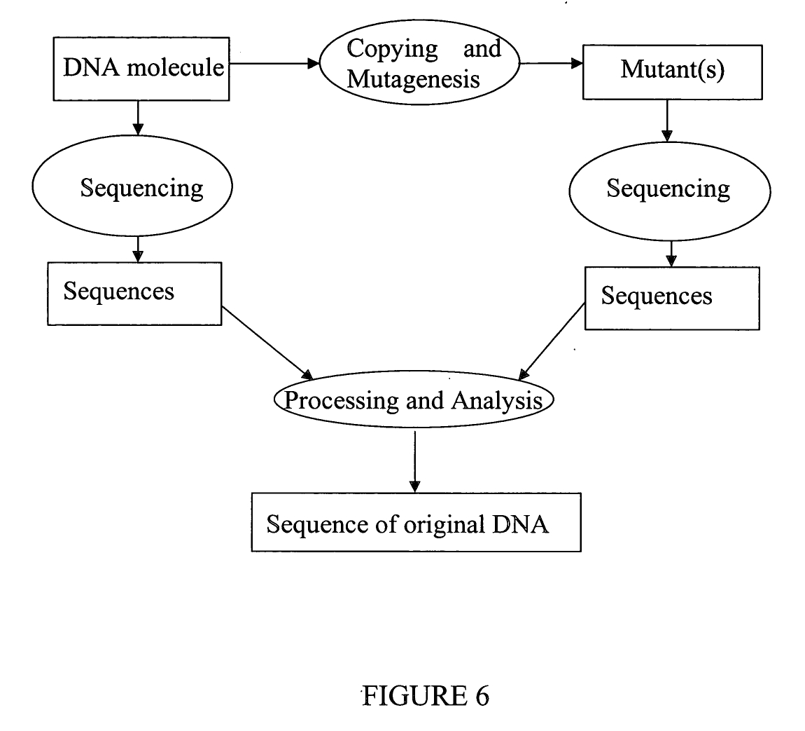

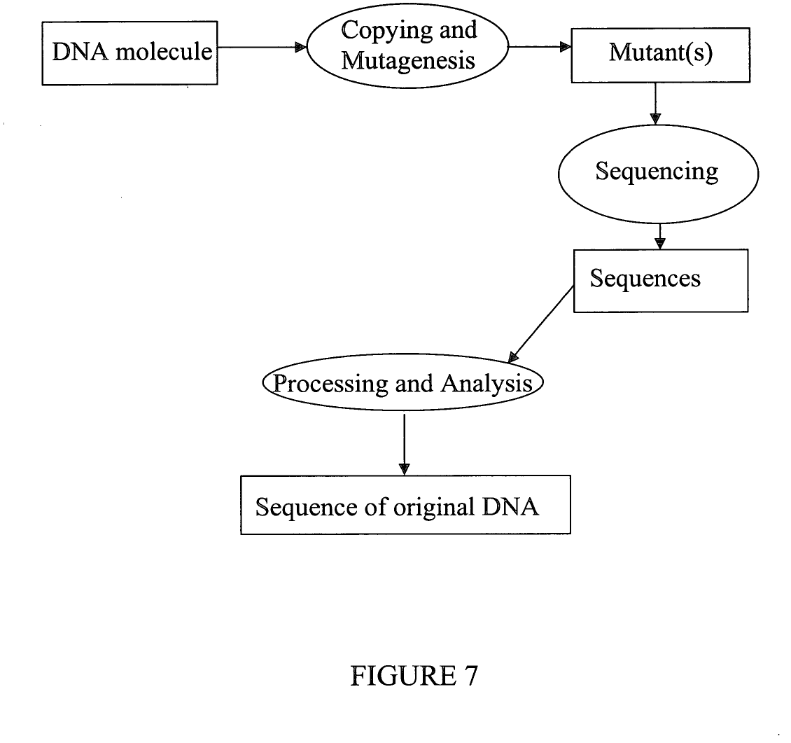

The paradigms represented in FIGS. 2 and 3 are particularized to sequencing applications in FIGS. 6 and 7, respectively. The original object is now a DNA molecule, and the information one aims to obtain is the primary sequence, in whole or in part, of that molecule. Modification is done by mutagenesis and the variants may therefore be described as mutants. The experiments involve sequencing of the original and/or mutant DNAs in whole or in part. The data generated by these experiments are sequences. It is to be understood that the sequences may contain some errors.

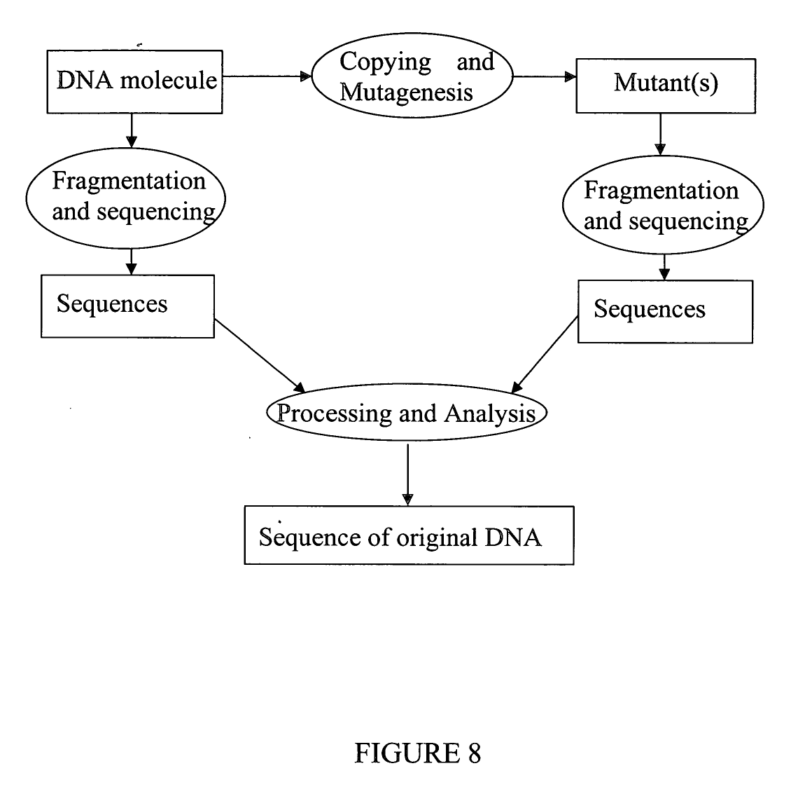

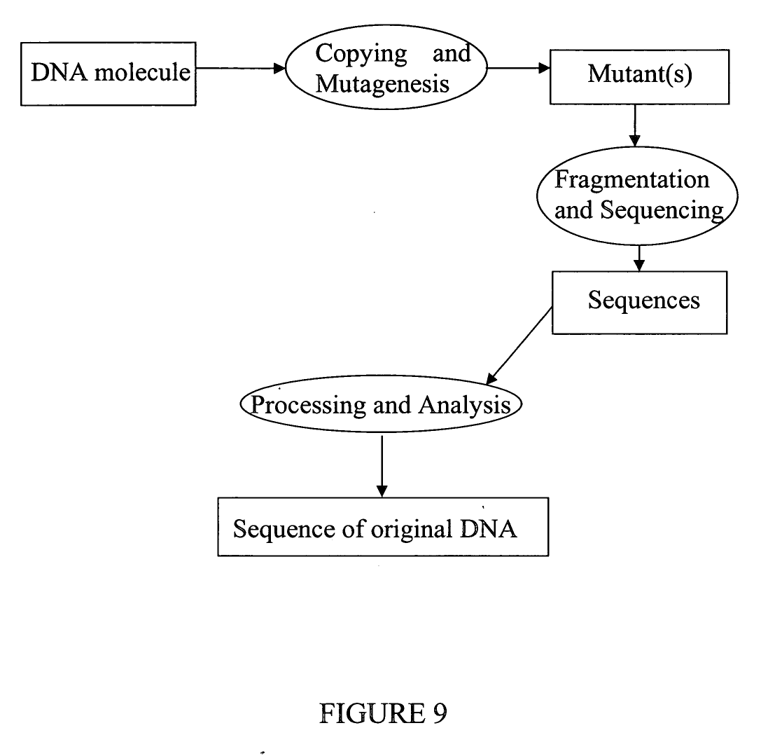

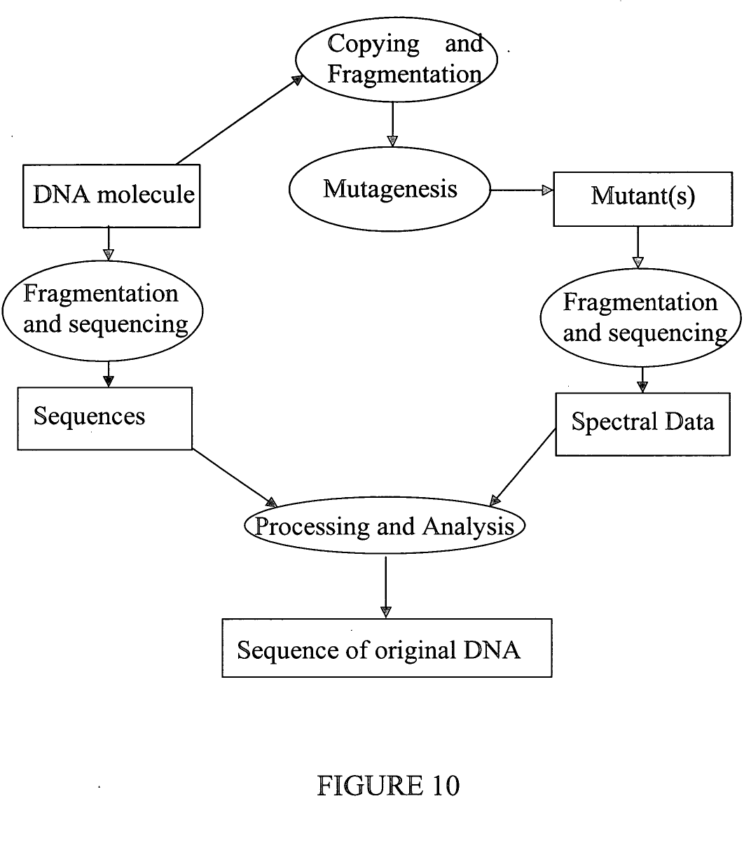

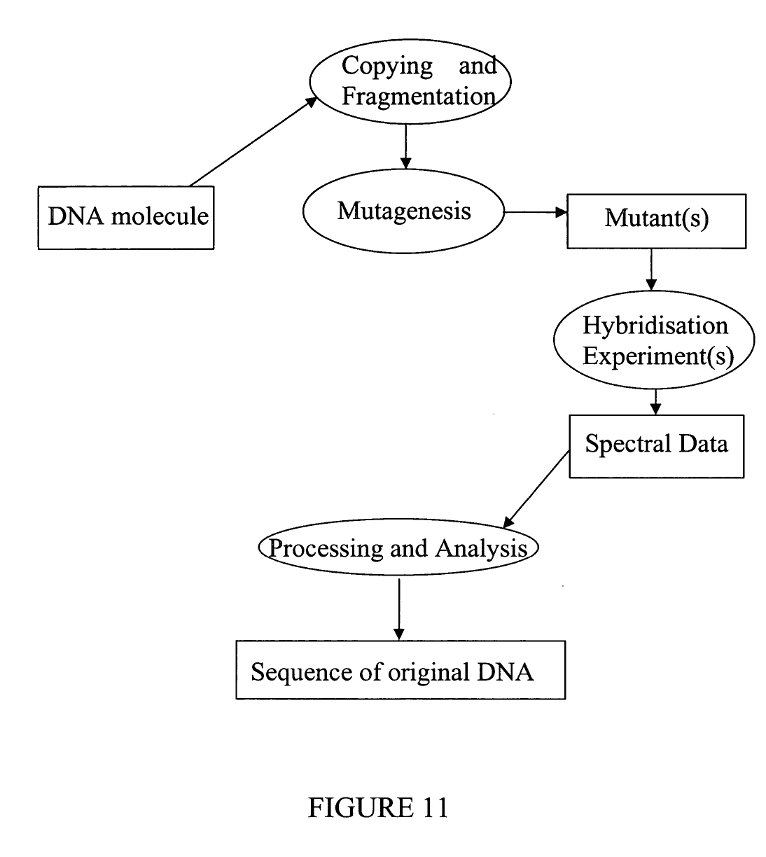

FIGS. 6 and 7 are further particularized to shotgun sequencing in FIGS. 8 and 9, respectively. In these diagrams, the word ‘Sequencing’ is replaced by the phrase ‘Fragmentation and sequencing’ in order to emphasize that the sequences obtained in shotgun sequencing represent only part of the original sequence. Two alternative approaches are illustrated in FIGS. 10 and 11. The difference in these latter two figures is that fragments of the original sequence are generated prior to mutagenesis. It is to be understood that FIGS. 10 and 11 are particularizations of FIGS. 6 and 7, respectively and of FIGS. 2 and 3, respectively. Consequently, the variants mentioned in FIGS. 2 and 3 may be variants of only a part of the original objects, and the mutants mentioned in FIGS. 6 and 7 may be mutants of only a part of the original DNAs.

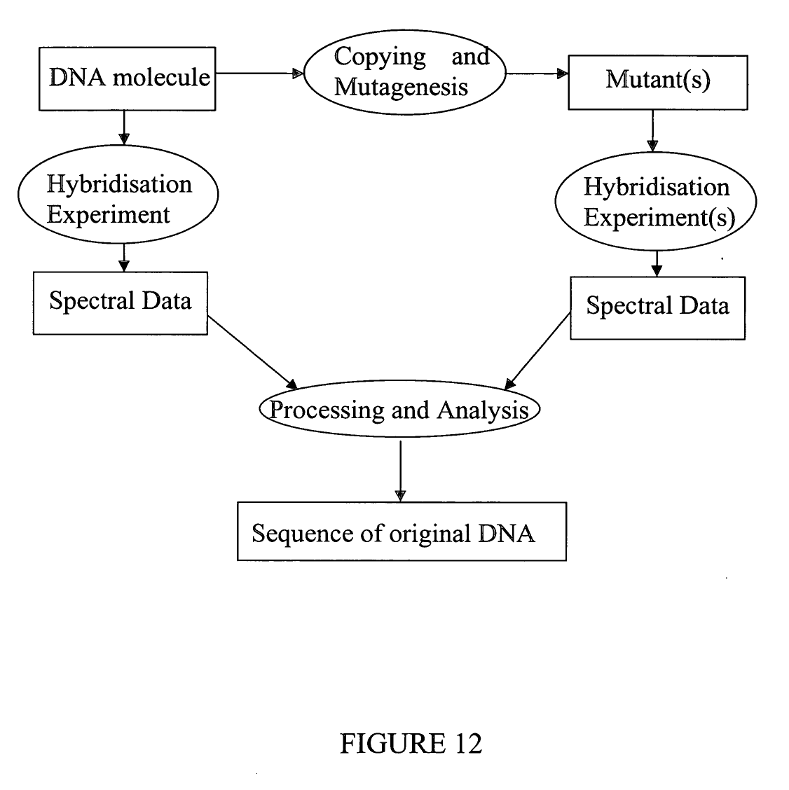

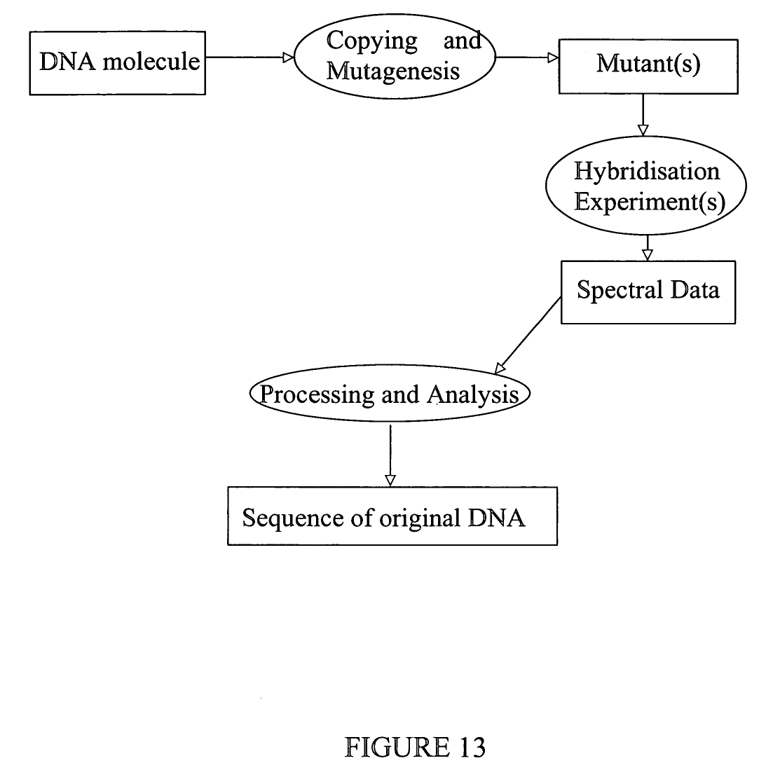

FIGS. 2 and 3 are particularized to SBH in FIGS. 12 and 13. As in FIGS. 6 and 7, the original object is a DNA and the desired information is the sequence of that molecule. Modification is done by mutagenesis and the variants are therefore mutants. However, the difference is that the experiments are hybridization experiments and the data are spectral data. The spectral data may comprise a list of pmers found to hybridize to the original sequence, or alternatively a measure of the strength of the hybridization signal for each probe. It is to be understood that spectral data may be imperfect. For example, they may contain false positives and negatives.

### Example 2

Simulated Mutation and Reconstruction

Mutation and SAM reconstructions were simulated for target sequences ranging in length from 0.5 kb to 30 kb and using probe lengths of ten and thirteen bases. Results for 5-star and 9-star mutation configurations (see Section 4) are shown in Tables 1 and 2, respectively. The third column of each table shows the percentage of fragments that the algorithm was able to reconstruct correctly, and the fourth column shows the percentage of fragments that were reconstructed with fewer than one in a thousand bases incorrectly identified. Approximately 1000 simulations were carried out for each row of the tables.

TABLE 1
Reconstruction efficiency of SBHM with
a 5-star mutation configuration.

| | | Percentage | Percentage |
|---|---|---|---|
| | Target | correctly | reconstructed |
| Probe length | sequence | reconstructed | to within 0.1% |
| (b) | length (kb) | using SBHM | using SBHM |
| 10 | 0.5 | 98.9 | — |
| 10 | 1 | 96.0 | 97.8 |
| 10 | 5 | 70.9 | 86.2 |
| 10 | 10 | 45.0 | 64.4 |
| 13 | 0.5 | 99.5 | — |
| 13 | 1 | 99.1 | 99.7 |
| 13 | 5 | 91.7 | 99.4 |
| 13 | 10 | 78.3 | 98.6 |
| 13 | 30 | 41.7 | 95.8 |

TABLE 2
Reconstruction efficiency of SBHM with
a 9-star mutation configuration.

| | | Percentage | Percentage |
|---|---|---|---|
| | Target | correctly | reconstructed |
| Probe length | sequence | reconstructed | to within 0.1% |
| (b) | length (kb) | using SBHM | using SBHM |
| 10 | 0.5 | 99.4 | — |
| 10 | 1 | 98.4 | 98.9 |
| 10 | 5 | 85.5 | 92.8 |
| 10 | 10 | 70.1 | 82.7 |
| 13 | 0.5 | 99.9 | — |
| 13 | 1 | 99.9 | 100.0 |
| 13 | 5 | 97.8 | 99.9 |
| 13 | 10 | — | — |
| 13 | 30 | — | — |

In the present example, the inventors have attempted to provide a conservative lower bound on the potential effectiveness of SAM. Although the above simulations might not account for all the laboratory issues associated with the method, it is believed that the major limiting factor on reconstruction efficiency is the pattern of repeats in the target sequences. Previously sequenced human DNA has, therefore, been used to ensure that the pattern of repeats is realistic.

The results demonstrate a dramatic improvement over the reconstruction efficiency of standard SBH. For example, in a recent unpublished study the inventors estimated that the probability of unambiguously reconstructing a 1 kb fragment of human DNA using standard SBH and probes of length ten is only 2%, whereas the probability of correctly reconstructing such a fragment using SAM and a 9-star mutation configuration is at least 98%. It should be mentioned that these percentages cannot be directly compared because a correct reconstruction does not imply an unambiguous reconstruction. The SAM algorithm described herein may occasionally select the correct reconstruction even when there are equally likely alternatives. Nevertheless, the improvement is clear.

For long fragments (5 kb or more), there is also a substantial improvement when a 9-star mutation configuration is used instead of a 5-star mutation configuration. This validates one of the key concepts of SAM: that each mutation resolves a different set of reconstruction ambiguities.

The fourth column of the Tables 1 and 2 demonstrates another attractive feature of SAM. If one error per thousand bases is allowed, then the reconstruction efficiency is significantly higher. This indicates that many of the unresolved ambiguities involve only a small number of bases. In standard SBH, a reconstruction ambiguity generally means that two or more very different reconstructions are possible. However, it appears that SAM can typically achieve a correct overall order of maximal subsequences even when a small number of short subsequences are misplaced.

### Example 3

Re-Construction of Original DNA Sequence From a Series of Mutated Copies

An unspecified target DNA sequence (˜1.5 Kb) cloned into a sequencing vector (pUC 19) was used as the template for in vitro mutagenesis. Mutagenesis was achieved using PCR in the presence of nucleotide analogues: 6-(2-deoxy-Beta-D-ribofuranosyl)-3,4dihydro-8H-pyrimido-[4,5 c][1,2]oxazin-7-one 5′-triphosphate (also known as dPTP), 8-oxo-2′deoxyguanosine 5′-triphosphate, triethylammonium salt (also known as 8-oxoGTP), or both together. The partial sequence of the unmutated clone is:

| | |
|---|---|
| GGCGTAATAATACTATTTGTTGTGTCAATTTTCTTGGTTCCTGACTAAAACATTAAGGTTTC | [SEQ ID NO:157] |
| TCAGTTAAGCTATATACGATAAATATTGGCATCTTTCTATTGCAGGATGATTTCTAGTGCTA | |
| AGCATTATAGCCAGGAGTAAAGGAAATAACGCTTTAACGATACCACCATTAATTTAAAAAAT | |
| GGAGTCTGAAATGGAAAAAGAAGAAAAAAGCAATCTCATCTACGATAAAGATCCTGGATATG | |
| TGT | |

The mutation method is derived from that published by Zaccolo, M., et al. (1996, J. Mol. Biol. 255: 589-603). Universal M13 primers (FSP-21, FSP-40, RSP-26, RSP-48) were used for PCR amplification of purified and unmutated plasmid DNA, and for sequencing. Standard PCR reaction conditions were used with nucleotides at a concentration of 400 μM for dATP, dCTP, dGTP, dTTP. The reaction was supplemented with either 400 μM dPTP or 400 μM 8-oxo-GTP or both. In this type of PCR nucleotide analogues were incorporated in place of the natural nucleotides at a relatively low frequency per cycle but progressively accumulating with increasing numbers of cycles. The products of this PCR were re-amplified in the absence of the analogues to exchange the incorporated analogues with natural nucleotides thereby creating and fixing the sequence changes.

Twenty mutant sequences were generated using dPTP only, 4 mutants were generated using 8-oxo-GTP only and two mutants were generated using both nucleotide analogues. The dPTP mutants were found to differ from the original sequence in approximately 20% of bases on average. The 8-oxo-GTP mutants were found to differ from the original sequence in approximately 3% of bases on average. The mutants generated using both nucleotide analogues were found to differ from the original sequence in approximately 4% of bases on average.

Reaction Conditions:

2 ng DNA template, 1× AmpliTaq Gold buffer, 400 μM dNTPs, 2 mM magnesium chloride, 0.4 μM each primer, 1 unit of AmpliTaq Gold, in a total of 25 [symbol] L. Reactions were performed as follows: 1 cycle of 94° C. for 15 min., 30 cycles of 94° C. 1 min, 50° C., 0.5 min, 72° C. 5 min., 1 cycle 72° C. 10 min. This yields PCR products incorporating analogue bases. Viable mutated DNAs were recovered by re-amplification of 1 μL of the reaction products with nested primers and the four natural dNTPs.

Cloning of PCR Products and Sequencing:

The mutant PCR products were gel purified and cloned into the pGEM T-EASY vector (Promega) and transformed into E. coli. Plasmid DNA from individual clones were sequenced and analyzed for mutation frequency.

Reconstruction

The data were separated into two groups: the 20 dPTP mutants in one set (see Example 5) and the remaining 6 mutants in the other set (see Example 4). Each group was used to independently reconstruct the original sequence. For each set, the well-known multiple sequence alignment package ClustalW was used to align the sequences. A consensus sequence was then obtained for each set by finding the most frequent character in that column. Where there was no most frequent character, an ‘N’ was placed. The consensus sequences were determined using C code that we wrote for the purpose.

Algorithm

The reconstruction algorithm used in this example and in Examples 4 and 5 is used to infer the sequence of a short DNA fragment given the sequences of a number of mutants. The algorithm consists of the following steps.

a) Align the mutant sequences using ClustalW.

b) Determine the most frequent character in each column of the alignment.

c) Concatenate these characters to form a consensus sequence.

d) Output consensus sequence.

### Example 4

Reconstruction 1 of Sequences Generated in Example 3

The alignment of the 6 mutant set is shown below. Also shown in this alignment are the consensus sequence and the original sequence. Probable mutations are shown in lower case. Observe that the consensus sequence is identical to the original sequence. In other words, the original sequence has been successfully reconstructed without errors.

| | |
|---|---|
| 8_OXO_1F-edit | GGCGTAATAATACTATTTGTTGTGTCAATTgTCTTGGTTCCTGACTAAAACATTAAGGTT |
| 8_OXO_2F-edit | GGCGTAATAATACTATTTGTTGTGTCAATTTgCTgGGTTCCTGACTAAcACATTcAGGTT |
| 8_OXO_3F-edit | GGCGTAATAATACTATTTGTTGTGTCAATTTTCTTGGTTCCTGACTAAAACATTAAGGTT |
| 8_OXO_4F-edit | GGCGTAATAATACTATgTGTTGnGTCAATTTTCTTGGTTCCTGACTAAAACATTAAGGTT |
| BOTH_1F-edit | GGCGTAATAATACTATTTGTTGTGTCAATcTcCTcGGTTCCTGACTAAAACATTAAGGTT |
| BOTH_2F-edit | GGCGTAATAATACTATccGTTGTGTCAATTTTCTTGGTTCCTGACTAAAACATTAAGGTT |
| consensus | GGCGTAATAATACTATTTGTTGTGTCAATTTTCTTGGTTCCTGACTAAAACATTAAGGTT |
| original | GGCGTAATAATACTATTTGTTGTGTCAATTTTCTTGGTTCCTGACTAAAACATTAAGGTT |
| 8_OXO_1F-edit | TCTCAGTTAAGCTATAgACGATAAATATTGGCATCTTTCTATTGCAGGATGATTTCTAGT |
| 8_OXO_2F-edit | TCTCAGTTAAGCTATATACGATAcATATgGGCATCTTTCTATTGCAGGATGATTTCTAGT |
| 8_OXO_3F-edit | TCTCAGTTAAGCTATATACGATAAATCTTGGCATCTTgCTATTGCAGGATGATTTCTAGT |
| 8_OXO_4F-edit | TCTCAGTTAAGCTATnTACGATAAATATTGGCATCTTTnTATTGCAGGATGATTTCTAGT |
| BOTH_1F-edit | TCTCAGTTAAGCTATAcACGATAAAcAcTGGCATCcTTCTAcTGCAGGATGATcTCcAGT |
| BOTH_2F-edit | TCTtAGTTAAGCTATATACGATAAATATTGGCAcCTTTCTATTGCAGGATGAcTTCTAGT |
| consensus | TCTCAGTTAAGCTATATACGATAAATATTGGCATCTTTCTATTGCAGGATGATTTCTAGT |
| original | TCTCAGTTAAGCTATATACGATAAATATTGGCATCTTTCTATTGCAGGATGATTTCTAGT |
| 8_OXO_1F-edit | GCTAAGCATTATAGCCAGGAGTAAAGGAAATAACGCgTTAACGATACCACCATTAATTTA |
| 8_OXO_2 F-edit | GCTAcGCATgATAGCCAGGAGTAAAGGAAATAACGCTTTAACGcTACCACCATTAATTTA |
| 8_OXO_3 F-edit | GCTAAGCAgTATAGCCAGGAGTAAAGGAAATcACGCTTTAACGATACCACCATTAATTTA |
| 8_OXO_4 F-edit | GCTAAGCATTATAGCCAGGAGTAAAGGAAATAACGCTTTAACGATACCACCATTAATTTA |
| BOTH_1F-edit | GCTAAGCATTATAGCCAGGAGTAAAGGAAATAACGCTTTAACGATACCACCATTAATcTA |
| BOTH_2F-edit | GCTAAGCATTATAGtCAGGAGTAAAGGAAATAACGCTTTAACGATACCACCATTAATTcA |
| consensus | GCTAAGCATTATAGCCAGGAGTAAAGGAAATAACGCTTTAACGATACCACCATTAATTTA |
| original | GCTAAGCATTATAGCCAGGAGTAAAGGAAATAACGCTTTAACGATACCACCATTAATTTA |
| 8_OXO_1F-edit | AAAAATGGAGTCTGAAATGGAAAAAGAAGAAAAAAGCAATCTCATCTACGATAAAGATCC |
| 8 OXO 2F-edit | AAAAATGGAGTCTGAAAgGGAAAAAGAAGAAAAAcGCnATCTCATCTACGATAAAGATCC |
| 8_OXO_3F-edit | AAAAATGGAGTCTGAAcTGGAAAAcGAAGAAAcAAGCAATCTCATCTACGATAAAGATCC |
| 8_OXO_4F-edit | AAAAATGGAGTCTGnAcTGGAAAAAGAAGAAAAAAGCAATCTCATCTACGATAAAGATCC |
| BOTH_1F-edit | AAAAATGGAGcCTGgAATGGAAAAAGAAGAAAAAAGCAATCTCATCTACGATAAAGATtC |
| BOTH_2F-edit | AAAAATGGAGTCTGAAATGGAAAAAGAAGAAAAAAGCAAcCTCATCTACGATAAAGATCC |
| consensus | AAAAATGGAGTCTGAAATGGAAAAAGAAGAAAAAAGCAATCTCATCTACGATAAAGATCC |
| original | AAAAATGGAGTCTGAAATGGAAAAAGAAGAAAAAAGCAATCTCATCTACGATAAAGATCC |
| 8_OXO_1F-edit | TGGATAT [SEQ ID NO:158] |
| 8_OXO_2F-edit | TGGATAT [SEQ ID NO:159] |
| 8_OXO_3F-edit | gGGATcT [SEQ ID NO:160] |
| 8_OXO_4F-edit | TGGATAT [SEQ ID NO:161] |
| BOTH_1F-edit | TGGATAc [SEQ ID NO:162] |
| BOTH_2F-edit | TGGATAT [SEQ ID NO:163] |
| consensus | TGGATAT [SEQ ID NO:164] |
| original | TGGATAT [SEQ ID NO:165] |

In this case, it was possible to correctly reconstruct the sequence using only a small number of mutants.

### Example 5

Reconstruction 2 of Sequences Generated in Example 3

The alignment of the 20 dPTP mutants is shown below, together with the consensus sequence and the original sequence. Mutations are shown in lower case. Observe that the consensus sequence is identical to the original sequence. The consensus character in each column was not obtained by finding the majority character as in Example 4. Instead, the following criteria were used. If a column contained more than 7 A's, the consensus character was taken to be A. Otherwise, if a column contained more than 13 C's, the consensus character was taken to be a C. Otherwise, if a column contained more than 12 G's, the consensus character was taken to be a G. Otherwise, if a column contained more than 6 T's, the consensus character was taken to be a T. Otherwise, if a column contained a simple majority of dashes, the consensus character was taken to be a dash. Otherwise, the consensus character was undetermined. These criteria can be justified using Bayesian probability and the information that the probability of replacing any given A with a G is approximately 0.23, the probability of replacing any given G with an A is approximately 0.08, the probability of replacing any given T with a C is approximately 0.23 and the probability of replacing any given C with a T is approximately 0.05. There were no undetermined characters in this example.

| | |
|---|---|
| seq_1 | GGCGTAATAAcACTATTTGTCGTGcCAATTTTCTTGGTTCCTGgCTAAAgCATTAgGGTc |
| seq_2 | GaCGTAgTAgTACTATcTGTcGTGTCAgTcTTCcTGGTTCCcGACcAAgACAcTAAGGTc |
| seq_3 | GaCGTAgTAgTACTATTTGCTGTGcCAAccTTCTTaGTcCCTGgCTgAAgCATTgAGGTT |
| seq_4 | GGCGTgATAAcgCTAccTGcTacacCAATccTCcTGGcTCCTGgCcAAggCAcTAAGGTc |
| FTP_1F | GGCGTAATAATACcAcTTGTTGTGTCAATTTcCTTGGTTCCTGACTAAAACAcTAAGGTc |
| ptpfor001 | GGCGTAATgATACTAccTGTcGTGcCAATcTTCTTGGTTCCTGACTAgAgCATTAAGGTT |
| ptpfor002 | GGCGTAgTAATACcATcTacTGTGTCAATcccCTcGacTCCcGACTgAAACATTAAGGTT |
| ptpfor003 | GGCGTAAcAgTACTAcTTGcTGcGTCAAcTTTCTTGGTcCCTGgCTgAAgCgTTAAGGcc |
| ptpfor004 | GGCGTAATAATACTATcTGTTGTGTCAATTTTCTTGGTTCCTGACTAAAgCATTAAGGTT |
| ptpfor006 | GGCGTAATAATACTAccTGTTGcGcCAATcTcCcTGGTcCCTGACTAAAACgTTAAGGTc |
| ptpfor007 | GGCGTAATAATACTAcTTacTGTGTtAATTcTCTcGGTcCCcGACTAAAACATTAAGGTT |
| ptpfor008 | GGCGTAATAATACTgTTTGTTGTGTCAATTTTCTTGGTTCCTGACcAAAACATTAAaGTT |
| ptpfor009 | aaCGTAATAATACTATTTGccGTGTCAATTTTCTcGGTcCCcGACTgAAAtgTTAgGGTT |
| ptpfor010 | GGCacAATAATAtTATTTGTcGnGTCAgTcTctCTGGcTCCTGACcAAAACgTcAAGGTc |
| ptp4 | GGCGTAAcAgcACcAcTTGTTGTGcCgATTccCTTGGTcCCcGACTAAgACAcTAgGGTc |
| ptp18 | GGCGTAATAgTACTATTTGTTGTGTCAATTcTCTTGGcTCCTGACTgAAACAcTAAGGTc |
| ptp20 | aaCGTAATAgTACcATTcGTTGTGTCAAcTTTCTTGGccCCTGACTAgAgCATcgAGGTc |
| ptp21 | GGCGTAgcggTACTgTTcGcTGTGTCgATcTTCTTGGccCCTGACTAgAgCATTAAGGTC |
| ptp22 | GGCGTAgTAATACTAcTTGTTGTGTCAAcTTTCTTGGTcCCTGACTAAAACATTAAGGTT |
| ptp23 | --------------------------------------------------CATTAAGGTc |
| consensus | GGCGTAATAATACTATTTGTTGTGTCAATTTTCTTGGTTCCTGACTAAAACATTAAGGTT |
| original | GGCGTAATAATACTATTTGTTGTGTCAATTTTCTTGGTTCCTGACTAAAACATTAAGGTT |
| seq_1 | TCTCgGTTAgGCTgTATACGgcgAgTgTTGGCATCTTcCTATcGCgGGATGATTTCTAGT |
| seq_2 | TCTCAGcTAgaCTgTgcACGATAAATATTGGCgcCTTcCTAcTGCgGaATGAcTTCTAaT |
| seq_3 | TCcCgGTcAAaCcATAcgCGATAAgTATTGGCAcCTTTCTAcTaCAGGATGgcTTCTAGT |
| seq_4 | TCTCAGTcggGCTATATACagTAggcATTGGCATtccTtTgTcGtgGGgTaATcTCTAGT |
| PTP_1F | TtTCAGcTAAGCcgTATACGATAAAcAcTGGCATCTTTCTgcTaCAGGgcGATTcCTAGT |
| ptpfor001 | TCTCAGTTAAGCTATATACGgTAAATATTGGCATCTTcCTATTGCAGGATGATTTCTAGT |
| ptpforo02 | TCTCAGcTAAGCTAcgcACGATgAgTAccGGCATCccTCTATcGCAGGAcGATccCTAGT |
| ptpforO03 | TCTCgGTTgAGCTATgcACGgTAAATAcTGaCgctTccCcgTcGCAGGgcGATcTnTgGc |
| ptpforo04 | TCTCAGTTAAGCTgTATACGATAAATATTGGCAcCTTTCTATTGCAGGATGATTTCTgGT |
| ptpfor006 | TCcCAGTTAAGCcAcATACGAcAgATATTGGCATCcccCTAcTGCgGaATGATTTCTgGT |
| ptpfor007 | TCcCAGTTAAGCTATATAtGgTAAATgccGGCAcCTTTCTATcGCAGGATGATcTCTAGc |
| ptpfor008 | TCTCAGTTAAaCTATAcACGgcAgATATTGGtAcCTTcCTATTGCAGGgTGATTTCTAGc |
| ptpfor009 | TCcCAGTTAAGCTATATACGATAAATATTGGCATCTTTCcATTGCgGGATGATTcCTAGT |
| ptpfor010 | cCcCAGTcAAGCcAcATACGAcAgAcATTGGCATCTTcCTAcTaCAGGATGATTTCTAGT |
| ptp4 | cCcCAGTcAgaCcATATACGATAAATAccGGCAcCTcctTAccGCgGGAcagTTcCTAac |
| ptp18 | TCTCAGcTAgGCTATgTgCGAcggATATTGGCATCcTTCTgcTaCAGGATGAcTTCTAGc |
| ptp20 | TCTtgGTTAAGCTgcATgCGATAAATATTGGCgcCTcTCTAcTGCAGGgTGgTccCTgGn |
| ptp21 | TCTtgaTTAAGCTATgcACGgTgAATAcTGGCATCcTcCTATcGCAGGAcGgccTCTgGT |
| ptp22 | TCcCAGcTAAGtcATATACGATAAATAcTGGCgTtcTTCTAcTGCgGGAcGgcTcCTgGT |
| ptp23 | cCTCgGTTgAGCTATgTACGgTgAgTATTGGCgcCTTcCTATTGCAGaATaATTTCTAGc |
| consensus | TCTCAGTTAAGCTATATACGATAAATATTGGCATCTTTCTATTGCAGGATGATTTCTAGT |
| original | TCTCAGTTAAGCTATATACGATAAATATTGGCATCTTTCTATTGCAGGATGATTTCTAGT |
| seq_1 | GCTAgaCgcTATAGCCAGGgGTAAAGGAAgTAACGCTTcAgCGgTACCACCATTAgTTTA |
| seq_2 | GCTAAGCAcTATgaCCAGGAGTggAGGAggcAACaCTcTAACGAcACCACCATTAATTcA |
| seq_3 | GCcAgaCATTAcAGCCAGGgGTgAAGGggATAACGCTTTAgCGAcACCACCgTTAAccTA |
| seq_4 | GCTAAaCATTATAGCCAGGgGTgAAGGAAATAACGCTcTAACGATACCACCgcTAgcccA |
| PTP_1F | GCTAgGCATTATqGCCAGGAGTAAAGGggATgACGCTTcAgCGgcACCgCCATTggTTTA |
| ptpfor001 | GCcgAGCATTATAGCCAGGAGTAAAGGAAATggCGCcTTAgCGgTgCCACCATTAgTTTA |
| ptpfor002 | GCTAgGCATcAcAGCCgGGAGTAAgGGAgATAACaCTTTAACGgTACCACCAcTAAcTcg |
| ptpfor003 | GCcAAGCATnATAGCCAGGgGTAAAGGAAgTAACGCTTTggCGgcACCACCAcTAAcTTA |
| ptpfor004 | GCTAAGCATTAcAGCCAGGAGTAAAGGAAATAACGCTTTAACGgTACCACCATTAATTTA |
| ptpfor006 | GCTAAGCgTTATAGCCAGGAGTAAAGGgAATgACGCcTTAACGgTACCgCCgTTAATTcA |
| ptpfor007 | GCcAgGCgcTATAGtCAGGAGTAgAGGAgATgACGCTTTAACGATACCgCCATcAAcTTg |
| ptpfor008 | GCTAAGCATcATAGtCAGGAGTAAAGGggATAACGCTTTggCGATACCACCATTAATcTA |
| ptpfor009 | GCTAAGCATTATAGCCAGGAGTAAAGGAAATAACGCTTTAgCagTACCACCATTAATTTA |
| ptpfor010 | GCTAAGtgTTgTAGCCAGGAGcAAAGGAgATAACGCccTAAtGgcgCCAtCATTAATTcA |
| ptp4 | GCTAAGCAcTATAGCCgGGgGTAAAGGggATAACGCTTcgACGATACCACCgcTAAcTTA |
| ptp18 | GCTggGCgccATAGCCAGGAGTAAAGGggATAAtGCTcTAACGgCACCACCAcTAATTTA |
| ptp20 | GCTggGCgTTATAGCCgGGAGTAAgGGAgAcAACGCTTnggCGgcACCACCAcTAgTTTA |
| ptp21 | GCTgAGCgcTATgGCCAGGAGTgAAGGggATgACGCcTTAACagTACCACCgTTAgTTcA |
| ptp22 | GCTAAGCgTTATAGCCAGGAGTggAGGAAATAACGCTTTAgCGATACCACCATTAATTTA |
| ptp23 | GCcAAGCATTgTAGCCgGGgGTAAAGGAAAcAgCGCTTcAACGATACCgCCAcTAAcTcA |
| consensus | GCTAAGCATTATAGCCAGGAGTAAAGGAAATAACGCTTTAACGATACCACCATTAATTTA |
| original | GCTAAGCATTATAGCCAGGAGTAAAGGAAATAACGCTTTAACGATACCACCATTAATTTA |
| seq_1 | AAgggTGGgGcCTGAAgTGGAAAAAGggaAAgAAAGCAATCTCATtTcgGnnAAga---t |
| seq_2 | AAgAgcGGAGcCTGggATGGggAgAGggGAAAAgAGCAAcCcCATCTACaATAAAaganC |
| seq_3 | AAAgATGGAGTCTGAAATGGAAAAAGggGAgAgAAGCAATCTCgcCTACGAcAAAaA-cC |
| seq_4 | AAAAgTGGAGTCcGggATGGAgAAAGAgGAgAggAGCAATCcCg-CTgCanTAAAGgccC |
| FTP_1F | AAgAATGGgGcCTGAAATGGAgAAAGAgGgAAgAgGCAATCTCATnTgCGATAgAag-tC |
| ptpfor001 | AAgAATGGAGTCTGAAATGGAAAAAGAAGgAAAAAGCAgcCTtATCTACGATAAgGA-tC |
| ptpfor002 | gAgAATGGAGTtTGAAgTGGAAAAgGggGAAAAAAaCAATCTCgTCTgCGgTAgAGg-tC |
| ptpfor003 | gAgAATGGAGTCcGggATGGgggAAGggGAAAgAgaCgAcCcCAcCTACGgTgggGA-cC |
| ptpfor004 | AAAAATGGAaTnTGAAATGGAAAAAGAAGAgAAAAGCAATCTnATCTACGgTnAAGA-cC |
| ptpfor006 | AAgAgTGGAGTCTGgAgTGagAAAAGgAGAAggAAGCAgTCcCATCTgCaATAAgGg-tt |
| ptpfor007 | AgAAgTGGAGTCTGAAAcGGAggAgGAAGgAAAAAatAATCTCATtcACGATAgAaA-cC |
| ptpfor008 | AAAAgTGGAGTCTGAAAcGGAAgAAGAgGAAgAgAGtAATCTCATCTACGgcAAAGg-tC |
| ptpfor009 | AAAgATGGAGTCTGAAgTGGAAAAgGAgGAAAAAAGCAATCTCATCTACGATAAAGA-tC |
| ptpfor010 | gAAAgTGGAGcCTaAAgTG-gAgAAagAGAAgAAgagtAcCcCgTCTACagTnAAGA-cC |
| ptp4 | AgggATGGgGcCTGAgATGGAgAgAGAAGAAgAgAGCgATCTCATCTACGATAAgGg-cC |
| ptp18 | AgAAATGGAGTCTaAAgTGGgAAAAGAAGAAAAgAGCAATCTCAcCcACGAcgAgGA-tC |
| ptp20 | AggAATGGAGTCTGAAAcGGAAggAGAAGAgAAAgGCAATCcCATCTACaATAAAGA-tC |
| ptp21 | gAAAgcGGAGTCTGAgATGGAggAgGAAGgAAgAAGCAATCTtATCTACGgTAAAGA-tC |
| ptp22 | AAAAATGGAGTCTGAgATGGgAgAgGAAGAAgAAAaCAgTCTCAcCTACGAcAAgGA-cC |
| ptp23 | AAgAATGGAGTCTGgAgTGGAgAAAaggG~AgAgAGCAgTCcCATCcACGgTAAAGA-tC |
| consensus | AAAAATGGAGTCTGAAATGGAAAAAGAAGAAAAAAGCAATCTCATCTACGATAAAGA-TC |
| original | AAAAATGGAGTCTGAAATGGAAAAAGAAGAAAAAAGCAATCTCATCTACGATAAAGA-TC |
| seq_1 | CcnGgTtT [SEQ ID NO:166] |
| seq_2 | CTtGATtT [SEQ ID NO:167] |
| seq_3 | CTtGATnc [SEQ ID NO:168] |
| seq_4 | CTGGAcAT [SEQ ID NO:169] |
| FTP_1F | CTGGATAT [SEQ ID NO:170] |
| ptpfor001 | CTGagTAT [SEQ ID NO:171] |
| ptpfor002 | CcGGgcgT [SEQ ID NO:172] |
| ptpfor003 | CcGGgTAT [SEQ ID NO:173] |
| ptpfor004 | CTGGgTAT [SEQ ID NO:174] |
| ptpfor006 | CcGGgTAT [SEQ ID NO:175] |
| ptpfor007 | CTGGATAT [SEQ ID NO:176] |
| ptpfor008 | CTGGgTAT [SEQ ID NO:177] |
| ptpfor009 | CTGGATAc [SEQ ID NO:178] |
| ptpfor010 | CcGGgTAT [SEQ ID NO:179] |
| ptp4 | CTGGATAT [SEQ ID NO:180] |
| ptp18 | CTGGATAT [SEQ ID NO:181] |
| ptp20 | CTGGgTAT [SEQ ID NO:182] |
| ptp21 | CTGGATAT [SEQ ID NO:183] |
| ptp22 | CTGGATAT [SEQ ID NO:184] |
| ptp23 | CTGGATAT [SEQ ID NO:185] |
| consensus | CTGGATAT [SEQ ID NO:186] |
| original | CTGGATAT [SEQ ID NO:187] |

Algorithm

For this example, the inventors employed a modified version of the algorithm described in Example 3, which consists of the following steps.

a) Align the mutant sequences using ClustalW.

b) Determine the most probable original character for each column of the alignment, using Bayesian probability and estimated probabilities of the various substitutions.

c) Concatenate these characters to form a consensus sequence.

d) Output consensus sequence.

Persons of skill in the art will recognize that code for this algorithm can be easily modified to use other suitable multiple alignment software instead of ClustalW, such as TCOFFEE.

### Example 6

SAM Applied to Shotgun Sequencing I

Shotgun sequencing is a method for determining the primary sequence of a long DNA molecule (>10 kb). The method includes the following key steps:

1. Fragmentation of the DNA into smaller pieces.

2. Amplifying these fragments by cloning and/or PCR

3. Sequencing the fragments from one or both ends to determine short (300-1000 bp) sequences.

4. Piecing the sequences together to reconstruct the primary sequence of the original DNA molecule, or parts thereof.

Note that the reconstruction process must account for sequencing errors. Advanced reconstruction algorithms make use of ‘mate-pair’ information, that is, the approximate distance between the sequenced ends of the fragments. The total length of sequence obtained is usually enough to cover the original fragment 5 to 10 times.

Many genomes, including the human genome, contain many copies or near copies of certain sub-sequences. This causes significant problems in the reconstruction phase of shotgun sequencing. The problem is that for a given set of fragments, there may be more than one plausible reconstruction.

The SAM technique may facilitate various aspects of shotgun sequencing. Firstly, parts of the original DNA molecule that are refractory to the method of cloning or sequencing may be rendered amenable to sequencing via mutation. Secondly, mutation renders the DNA molecule less repetitive, and thus easier to reconstruct.

To demonstrate that introduced mutations facilitate reconstruction, the inventors performed computer simulations of mutation and shotgun sequencing as follows. A 120 kb sequence of human genomic DNA was obtained from GenBank (Accession Number AC000003). Short (300 bp) fragments of the original sequence were selected at random, the total length of the selected fragments being sufficient to cover the original sequence 1, 3, 5, 7 and 9 times (in different simulations). Random substitutions were introduced into the reads to simulate sequencing errors (NOT mutations). The probability of any given base being modified was 0.03. The shotgun reconstruction program ‘phrap’ was then used to attempt reconstruction of the original sequence. The inventors recorded the number of times phrap failed to recognize that two fragments should be adjacent to each other and the number of times it incorrectly placed two reads adjacent to each other in its reconstruction. This was repeated 100 times for various amounts of coverage and the results averaged (see Table 3—0% substitution).

This entire process was then repeated, but using simulated mutants instead of the original sequence. The mutants were generated by considering each character in turn and making a substitution with probability 0.05, 0.1, 0.15, 0.2, 0.25 and 0.3 (in different simulations). These results are also shown in Table 3.

TABLE 3
Performance of phrap on mutated sequences

| | | # joins | # incorrect | |
|---|---|---|---|---|
| Coverage | % substitution | missed | joins | Time(s) |
| 1 | 0 | 36.6 | 87.1 | 7.1 |
| 1 | 5 | 34.9 | 39.0 | 5.0 |
| 1 | 10 | 34.8 | 1.6 | 2.1 |
| 1 | 15 | 34.1 | 0.0 | 6.8 |
| 1 | 20 | 33.0 | 0.0 | 2.0 |
| 1 | 25 | 34.1 | 0.0 | 6.3 |
| 1 | 30 | 33.9 | 0.0 | 6.3 |
| 3 | 0 | 58.1 | 61.2 | 14.4 |
| 3 | 5 | 57.4 | 40.4 | 10.6 |
| 3 | 10 | 51.7 | 2.0 | 8.7 |
| 3 | 15 | 49.9 | 0.0 | 8.4 |
| 3 | 25 | 50.5 | 0.0 | 8.3 |
| 3 | 30 | 49.8 | 0.0 | 8.3 |
| 5 | 0 | 25.4 | 11.8 | 31.5 |
| 5 | 5 | 26.9 | 10.4 | 18.5 |
| 5 | 10 | 24.0 | 0.4 | 17.1 |
| 5 | 15 | 23.2 | 0.0 | 16.1 |
| 5 | 20 | 23.8 | 0.0 | 15.6 |
| 5 | 25 | 23.7 | 0.0 | 16.2 |
| 5 | 30 | 24.4 | 0.0 | 16.6 |
| 7 | 0 | 8.6 | 1.0 | 60.6 |
| 7 | 5 | 9.3 | 1.5 | 37.0 |
| 7 | 10 | 8.0 | 0.0 | 41.8 |
| 7 | 15 | 8.2 | 0.0 | 27.8 |
| 7 | 20 | 8.0 | 0.0 | 25.8 |
| 7 | 25 | 8.0 | 0.0 | 22.9 |
| 7 | 30 | 8.4 | 0.0 | 24.8 |
| 9 | 0 | 2.4 | 0.05 | 107.3 |
| 9 | 5 | 3.0 | 0.1 | 55.6 |
| 9 | 10 | 2.2 | 0.0 | 41.2 |
| 9 | 15 | 2.2 | 0.0 | 39.4 |
| 9 | 20 | 2.4 | 0.0 | 38.8 |
| 9 | 25 | 2.5 | 0.0 | 36.6 |
| 9 | 30 | 2.3 | 0.0 | 37.4 |

A number of things are apparent from these results. Firstly, phrap is able to reconstruct mutants 2 to 3 times faster than it is able to reconstruct the original sequence, regardless of the level of coverage. Secondly, for low coverage levels, phrap is much more likely to incorrectly join two fragments of the original sequence than to incorrectly join two fragments of a mutant. A substitution level of 10% appears to be sufficient to deliver most of the benefits of mutation. Higher substitution levels deliver little apparent improvement.

A number of things should be stated about these results. Firstly, they pertain to a single 120 kb sequence and might not be indicative of the performance of shotgun reconstruction algorithms on other sequences. In particular, the effect of introducing mutations is likely to be quantitatively different for sequences that are more repetitive than the one used here. However, the same qualitative trends are expected. Secondly, these simulations do NOT represent simulated SAM reconstructions. They merely illustrate how the introduction of mutations can facilitate the reconstruction process.

### Example 7

Application of SAM to Target Sequences Having Different Rates of Mutation

This example summarizes the results of applying the sequence reconstruction algorithms to target fragments that have undergone computer simulations of various rates of mutation. In each case, around 1000 target fragments were chosen at random from a database of genomic DNA, and a number of mutant copies of each target was obtained computationally. Mutation was random, with given probabilities of inserting a new base randomly at each location in the string, deleting a given base from the string, and substituting an existing base with a new (random) base. Levels of mutation were chosen which may be representative of what could be attained under laboratory conditions, but the principles demonstrated by the results are not dependent on the particular mutation model chosen.

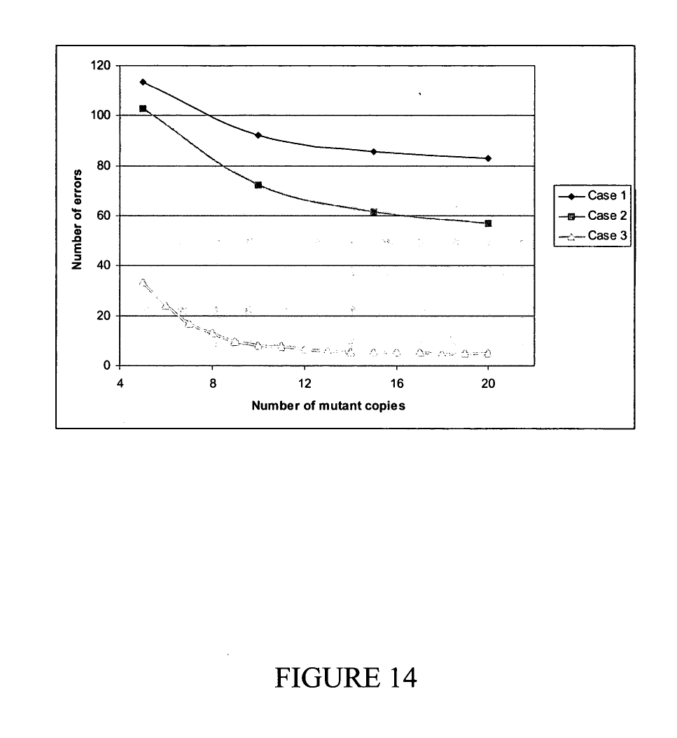

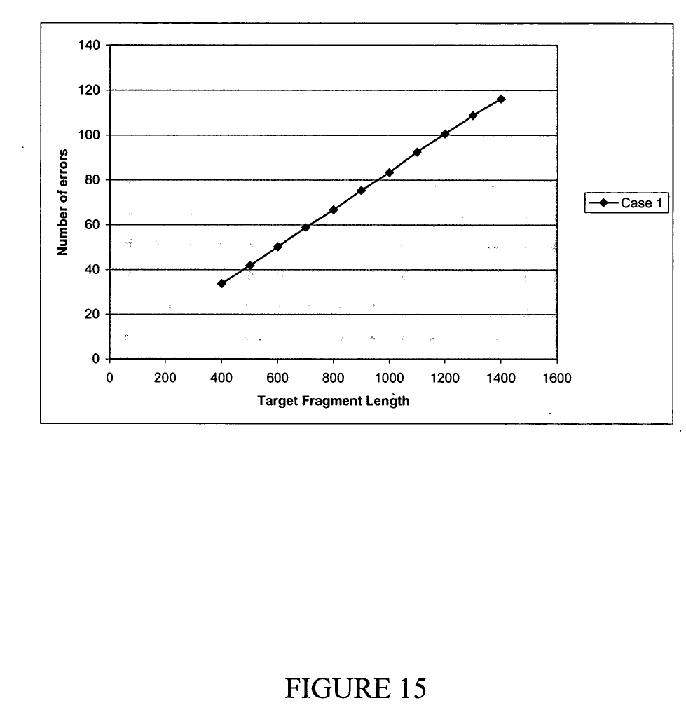

The public-domain software package clustalW was used to create a consensus string from the mutant copies, the consensus string was compared to the original target fragment, and the number of bases in which the target fragment and the consensus string differed were counted. This number is called the Number of errors, and an average was obtained over the 1000 simulations for each level of mutation. It is the average results that are presented in FIGS. 14 and 15.

FIG. 14 shows, for target fragments of length 400, the number of errors that occurred as the number of mutant copies was increased. As expected, the number of errors reduced as the number of mutant copies increased. Case 1 in FIG. 14 represents random mutation with probability of insertion of a base 10%, probability of deletion 10% and probability of substitution 10%. Case 2 in FIG. 14 represents probabilities of insertion, deletion and substitution of 5%, 5% and 20% respectively, and Case 3 in FIG. 14 represents 1%, 1% and 20% respectively.

FIG. 15 shows the number of errors that occurred as the target fragment length was increased. As expected, the number of errors increases linearly with target fragment length. Mutation was random, with probability of insertion of a base 1%, probability of deletion 1% and probability of substitution 20%.

Tables 4 and 5 show the number of errors in the reconstructed string for various levels of mutation, number of mutants and target fragment lengths. Probabilities of insertion of a base, deletion of a base and substitution of a base with a new base are given in the first three columns. The number of mutant copies is given in the fourth column, and the average number of errors in the reconstructed string is given in the final column. Table 4 has target fragments of length 400, and Table 5 has target fragments of length 2000.

TABLE 4
Average number of errors in the reconstructed string for target
fragments on length 400 and various levels of mutation.

| | | | Num. | Number |
|---|---|---|---|---|
| P(insertion) | P(deletion) | P(substitution) | mutants | errors |
| 0.01 | 0.01 | 0.20 | 5 | 33.72 |
| 0.01 | 0.01 | 0.10 | 5 | 7.81 |
| 0.05 | 0.05 | 0.20 | 5 | 102.78 |
| 0.10 | 0.10 | 0.10 | 5 | 113.32 |
| 0.01 | 0.01 | 0.20 | 20 | 4.85 |
| 0.01 | 0.01 | 0.10 | 20 | 1.77 |
| 0.05 | 0.05 | 0.20 | 20 | 57.13 |
| 0.10 | 0.10 | 0.10 | 20 | 82.80 |

TABLE 5
Average number of errors in the reconstructed string for target
fragments on length 2000 and various levels of mutation.

| | | | Num. | Number |
|---|---|---|---|---|
| P(insertion) | P(deletion) | P(substitution) | mutants | errors |
| 0.01 | 0.01 | 0.20 | 5 | 167.71 |
| 0.01 | 0.01 | 0.10 | 5 | 38.93 |
| 0.05 | 0.05 | 0.20 | 5 | 517.73 |
| 0.10 | 0.10 | 0.10 | 5 | 568.37 |
| 0.01 | 0.01 | 0.20 | 20 | 24.42 |
| 0.01 | 0.01 | 0.10 | 20 | 8.68 |
| 0.05 | 0.05 | 0.20 | 20 | 283.40 |
| 0.10 | 0.10 | 0.10 | 20 | 418.23 |

### Example 8

Sequencing by Hybridization

A major limitation of current Sequencing by Hybridization (SBH) technology is the occurrence of reconstruction ambiguities, which arise due to repeated subsequences in the target DNA fragment. The mutation process used in SAM has great potential to disrupt the repeat structure, thus allowing longer fragments to be uniquely reconstructed using given SBH probe lengths. We have conducted some preliminary investigations of the reconstructability of mutated fragments compared to reconstructability of the target fragment.

Investigations were undertaken on the laboratory mutated DNA fragments and the original fragment described in Example 3. Sub-fragments of various lengths were selected at random from these fragments, and subjected to a simulated SBH experiments with various probe lengths. The SBH spectrum was then used for reconstruction of the corresponding fragment, with the proportion of unique reconstructions being measured. This is clearly a small data set, but the results (shown in Table 6) demonstrate clearly the improved performance of SBH on the mutated fragments (column 5) when compared to the non-mutated data (column 4). Note that Case 1, Case 2 and Case 3 of Table 5 correspond to three types of SBH experiment: one in which hybridization reveals all subsequences and their frequency of occurrence (Case 3); one in which hybridization reveals all subsequences and the total fragment length (Case 2); and one in which hybridization reveals all subsequences but no further information.

TABLE 6
Proportion of various fragments with unique reconstruction
using SBH with various probe lengths.

| | | | Propor- | Propor- |
|---|---|---|---|---|
| | | | tion with | tion with |
| | | | unique | unique |
| Type | | | recon- | recon- |
| of SBH | Frag- | SBH | struction | struction |
| exper- | ment | Probe | (target | (mutated |
| iment | length | length | fragment) | fragments) |
| Case 1 | 100 | 6 | 0% | 0% |
| Case 2 | 100 | 6 | 0% | 30% |
| Case 3 | 100 | 6 | 0% | 30% |
| Case 1 | 100 | 7 | 10% | 55% |
| Case 2 | 100 | 7 | 70% | 98% |
| Case 3 | 100 | 7 | 70% | 98% |
| Case 1 | 100 | 8 | 60% | 70% |
| Case 2 | 100 | 8 | 100% | 100% |
| Case 3 | 100 | 8 | 100% | 100% |
| Case 1 | 130 | 7 | 0% | 23% |
| Case 2 | 130 | 7 | 70% | 93% |
| Case 3 | 130 | 7 | 70% | 93% |

### Example 9

DNA Folding Simulations

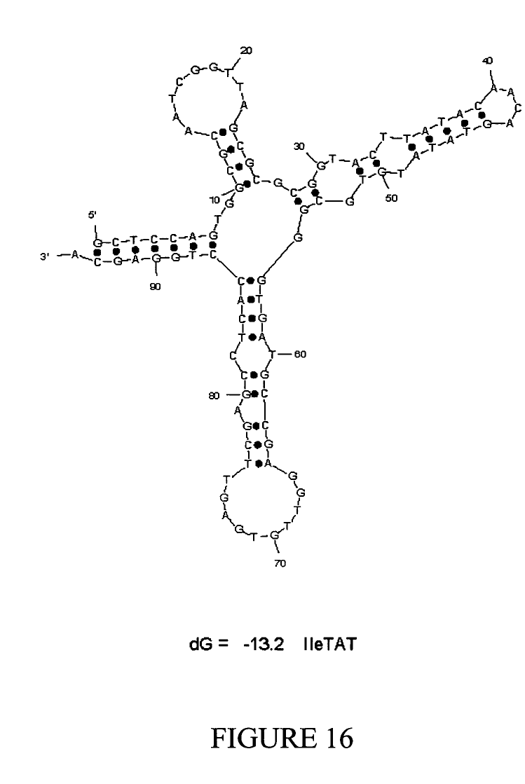

The following tests are intended to illustrate that introduced mutations can disrupt secondary structures in DNA molecules, thus rendering the molecules less stable and presumably easier to sequence. The tests were conducted using the DNA complement of a tRNA molecule. It was selected because TRNA has an interesting ‘clover-leaf’ secondary structure. Although the DNA complement does not form the same structure as the tRNA, it nevertheless forms a secondary structure that provides an interesting test of the SAM concept. The sequence of the DNA was obtained from GtRDB (Genomic tRNA Data Base) at http://rna.wustl.edu/tRNAdb/ and is shown below. FIG. 16 illustrates an indication of the structure it folds to, as determined by the mfold server at http://bioinfo.math.rpi.edu/˜zukerm/

| | |
|---|---|
| GCTCCAGTGGCGCAATCGGTTAGCGCGCGGTACTTATACAACAGTATATGTGCGGGTGA | [SEQ ID NO:188] |
| TGCCGAGGTTGTGAGTTCGAGCCTCACCTGGAGCA | |

The above DNA does not cause major sequencing difficulties. Nevertheless, these tests do illustrate the plausibility of disrupting secondary structure via mutation. Two sets of twenty mutants of the above sequence were generated in silico. In the first set of mutants, the probability of any particular base being substituted was 0.2, and where substitutions occurred the new base was selected with equal probability from the three alternatives. In the second set of mutants, the probability of any particular base being substituted was also 0.2, but the only allowed substitutions were transitions (A to G, G to A, T to C and C to T). This second set models a constraint that occurs with some types of mutagenesis. For comparison, 20 random sequences of the same length (94 nt) were also generated. The free energies of the folded molecules, as determined by mfold, are shown in Table 7.

TABLE 7
Free energies of the folded mutant tRNA molecules

| All | Transitions | Random |
|---|---|---|
| Substitutions | only | Sequences |
| −10.1 | −8 | −5.3 |
| −13.1 | −6.6 | −4.8 |
| −10.2 | −10 | −12.1 |
| −12.2 | −13.4 | −7.9 |
| −11.9 | −12.6 | −9.2 |
| −13 | −12.3 | −7.9 |
| −8.5 | −17.4 | −9.6 |
| −9.6 | −12.1 | −8.9 |
| −13.8 | −10 | −6 |
| −11.2 | −13.4 | −5.5 |
| −9.1 | −11.4 | −6.7 |
| −9.9 | −8.7 | −4 |
| −11.5 | −9.9 | −8.3 |
| −9.5 | −10.2 | −11.5 |
| −12.2 | −13 | −6.7 |
| −8.6 | −12.3 | −7.3 |
| −7.5 | −9.6 | −7 |
| −10.3 | −11.5 | −8.7 |
| −15 | −8.1 | −5.6 |
| −13.6 | −10.1 | −8 |

The average free energies of the mutant molecules were −11.04 and −11.03 kcal/mol for the two groups respectively and the average free energy of the random molecules was −7.55 kcal/mol. Compare this to the free energy of −13.16 kcal/mol for the original molecule. Three things are apparent from this test. Firstly, the average energies of the mutants are higher than that of the original molecule, indicating that the mutants are less stable and therefore likely to be easier to sequence. Secondly, there is no significant difference between the average energies of the two mutant data sets, and hence constraining the substitutions to be transitions only does not appear to make a difference to the ability of mutation to disrupt secondary structure. Thirdly, the mutants are still significantly more stable than random sequences.

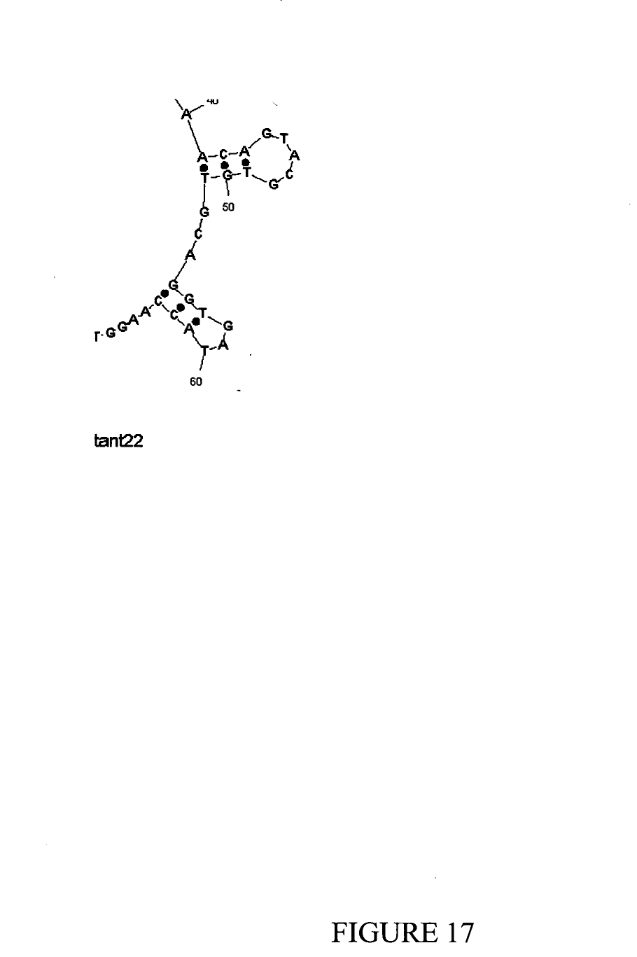

An indication of the secondary structure of the highest energy mutant, as predicted by mfold, designated mutant 22, is presented in FIG. 17.

### Example 10

SAM Applied to Shotgun Sequencing II

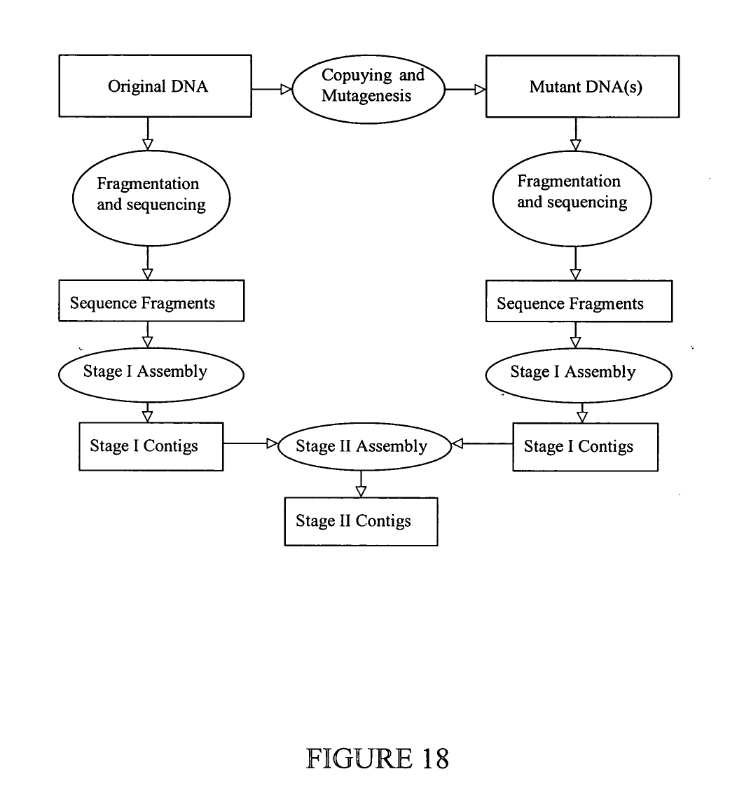

Three paradigms for the application of SAM in shotgun sequencing are illustrated in flow-chart form in FIGS. 18 to 20, respectively. The scale of the original DNA is not mentioned, and the diagrams and discussion here are intended to be sufficiently general to refer either to whole-genome shotgun sequencing or clone-length shotgun sequencing.

In the first paradigm (FIG. 18), mutants are processed separately from each other and from the original DNA molecule, up until stage II assembly (see below). FIG. 18 shows only one mutant but in general several mutants could be processed in parallel. The first stage of the process is generation of the mutants: The next stage —‘Fragmentation and sequencing’ may involve many sub-steps including cloning, sub-cloning and PCR. Stage I assembly involves a cautious assembly of the fragments into contigs. Fragments from different mutants are not joined to each other at this stage, or to fragments of the original sequence. The assembly is cautious because mistakes made at this stage will make Stage II assembly more difficult. Stage II assembly involves merging the contigs and fragments output by the stage I assembly to infer longer contigs of the original sequence (and incidentally of the mutants). Stage II assembly may involve taking a consensus of mutant sequences in places where fragments of the original sequence are not available.

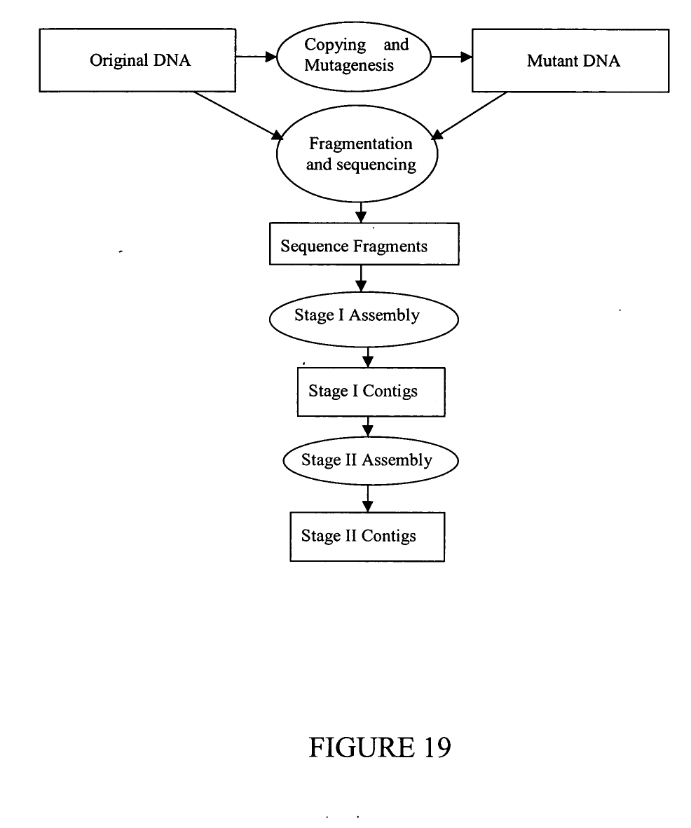

In the second paradigm (FIG. 19), the mutants are not kept separate from the original sequence. Fragments of sequence are obtained from the mutants and from the original, but the origin of individual fragments is not available. In Stage I assembly, fragments are assembled into contigs, but the goal at this stage is to avoid joining fragments from different mutants or from the original sequence and a mutant. This is possible because overlapping fragments from the same mutant (or from the original) are more similar to each other than overlapping fragments from different sources. In Stage II assembly, the contigs and fragments formed by the phase I assembly are merged and used to infer longer contigs of the original sequence. Again, this may involve taking a consensus of mutants in certain parts of the sequence.

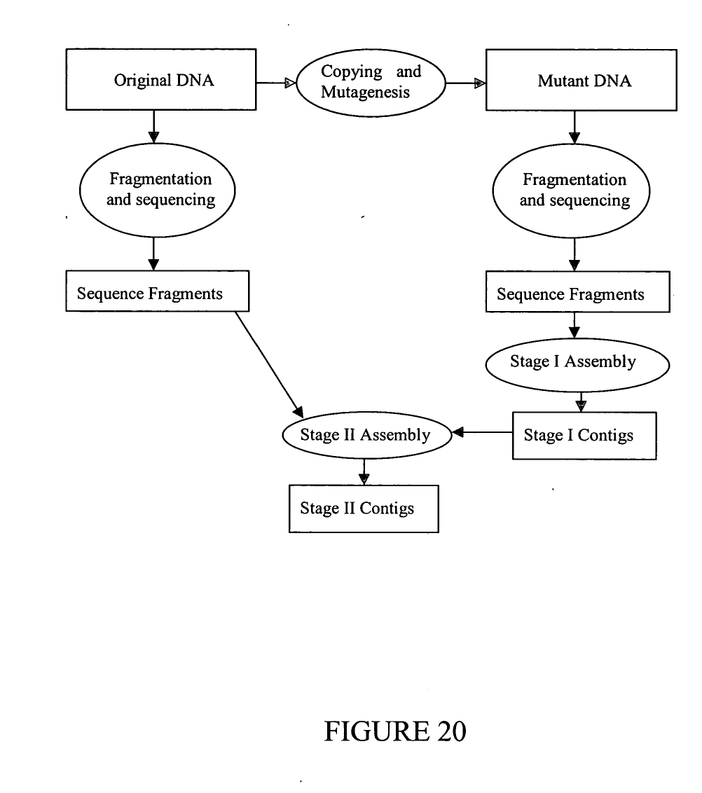

In the third paradigm (FIG. 20), the contigs of the original sequence are not assembled until stage II, after contigs of the mutants have been assembled. These mutant contigs are then used to assist in the assembly of contigs of the original sequence.

Options and Variations

Several options and variations are available for the above procedure. Firstly, the coverage of the original sequence and the various mutants need not be equal. Where possible, the coverage of the original DNA should be larger than that of the mutants, since sequences taken from the original are a better guide to the true sequence due to the absence of mutations.

In some cases, sequences taken from the original DNA may not be available. In such cases, the stages on the left of FIG. 18 from ‘Fragmentation and Sequencing’ through to ‘Stage I Contigs’ would not apply. Similarly, in FIG. 19 the arrow joining ‘Original DNA’ to ‘Fragmentation and Sequencing’ would not apply. Note that in FIG. 18 the data from individual mutants would still be processed independently up until Stage II assembly.

The algorithms that have been developed by the inventors so far involve two stages of assembly as shown in FIGS. 18 to 20. However, it is conceivable (and perhaps even preferable) that the two stages of assembly could be merged. The idea would be to arrange all of the fragments relative to each other, without keeping contigs of the mutants separate from each other and from contigs of the original sequence in a preliminary stage. Such a procedure would avoid problems caused by making mistakes in the stage I assembly.

The term ‘assembly’ in the above discussion and in FIGS. 18 and 20 should not be taken as limiting the SAM shotgun algorithms to those based on alignment and it is conceivable in this regard that sequences could be assembled in the absence of alignment. Thus, it will be understood that the term, ‘assembly’ should be interpreted broadly to include ‘finding a sequence or sequence profile consistent with the data available’.

Benefits of SAM in Shotgun

SAM provides several benefits in shotgun sequencing. Firstly, sections of the DNA that are refractory to the method of cloning and/or sequencing can be rendered amenable to these processes by introducing mutations. Secondly, introduced mutations facilitate the assembly stage of shotgun assembly by removing much of the ambiguity caused by repetitive sequence. In Example 6, results of simulations are presented, showing that the shotgun reconstruction software ‘phrap’ was able to assemble mutant DNAs more accurately and rapidly than it could assemble the original DNAs from which they were derived. Contigs that can be unambiguously reconstructed for the mutants can be used as templates to help resolve ambiguities in the reconstruction of the original sequence.

### Example 11

SAM Applied to Shotgun Sequencing III

Here a simulated example of shotgun reconstruction is presented, which uses SAM and phrap. A 120 kb DNA sequence was obtained from GenBank (Accession number AC000003). Ten mutants of this sequence were generated in silico. In each mutant, the probability of any particular base being modified was 10%. Shotgun sequencing with 1-fold coverage was simulated for each mutant. Sequencing errors were not simulated.

The fragments obtained from the mutants were kept separate as in the first of the three paradigms for SAM shotgun discussed in Example 10. Phrap was used to perform stage I assemblies. That is, the fragments for each mutant were independently assembled into contigs. These contigs and any fragments that were not put into contigs in Stage I were then pooled in a single file ready for Stage II assembly. Phrap was again used to assemble the pooled sequences. The output from phrap was processed using our own software to generate consensus sequences because phrap generates a mosaic sequence rather than a consensus sequence.

This process produced two contigs of length 59611 and 62667 bases respectively. Comparing this assembly to the correct assembly, it was found that a single join was missed; there would have been only one contig in a perfect assembly. No incorrect joins were made. The consensus sequences of the reconstructed contigs differed from the true sequences of these contigs in about 0.7% of bases. This is quite remarkable, given that no fragments of the original sequence were used at any stage of the assembly process.

In the Stage I assemblies, phrap was instructed not to allow any mismatches, insertions or deletions in its assembly. This was feasible only because we did not simulate sequencing errors. In the Stage II assembly, phrap was instructed not to allow insertions or deletions. This was feasible only because the mutations were substitutions only.

Algorithm

In this example, an algorithm is described for shotgun reconstruction of a clone-length DNA using SAM. The input to the algorithm consists of sequences of fragments of a number of mutants, and optionally sequences of fragments of the original DNA. In this algorithm, the sequences obtained from the mutants are kept separate from each other and from sequences obtained from the original DNA in a first stage of reconstruction, as in the first of the paradigms described in Example 10 and FIG. 18. The algorithm consist of the following steps:

a) Independent assembly of the sequences obtained from each individual mutant and independent assembly of the sequences obtained from the original DNA using phrap. The input parameters of phrap are tuned to inhibit making doubtful joins.

b) Pooling of the contigs and singlets output in step a) in a single file.

c) Second stage assembly of the pooled contigs and singlets using phrap. The input parameters of phrap are tuned to allow joins in which the overlapping segments are substantially different.

d) Processing of phrap output to determine consensus contigs.

e) Output of consensus contigs.

### EXAMPLE 12

Estimating Sequence Length Using SAM

The following example is an application of SAM that does not involve sequence alignment. Consider a DNA molecule of unknown length. One way to estimate the length of the molecule is the following procedure. First generate a number of mutant DNAs differing from the original molecule by one or more insertions, deletions and/or substitutions. Then measure the length of the mutant DNAs in some manner. Provided that the mutagenesis techniques used to produce the mutants are equally likely to result in an insertion or a deletion, the average length of the mutant DNAs may be taken as an estimate of the length of the original sequence. The purpose of this example is merely to illustrate that the SAM technique can be used in applications other than those directed to sequence analysis.

### Example 13

Biased PCR

The target sequence was an undefined DNA fragment of 1.5 Kb in length cloned into pUC19. Universal M13 primers (FSP-21, FSP-40, RSP-26, RSP-48) were used for PCR amplification and sequencing. Amplification conditions were essentially as described by Vartanian et al. (1996, Nucleic Acids Research 24(14): 2627-2631). Using a dNTP substrate pool biased in the concentrations of nucleotides, misincorporation of dNTPs was achieved resulting in a mutation frequency of 1 in 20 using a standard PCR reaction.

Reaction Conditions:

2 ng DNA template, 1× AmpliTaq Gold buffer, 1 mM magnesium chloride, 0.4 [symbol] M each primer, 1 unit of AmpliTaq Gold, in a total of 25 [symbol] L. The dNTP concentration were: 75 ωM dCTP, 1 mM dTTP, 200 [symbol] M dATP, and 200 [symbol] M dGTP. Reactions were performed as follows: 1 cycle of 94° C. for 15 min., 30 cycles of 94° C. 1 min, 50° C., 0.5 min, 72° C. 5 min., 1 cycle 72° C. 10 min.

Cloning of PCR Products and Sequencing:

The mutant PCR products were gel purified and cloned into the pGEM T-EASY vector (Promega) and transformed into E. coli. Plasmids DNA from individual clones were sequenced and analyzed for mutation frequency.

### Example 14

Nucleotide Analogue PCR

The target sequence was an undefined DNA fragment of 1.5 Kb in length cloned into pUC19. Universal M13 primers (FSP-21, FSP-40, RSP-26, RSP-48) were used for PCR amplification and sequencing. Amplification conditions were essentially as described by Zaccolo et al. (1996, Journal of Molecular Biology 255: 589-603). Using a concentration of 400 μM of each dATP, dCTP, dGTP, dTTP, and either dPTP or 8-oxo-GTP in a standard PCR reaction mutations were incorporated up to a frequency of 1 in 5. The action of the analogues was investigated when used individually or together.

Reaction Conditions:

2 ng DNA template, 1× AmpliTaq Gold buffer, 400 [symbol] M dNTPs, 2 mM magnesium chloride, 0.4 [symbol] M each primer, 1 unit of AmpliTaq Gold, in a total of 25 [symbol] L. Reactions were performed as follows: 1 cycle of 94° C. for 15 min., 30 cycles of 94° C. 1 min, 50° C., 0.5 min, 72° C. for 5 min., 1 cycle 72° C. for 10 min. This regimen yields PCR products incorporating analogue bases. Viable mutated DNAs were recovered by re-amplification of 1 [symbol] L of the reaction products with nested primers and the four natural dNTPs and standard PCR conditions.

Cloning of PCR Products and Sequencing:

The mutant PCR products were gel purified and then cloned into the pGEM T-EASY vector (Promega) and transformed into E. coli. Plasmids DNA from individual clones were sequenced and analyzed for mutation frequency.

### Example 15

DNA Synthesis In Vitro Using DNA Polymerase Extension (Klenow Fragment)

The following protocol is based on methods described by Kaminya and Kasai (1995, Journal of Biological Chemistry 270: 19,446) and Permal et al., 1994, Nucleic Acids Research 22: 3930).

1. DNA templates were annealed with primer in a buffer with:

DNA-primer complex (0.50 pM=0.05 μM)

50 mM Tris-HCl pH 8.0,

8.0 mM MgCl₂,

5.0 mM b-mercaptoethanol,

0.2 mg/ml BSA

50 [symbol] M dNTPs (50 μM of 3 dNTPs and 50 μM of fourth the oxidized-dNTP)

0.1 Units Klenow Fragment DNA polymerase enzyme in a total volume of 10

[symbol] L at 25° C. for 120 min.

2. The efficiencies of Klenow Fragment catalyzed incorporation of 2-OH-dATP and 8-OH-dGTP are similar.

### Example 16

Chemical Mutagenesis

The following protocols are based on methods disclosed by Walton et al. (1991, Random chemical mutagenesis and the non-selective isolation of mutated DNA sequences in vitro, in ‘Directed Mutagenesis—a practical approach’ (ed, M. J. McPherson), IRL Press) and Myers et al. (1985, Science 229: 242-247).

General:

Mutagenic chemicals are much more active with single stranded DNA than duplex DNA. The protocols below there will be on average ONE HIT per 1500 bp in 10 mins. Thus for a 150 bp fragment only 10% would be hit once in 10 min, as the longer the molecule the greater the likelihood of at least one hit. The second strand is regenerated by use of AMV Reverse transcriptase.

Protocol 1:

Depurination of DNA by Mild Acid Hydrolysis with Formic Acid

1. Adjust the concentration of ssDNA to 1 mg/ml with TE and place 40 μL in an Eppendorf tube.

2. Add 60 μl of concentrated 18M formic acid and mix well

3. Incubate the reaction at room temperature for a time at which will result in approximately 10% of the target fragments being hit.

4. Stop the reaction by quenching with:

200 μL 2.5M sodium acetate (pH 5.5)

100 μL H₂O

20 μg tRNA and

1 ml of cold Ethanol.

5. Precipitate the DNA by chilling at −70° C. for 30 min.

Protocol 2:

Depyrimidation of DNA by Hydrazine

1. Adjust the concentration of ssDNA to 1 mg/mL with TE and place 40 PL in an Eppendorf tube. Make sure strong buffers are not present—they inhibit the reaction of hydrazine with thymidine.

2. Add 60 μL of concentrated 12 M hydrazine and mix well.

3. Incubate the reaction at room temperature for a time at which will result in approximately 10% of the target fragments being hit—assume the same reactivity as for formic acid.

4. Stop the reaction by quenching with:

200 μL 2.5M sodium acetate (pH 5.5),

100 μL H₂O,

20 μg tRNA and

1.0 mL of cold Ethanol.

5. Precipitate the DNA by chilling at −70° C. for 30 min.

6. The DNA is reprecipitated TWO MORE times to remove all traces of the mutagen

Nitrous acid: is prepared by 1 hr reaction in 250 mM sodium acetate, pH 4.3 and 1.0 M sodium nitrite. A stock of 2M sodium nitrite is made up in water and stored at 4° C. for max 1 week.

Permanganate: is prepared by 10 min treatment with 0.13 M potassium permanganate results in approx. 10% mutagenesis

| | Primary Targets |
|---|---|
| Nitrous acid: | C, A, G |
| Formic acid: | G, A |
| Hydrazine: | C, T |
| Permanganate: | C, T |

Protocol 3:

Second Strand Synthesis Using AMV Reverse Transcriptase.

Many Reverse Transcriptases (RT) lack ‘editing functions’ and thus are not stopped when they encounter a missing or damaged base. AMV RT however cannot initiate synthesis de novo and can only extend 3′-hydroxy termini of existing chains. AMV-RT is also error prone and will incorporate one error [mismatched] base per 600 polymerized. These mismatch errors will be repaired if the duplex DNA product is then cloned into wild-type E. coli—hence mutant bacterial strains must be used as hosts if high levels of modification are to be found.

1. Remove all last traces of the mutagen from the precipitated DNA by redissolving in 10 mL Tris-HCl, pH 7.5, 100 mM NaCl, 1 mM EDTA, and 20 [symbol] g/mL tRNA. Re-precipitate DNA by adding 2× volumes of cold ethanol.

### Example 17

Oxidation of DNA Using Hydrogen Peroxide

Hydrogen peroxide-mediated mutagenesis of DNA can be carried out using methods as for example disclosed by Kaminya & Kasai (1995, Journal of Biological Chemistry 270: 19,446), Feig et al. (1994, Proceedings of the National Academy of Sciences USA 91: 6609), Permal et al. (1994, Nucleic Acids Research 22: 3930) and Cheng et al. (1992, Journal of Biological Chemistry 267: 166).

### Example 18

Bisulphite Treatment

The following protocols is based on the method disclosed by Feil et al. (1994, Nucleic Acids Research 22: 695-696)

Solutions:

3.5M NaHSO₃/1 mM Hydroquinone Solution:

Dissolve 8.1 g of Sigma mix in 18 mL water, adjust to pH 5.0 with 5 M NaOH,

Add 1 ml of 20 mM hydroquinone then make volume to final total of 20 mL.

Complete Sulphonation Reactions:

1. Genomic DNA (2 [symbol] g) is digested with endonuclease to give a small fragment containing the sequence of interest.

2. The digestate is phenol-extracted twice to remove protein, then ethanol precipitated and the resulting pellet dissolved in 100 [symbol] L H₂O in a silanized Eppendorf tube.

3. DNA is denatured by adding 11 [symbol] L 3M NaOH, incubate 37° C. for 20 min.

4. The Eppendorf tube is placed on ice, and 1.1 mL of 3.5M NaHSO₃/1 mM hydroquinone, pH 5.0 is added.

5. Aqueous solution is overlaid with 150 [symbol] L mineral oil and incubated in dark for 24 hr* at 0° C. * For partial/incomplete reaction incubate for shorter periods.

6. The solution extracted under the oil and transferred into a fresh silanized tube at 0° C.

7. DNA is extracted from solution at 0° C. in dark with 20 [symbol] L glass milk (GeneClean II.

8. The glass breads are washed 3× with GeneClean new wash, then air dried.

9. The DNA is dissolved in 100 [symbol] L water and stored at −20° C. until use.

Desulphonation of DNA:

1. Desulphonation is performed by adding 11 [symbol] L of 2N NaOH (final 0.2 M) followed by incubation at 20° C. for 10 min.

2. SM Sodium acetate (pH 7.0) is added to a final concentration of 3 M and the DNA precipitated with 3× volumes of EtOH.

### Example 19

Hydrogen Peroxide Treatment:

| Solvents | Aqueous | | 80% Formamide | |
|---|---|---|---|---|
| ddH₂O | 352 | L | 42/38 | [symbol] L |
| Formamide | 0 | [symbol] L | 320 | [symbol] L |
| DNA: Whole | 4 | [symbol] L | 4 | [symbol] L |
| lambda, | | | | |
| plasmids | | | | |
| 3.0 M | *15.0 | [symbol] L | *15.0 | [symbol] L |
| Sodium acetate, | | | | |
| pH 8.55 | | | | |
| 1 M Ascorbate | *8.0 | [symbol] L | *8.0 | [symbol] L |
| in H₂O | | | | |
| 1.76 M H₂O₂ | 15.0 | [symbol] L | 1.0 | [symbol] L |
| 1.0 mM | *10.0 [symbol] M = +/−4.0 [symbol] L | | *2.5 μM = +/−1.0 [symbol] L | |
| NiCl₂/*Optional | | | | |
| TOTAL | 400 | [symbol] L | 400 | [symbol] L |

| | | |
|---|---|---|
| Reaction | 40° C. for 10-60 min | 20° C. (RT) for 1-10 min |
| conditions | | |

The reaction is terminated by addition of 0.2× volume of 3.0 M Sodium acetate, pH 5.0 and 3.0× volumes of ice cold ethanol, which results in the precipitation of the DNA. The DNA is then collected by centrifugation for 15 min at 10,000 g, washing the resulting pellet with 70% Ethanol and recentrifiging before drying the pellet and dissolving in H₂O.

### Example 20

Hydrazine Treatment:

| | | | 80% | |
|---|---|---|---|---|
| | Aqueous | | formamide | |
| | and | | and | |
| Solvents | Hydrazine | | Hydrazine | |
| ddH₂O | 310 | L | 0 | [symbol] L |
| Formamide | 0 | [symbol] L | 310 | [symbol] L |
| Hydrazine | 80 | [symbol] L | 80 | [symbol] L |
| DNAs: | 6 | [symbol] L | 6 | [symbol] L |
| Whole Lambda, | | | | |
| pGEM | | | | |
| 3.0 M | 150 mM @ pH 7.0 | | 150 mM @ pH 7.0 | |
| Sodium | | | | |
| acetate, | | | | |
| pH 7.0 | | | | |
| TOTAL | 400 | [symbol] L | 400 | [symbol] L |

| | | |
|---|---|---|
| Reaction | 20 C (RT) for 1-10 min | ICE for 1-10 min |
| conditions | | |

The reaction is terminated by addition of 0.2× volume of 3.0 M Sodium acetate, pH 5.0 and 3.0× volumes of ice cold ethanol, which results in the precipitation of the DNA. The DNA is then collected by centrifugation for 15 min at 10,000 g, washing the resulting pellet with 70% Ethanol and recentrifuging before drying the pellet and dissolving in H₂O.

### Example 21

Formic Acid Treatment:

| Final concentration Vol = 100 [symbol] L | Aqueous 4 × volume |
|---|---|
| dd H₂O | 384 [symbol] L |
| Formic acid → | 8 [symbol] L |
| 3 M Sod Ac pH 7.0 | 2 [symbol] L with tRNA |
| DNA Whole lambda at 6 ng/ [symbol] L | 6 [symbol] L |
| TOTAL | 400 [symbol] L |
| Reaction conditions | ON ICE: 1-10 min |

The reaction is terminated by addition of 0.2× volume of 3.0 M Sodium acetate, pH 5.0 and 3.0× volumes of ice cold ethanol, which results in the precipitation of the DNA. The DNA is then collected by centrifugation for 15 min at 10,000 g, washing the resulting pellet with 70% Ethanol and recentrifuging before drying the pellet and dissolving in H₂O.

### Example 22

Repair Deficient E. coli Strains/Genotypes

| | | | DEFINED REPAIR |
|---|---|---|---|
| STRAIN | RESISTANCE | MUTANT GENE | SYSTEM |
| XL1-Red | Tet | MutS = mismatch | =Methyl-directed |
| competent | | repair | mismatch repair |
| | | MutD = e sbunit pol | =Proof-red repair DNA |
| | | III | pol III |
| | | MutT = 8OH-dG | =Hydrolyzes 8-oxo- |
| | | hydrolase | dGTP |
| | | | VERY STRONG |
| | | | EFFECT |
| XL-mutS | Kan & Tet | MutS = mismatch | =Methyl-directed |
| competent | | repair | mismatch repair |
| | | | STRONG EFFECT |
| JM 105 = K12, rspL | Sm | rspL | — |
| BH 410 = JM105, | Kan, [Sm] | mut M = fpg-1 | Wild-type enzyme |
| fpg-1::Kan | | | removes d°GTP |
| | | | incorp/d opposite C |
| BH 420 = JM105, | Kan, Tet, [Sm] | mut M = fpg-1 | Wild-type enzyme |
| fpg-1::Kan, | | and | removes d°GTP incorp/d |
| uvrA::Tet | | UvrA | opposite C PLUS |
| | | | Nucleotide-excision |
| | | | repair (uvrA) |
| BH 430 = JM105, | Tet, [Sm] | UvrA | Nucleotide-excision |
| UvrA::Tet | | | repair (NER) (uvrA, |
| | | | uvrC) |
| BH 700 = JM105, | Kan [Sm] | UmuC | |
| UmuC::Kan | | | |
| BH 1040 = K12, | Kan, Tet, [Sm] | mut M = fpg-1 | Wild-type enzyme |
| fpg-1::Kan | | and | removes d°GTP incorp/d |
| Mut Y:: | | I. mut Y = 8)H-dG | opposite C, PLUS |
| | | glycosylase | |
| BW 313 = K12, | No resistances | Uracil - | Grow @ 25-30 C, |
| Ung-1, dut-1, thi, | | | LB + Thymidine |
| relA1 | | | Test @ 42° C. |
| BW 1034 = K12, | KAN, CAT | | NfiI = endonuclease V |
| nfi-1::cat, | | | Ung = uracil DNA |
| ung153::kan | | | glycosylase |
| BW 1138 = K12, | Kan^{R} | | Ung = uracil DNA |
| Ung-153::Kan | | | glycosylase |
| BW 1160 = K12, | Cat | | NfiI = endonuclease V |
| nfi-1::Cat | | | |
| BW 1161 = AB1157, | CAT, SM | | NFII = ENDONUCLEASE V |
| nfi-1::Cat | | | |
| WP2 | SM [?] | TRPE65, MALB15, | |
| | | LON-11, SULA1 | |
| WP2mutT = MutT::Km | KAN, SM | MUT T = | 8-oxo-dGTP removal (−ve) |
| | | | W.T. ENZYME STOPS |
| | | | D°GTP INCORP |
| | | | OPPOSITE A |
| AB1157 = wt parent | | II | IV. |
| CC101T = AB1157, | Kan^{R}, [Sm] | Mut T = | 8-oxo-dGTP removal (−ve) |
| MutT::Km | | | w.t. enzyme stops d°GTP |
| Original & LB plate | | | incorp opposite A |
| MK 603 = AB1157, | KAN, [SM] | MUT M = FPG-1 | WILD-TYPE ENZYME |
| Mut M::Km | | | REMOVES D°GTP |
| | | | INCORP/D OPPOSITE C |
| MS 23 = AB1157, | Sm | | Hypoxanthine-DNA |
| alkA::?? | | | glycosylase |
| MK 609 = AB1157, | | Mut Y | w.t. enzyme removes A |
| Mut Y::Tet | | | misincorp opposite d°GTP |
| MK 611 = AB1157, | KAN, TET | MUT M = FPG-1 | FAPY |
| Mut M::Kan | | Mut Y = glycosylase | GLYCOSYLASE |
| Mut Y::Tet | | | 8-OH-dG gylcosylase |
| GM31 = dcm6⁻ | II. Sm | III. dcm6 (−ve) | IV. Patch repair(−) |
| GM31 + pDCM72 | V. Cm, Sm | VI. Vsr (−ve) | VIII. vsr gene = very |
| (vsr⁻, dcm⁺, ung⁺) | | VII. dcm&ung (+ve) | short patch repair |
| | | | IX. Plasmid is patch |
| | | | repair +ve ?? |
| GME c5 | Kan^{R} | | X. DNA pol I |
| GME c6 | Kan^{R} | | DNA pol I |
| Q 771 = GC4468: | Sm^{R} | | Being (771/F- Δ(lac- |
| | | | argF), U169, rpsL179 |
| Q 1729 = AB1157, | Tet, Sm | | |
| srl300::Tn10, | | | |
| recA430 | | | |
| Q 1736 = GC4468, | Kan^{R}, Cm^{R}, Sm^{R} | | Double SOD, & FUR |
| ΔsodA, | | | mutant |
| sodB::Cmr, | | | |
| Δfur::Kan | | | |
| Q 1726 = GC4468, | Cm^{R}, Sm^{R} | | Double SOD mutant |
| ΔsodA, sodB::Cmr | | | |
| KY5 = parental | | | II. |
| NKJ 2002 | CM^{R} | Endonuclease III | ΔNTH::CM |
| NKJ 2003 | KAN^{R} | Endonuclease VIII | ΔNEI::KAN |
| NKJ 2004 | CM^{R}, KAN^{R} | EndonucleaseIII, | ΔNTH::CM, |
| | | Endonuclease VIII | ΔNEI::KAN |
| SW2-38 | CM^{R}, KAN^{R} | EndonucleaseIII, | ΔNTH::KAN, |
| | | Endonuclease VIII | ΔNEI::CM |

The disclosure of every patent, patent application, and publication cited herein is hereby incorporated herein by reference in its entirety.

The citation of any reference herein should not be construed as an admission that such reference is available as “Prior Art” to the instant application

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

Altshuler, D., Pollara, V. J., Cowles, C. R., van Etten, W. J., Baldwin, J., Linton, L., Lander, E. S. (2000). An SNP map of the human genome generated by reduced representation shotgun sequencing. Nature 407, 513-516.

Bains, W. (1991). Hybridization methods for DNA sequencing. Genomics 11, 294-301.

Bains, W. and Smith, G. C. (1988). A novel method for nucleic acid sequence determination. J. Theor. Biol. 135, 303-307.

Cheng, S., Chen, Y., Monforte, J. A., Higuchi, R. and Van Houten, B. (1995). Template integrity is essential for PCR amplification of 20- to 30-kb sequences from genomic DNA. PCR Meths. Appl. 4, 294-298.

Chissoe, S. L., Marra, M. A., Hillier, L., Brinkman, R., Wilson, R. K., Waterston, R. H. (1997). Representation of cloned genomic sequences in two sequencing vectors: correlation of DNA sequence and subclone distribution. Nucleic Acids Res., 25, 2960-2966.

Cocchia, M., Kouprina, N., Kim, S.-J., Larionov, V., Schlessinger, D. and Nagaraja, R. (2000). Recovery and potential utility of YACs as circular YACs/BACs. Nucleic Acids Res. 28, e81.

Drmanac, R., Labat, I., Brukner, I. and Crkvenjakov, R. (1989). Sequencing of megabase plus DNA by hybridization: theory of the method. Genomics 4, 114-128.

Drmanac, R. (2000). Universal DNA sequencing chips for the analysis of SNPs and splice variants. In: 2000 International Forum on Biochip Technologies: International Conference on Engineering and Technological Sciences 2000, Session 8. Beijing, China, Oct. 11-14, 2000. Tsingua University Press, Beijing.

Frieze, A. M., Preparata, F. P. and Upfal, E. (1999). Optimal reconstruction of a sequence from its probes. Journal of Computational Biology 6(3/4), 361-368.

Fromenty, B., Demeilliers, C., Mansouri, A. and Pessayre, D. (2000). Escherichia coli exonuclease III enhances long PCR amplification of damaged DNA templates. Nucleic Acids Res. [Methods On Line] 28, e50.

Green, P. (1997). Against a whole-genome shotgun. Genome Res. 7, 410-417.

Gunderson, K. L., Huang, X. C., Morris, M. S., Lipshutz, R. J., Lockhart, D. J. and Chee, M. S. (1998). Mutation detection by ligation to complete n-mer DNA arrays. Genome Res. 8, 1142-1153.

Khrapko, K. R., Lysov, Yu. P., Khorlin, A. A., Shick, V. V., Florentiev, V. L. and Mirzabekov, A. D. (1989). An oligonucleotide hybridization approach to DNA sequencing. FEBS Letters 256, 118-122.

Leppard, K. N. (1999). Mutagenesis of DNA virus genomes, in DNA Viruses: A Practical Approach Series 214 (ed., Alan J. Cann), IRL Press, Oxford.

Ling, M. F. and Robinson, B. H. (1997). Approaches to DNA mutagenesis: an overview. Anal. Biochem. 254, 157-178.

Lipshutz, R. J., Fodor, S. P. A., Gingeras, T. R. and Lockhart, D. J. High density synthetic oligonucleotide arrays. (1999). Nat. Genet. 21, 20-24.

Lysov, Y. P., Florentiev, V. L., Khorlyn, A. A., Khrapko, K. R., Shick, V. V. and Mirzabekov, A. D. (1988). DNA sequencing by hybridization with oligonucleotides, Doklady Academii Nauk SSSR, 303, 1508-1511.

Olek, A., Oswald, J. and Walter, J. (1996). A modified and improved method for bisulphite based cytosine methylation analysis. Nucleic Acids Res. 24, 5064-5066.

Pe'er, I. and Shamir, R. (2000). Spectrum alignment: efficient resequencing by hybridization. ISMB 8, 260-268.

Pevzner, P. A. and Lipshutz, R. J. (1994). Towards DNA sequencing chips. In: Mathematical Foundations of Computer Science, number 841: 19th International Symposium, FCS'94, Kosice, Slovakia, Aug. 22-26, 1994: proceedings/Igor Privara, Branislav Rovan, Peter Ruzicka, eds. Berlin; New York: Springer-Verlag.

Ramsay, G. (1998). DNA chips: State-of-the-art. Biotechnology 16, 40-44.

Siegel, A. F., van den Engh, G., Hood, L., Trask, B., Roach, J. C. (2000). Modelling the feasibility of whole genome shotgun sequencing using a pairwise end strategy. Genomics 68, 237-246.

Stomakhin, A. A., Vasiliskov, V. A., Timofeev, E., Schulga, D., Cotter, R. J., Mirzabekov, A. D. (2000). DNA sequence analysis by hybridization with oligonucleotide microchips: MALDI mass spectrometry identification of 5mers contiguously stacked to microchip oligonucleotides. Nucleic Acids Res. 28, 1193-1198.

Venter, J. C., Adams, M. D., Sutton, G. G., Kerlavage, A. R., Smith, H. O., Hunkapillar, M. (1998). Shotgun sequencing of the human genome. Science 280, 1540-1542.

Warnecke, P. M., Mann, J. R., Frommer, M. and Clark, S. J. (1998). Bisulphite sequencing in pre-implantation embryos: DNA methylation profile of the upstream region of the mouse imprinted H19 gene. Genomics 51, 182-190.

What is claimed is:

1. A method for wholly or partially deducing the sequence of a target subunit sequence, comprising:
providing the sequences of a plurality of variants, which are distinguished individually from the target subunit sequence by the addition, deletion and/or substitution of at least one subunit;
comparing the individual sequences of said variants with each other and optionally with a sequence derived from the target subunit sequence or from a sequence adjacent thereto to deduce a consensus sequence, which corresponds to all or part of the target subunit sequence.

2. The method of claim 1, wherein the comparison is effected using sequence alignment or probabilistic techniques.

3. The method of claim 1, wherein said target subunit sequence is refractory to sequence analysis.

4. The method of claim 1, wherein said subunit sequences are selected from amino acid sequences or nucleic acid sequences.

5. The method of claim 1, wherein said subunit sequences are selected from nucleic acid sequences.

6. A method for wholly or partially determining the sequence of a primary subunit sequence, said method comprising:
providing at least one secondary subunit sequence which varies from said primary sequence by the addition, deletion and/or substitution of at least one subunit; and
comparing overlapping subsequences corresponding to said at least one secondary subunit sequence and to said primary subunit sequence to reconstruct at least a portion of the primary subunit sequence.

7. The method of claim 6, wherein the overlapping subsequences have a length p and the primary subunit sequence comprises repeated sequences ofp−1.

8. The method of claim 7, wherein the variation in the at least one secondary subunit sequence is associated with the alteration or destruction of at least one of said repeated subsequences.

9. The method of claim 6, wherein said comparison comprises alternately reconstructing said primary subunit sequence and said at least one secondary subunit sequence using an end portion of a respective reconstruction as a guide to extend another reconstruction.

10. The method of claim 9, wherein said alternate reconstruction comprises:
comparing a portion of the primary subunit sequence with subsequences corresponding to said at least one secondary subunit sequence to identify a subsequence which aligns best with said portion and which extends unambiguously in said alignment a reconstruction of said at least one secondary subunit sequence beyond said portion; and
comparing an end portion of said reconstruction with subsequences corresponding to said primary subunit sequence to identify a subsequence which aligns best with said end portion of said reconstruction and which extends unambiguously in said alignment the reconstruction of said primary subunit sequence.

11. The method of claim 9, wherein said alternate reconstruction comprises deducing a best alignment between a subsequence and a sequence reconstruction by comparing the alignment of different subsequences with said reconstruction to produce a plurality of extended reconstructions together with individual alignment scores for each reconstruction, and optionally iteratively comparing downstream alignments of extended reconstructions using subsequences available for reconstruction, and determining a reconstruction with the highest scoring alignment to thereby deduce said best alignment.

12. The method of claim 6, further comprising generating subsequences using a sequence analysis technique.

13. The method of claim 6, wherein the sequence analysis technique is selected from a Sequence by Hybridization (SBH) technique or a shotgun sequencing technique.

14. The method of claim 6, wherein the at least one secondary subunit sequence is produced by mutagenesis of the primary subunit sequence.

15. The method of claim 6, wherein a secondary subunit sequence is produced by mutagenesis of another secondary subunit sequence.

16. The method of claim 14, wherein a parent subunit sequence is mutagenized to produce at least one variant subunit sequence in which at least 5% of subunits are different relative to the parent subunit sequence.

17. The method of claim 15, wherein a parent subunit sequence is mutagenized to produce at least one variant subunit sequence in which at least 5% of subunits are different relative to the parent subunit sequence.

18. The method of claim 6, wherein said subunit sequences are selected from amino acid sequences or nucleic acid sequences.

19. The method of claim 6, wherein said subunit sequences are selected from nucleic acid sequences.

20. The method of claim 19, wherein a parent nucleic acid sequence is mutagenized by incorporation of nucleotide analogues.

21. The method of claim 19, wherein a parent nucleic acid sequence is mutagenized by incorporation of nucleotide analogues, which are selected from dPTP (6-(2-deoxy-B-D-ribofuranosyl)-3,4-dihydro-8H-pyrimido-[4,5-C]oxazin-7-one triphosphate) or 8-oxo-dGTP (8-oxo-deoxyguanosine triphosphate).

22. The method of claim 19, wherein a parent nucleic acid sequence is mutagenized using low fidelity nucleic acid amplification reaction and an error prone DNA polymerase.

23. The method of claim 19, wherein a parent nucleic acid sequence is mutagenized using a repair deficient host.

24. A computer program product for wholly or partially deducing the sequence of a target subunit sequence, the computer program product including computer executable code which when implemented on a suitable processing system causes the processing system to perform the method of claim 1.

25. A computer program product for wholly or partially deducing the sequence of a target subunit sequence, the computer program product including computer executable code which when implemented on a suitable processing system causes the processing system to perform the method of claim 6.

26. A computer program product stored on a computer readable medium, the computer program product being adapted to cause a processing system to wholly or partially deduce the sequence of a target subunit sequence, the computer program product including computer executable code which when implemented on a suitable processing system causes the processing system to perform the method of claim 1.

27. A computer program product stored on a computer readable medium, the computer program product being adapted to cause a processing system to wholly or partially deduce the sequence of a target subunit sequence, the computer program product including computer executable code which when implemented on a suitable processing system causes the processing system to perform the method of claim 6.


SEQUENCE LISTING (188 sequences)

SEQ ID NO: 1 - DNA, 20 bp, Artificial
  Sequence string
cgcttcatgaatggtccgtt

SEQ ID NO: 2 - DNA, 18 bp, Artificial
  Sequence String
cgttgcttattcaagtgc

SEQ ID NO: 3 - DNA, 24 bp, Artificial
  Sequence string
caagaatattcggttgattactca

SEQ ID NO: 4 - DNA, 7 bp, Artificial
  Sequence string
gtagtga

SEQ ID NO: 5 - DNA, 25 bp, Artificial
  Sequence string
cagcatccgctgtgcctggaccaca

SEQ ID NO: 6 - DNA, 21 bp, Artificial
  Sequence string
gaaccacagtagtgaaatgac

SEQ ID NO: 7 - DNA, 20 bp, Artificial
  Sequence string
agccggggccacaggagtga

SEQ ID NO: 8 - DNA, 27 bp, Artificial
  Sequence string
gtatgcctggaaccctgtagtgatcag

SEQ ID NO: 9 - DNA, 50 bp, Artificial
  Sequence string
cctgagatcgcttcgtgaatggtccgttgcttattcaagtgctttaccac

SEQ ID NO: 10 - DNA, 5 bp, Artificial
  Sequence string
cctga

SEQ ID NO: 11 - DNA, 5 bp, Artificial
  Sequence string
cgctt

SEQ ID NO: 12 - DNA, 5 bp, Artificial
  Sequence string
gaatg

SEQ ID NO: 13 - DNA, 5 bp, Artificial
  Sequence string
cgttg

SEQ ID NO: 14 - DNA, 5 bp, Artificial
  Sequence string
attca

SEQ ID NO: 15 - DNA, 5 bp, Artificial
  Sequence string
cttta

SEQ ID NO: 16 - DNA, 5 bp, Artificial
  Sequence string
ctgag

SEQ ID NO: 17 - DNA, 5 bp, Artificial
  Sequence string
gcttc

SEQ ID NO: 18 - DNA, 5 bp, Artificial
  Sequence string
aatgg

SEQ ID NO: 19 - DNA, 5 bp, Artificial
  Sequence string
gttgc

SEQ ID NO: 20 - DNA, 5 bp, Artificial
  Sequence string
ttcaa

SEQ ID NO: 21 - DNA, 5 bp, Artificial
  Sequence string
tttac

SEQ ID NO: 22 - DNA, 5 bp, Artificial
  Sequence string
tgaga

SEQ ID NO: 23 - DNA, 5 bp, Artificial
  Sequence string
cttcg

SEQ ID NO: 24 - DNA, 5 bp, Artificial
  Sequence string
atggt

SEQ ID NO: 25 - DNA, 5 bp, Artificial
  Sequence string
ttgct

SEQ ID NO: 26 - DNA, 5 bp, Artificial
  Sequence string
tcaag

SEQ ID NO: 27 - DNA, 5 bp, Artificial
  Sequence string
ttacc

SEQ ID NO: 28 - DNA, 5 bp, Artificial
  Sequence string
gagat

SEQ ID NO: 29 - DNA, 5 bp, Artificial
  Sequence string
ttcgt

SEQ ID NO: 30 - DNA, 5 bp, Artificial
  Sequence string
tggtc

SEQ ID NO: 31 - DNA, 5 bp, Artificial
  Sequence string
tgctt

SEQ ID NO: 32 - DNA, 5 bp, Artificial
  Sequence string
caagt

SEQ ID NO: 33 - DNA, 5 bp, Artificial
  Sequence string
tacca

SEQ ID NO: 34 - DNA, 5 bp, Artificial
  Sequence string
agatc

SEQ ID NO: 35 - DNA, 5 bp, Artificial
  Sequence string
tcgtg

SEQ ID NO: 36 - DNA, 5 bp, Artificial
  Sequence string
ggtcc

SEQ ID NO: 37 - DNA, 5 bp, Artificial
  Sequence string
gctta

SEQ ID NO: 38 - DNA, 5 bp, Artificial
  Sequence string
aagtg

SEQ ID NO: 39 - DNA, 5 bp, Artificial
  Sequence string
accac

SEQ ID NO: 40 - DNA, 5 bp, Artificial
  Sequence string
gatcg

SEQ ID NO: 41 - DNA, 5 bp, Artificial
  Sequence string
cgtga

SEQ ID NO: 42 - DNA, 5 bp, Artificial
  Sequence string
gtccg

SEQ ID NO: 43 - DNA, 5 bp, Artificial
  Sequence string
cttat

SEQ ID NO: 44 - DNA, 5 bp, Artificial
  Sequence string
agtgc

SEQ ID NO: 45 - DNA, 5 bp, Artificial
  Sequence string
atcgc

SEQ ID NO: 46 - DNA, 5 bp, Artificial
  Sequence string
gtgaa

SEQ ID NO: 47 - DNA, 5 bp, Artificial
  Sequence string
tccgt

SEQ ID NO: 48 - DNA, 5 bp, Artificial
  Sequence string
ttatt

SEQ ID NO: 49 - DNA, 5 bp, Artificial
  Sequence string
gtgct

SEQ ID NO: 50 - DNA, 5 bp, Artificial
  Sequence string
tcgct

SEQ ID NO: 51 - DNA, 5 bp, Artificial
  Sequence string
tgaat

SEQ ID NO: 52 - DNA, 5 bp, Artificial
  Sequence string
ccgtt

SEQ ID NO: 53 - DNA, 5 bp, Artificial
  Sequence string
tattc

SEQ ID NO: 54 - DNA, 5 bp, Artificial
  Sequence string
gcttt

SEQ ID NO: 55 - DNA, 5 bp, Artificial
  Sequence string
cctga

SEQ ID NO: 56 - DNA, 13 bp, Artificial
  Sequence string
cctgagatcgctt

SEQ ID NO: 57 - DNA, 5 bp, Artificial
  Sequence string
gcttc

SEQ ID NO: 58 - DNA, 5 bp, Artificial
  Sequence string
gctta

SEQ ID NO: 59 - DNA, 5 bp, Artificial
  Sequence string
gcttt

SEQ ID NO: 60 - DNA, 4 bp, Artificial
  Sequence string
gctt

SEQ ID NO: 61 - DNA, 13 bp, Artificial
  Sequence string
cctgagatcgctt

SEQ ID NO: 62 - DNA, 16 bp, Artificial
  Sequence string
gcttattcaagtgctt

SEQ ID NO: 63 - DNA, 23 bp, Artificial
  Sequence string
gcttcgtgaatggtccgttgctt

SEQ ID NO: 64 - DNA, 10 bp, Artificial
  Sequence string
gctttaccac

SEQ ID NO: 65 - DNA, 50 bp, Artificial
  Sequence string
ccaaagatcgcttcaagaatattcggttgattactcaagagcctcaccac

SEQ ID NO: 66 - DNA, 7 bp, Artificial
  Sequence string
ccaaaga

SEQ ID NO: 67 - DNA, 23 bp, Artificial
  Sequence string
aagaatattcggttgattactca

SEQ ID NO: 68 - DNA, 10 bp, Artificial
  Sequence string
aagagcctca

SEQ ID NO: 69 - DNA, 15 bp, Artificial
  Sequence string
aagatcgcttcaaga

SEQ ID NO: 70 - DNA, 7 bp, Artificial
  Sequence string
ctcaaga

SEQ ID NO: 71 - DNA, 8 bp, Artificial
  Sequence string
ctcaccac

SEQ ID NO: 72 - DNA, 13 bp, Artificial
  Sequence string
cctgagatcgctt

SEQ ID NO: 73 - DNA, 7 bp, Artificial
  Sequence string
ccaaaga

SEQ ID NO: 74 - DNA, 13 bp, Artificial
  Sequence string
cctgagatcgctt

SEQ ID NO: 75 - DNA, 7 bp, Artificial
  Sequence string
ccaaaga

SEQ ID NO: 76 - DNA, 23 bp, Artificial
  Sequence string
aagaatattcggttgattactca

SEQ ID NO: 77 - DNA, 10 bp, Artificial
  Sequence string
aagagcctca

SEQ ID NO: 78 - DNA, 15 bp, Artificial
  Sequence string
aagatcgcttcaaga

SEQ ID NO: 79 - DNA, 13 bp, Artificial
  Sequence string
cctgagatcgctt

SEQ ID NO: 80 - DNA, 18 bp, Artificial
  Sequence string
ccaaagatcgcttcaaga

SEQ ID NO: 81 - DNA, 16 bp, Artificial
  Sequence string
gcttattcaagtgctt

SEQ ID NO: 82 - DNA, 23 bp, Artificial
  Sequence string
gcttcgtgaatggtccgttgctt

SEQ ID NO: 83 - DNA, 10 bp, Artificial
  Sequence string
gctttaccac

SEQ ID NO: 84 - DNA, 32 bp, Artificial
  Sequence string
cctgagatcgcttcgtgaatggtccgttgctt

SEQ ID NO: 85 - DNA, 18 bp, Artificial
  Sequence string
ccaaagatcgcttcaaga

SEQ ID NO: 86 - DNA, 23 bp, Artificial
  Sequence string
aagaatattcggttgattactca

SEQ ID NO: 87 - DNA, 16 bp, Artificial
  Sequence string
gcttattcaagtgctt

SEQ ID NO: 88 - DNA, 7 bp, Artificial
  Sequence string
ctcaaga

SEQ ID NO: 89 - DNA, 10 bp, Artificial
  Sequence string
aagagcctca

SEQ ID NO: 90 - DNA, 44 bp, Artificial
  Sequence string
cctgagatcgcttcgtgaatggtccgttgcttattcaagtgctt

SEQ ID NO: 91 - DNA, 46 bp, Artificial
  Sequence string
ccaaagatcgcttcaagaatattcggttgattactcaagagcctca

SEQ ID NO: 92 - DNA, 23 bp, Artificial
  Sequence string
gcttcgtgaatggtccgttgctt

SEQ ID NO: 93 - DNA, 10 bp, Artificial
  Sequence string
gctttaccac

SEQ ID NO: 94 - DNA, 7 bp, Artificial
  Sequence string
ctcaaga

SEQ ID NO: 95 - DNA, 8 bp, Artificial
  Sequence string
ctcaccac

SEQ ID NO: 96 - DNA, 10 bp, Artificial
  Sequence string
gctttaccac

SEQ ID NO: 97 - DNA, 8 bp, Artificial
  Sequence string
ctcaccac

SEQ ID NO: 98 - DNA, 50 bp, Artificial; identical to SEQ ID NO: 9
  Sequence string

SEQ ID NO: 99 - DNA, 50 bp, Artificial; identical to SEQ ID NO: 65
  Sequence string

SEQ ID NO: 100 - DNA, 4 bp, Artificial
  Sequence string
gctt

SEQ ID NO: 101 - DNA, 4 bp, Artificial
  Sequence string
gatt

SEQ ID NO: 102 - DNA, 4 bp, Artificial
  Sequence string
gcct

SEQ ID NO: 103 - DNA, 4 bp, Artificial
  Sequence string
aaga

SEQ ID NO: 104 - DNA, 4 bp, Artificial
  Sequence string
ctca

SEQ ID NO: 105 - DNA, 4 bp, Artificial
  Sequence string
gctt

SEQ ID NO: 106 - DNA, 50 bp, Artificial
  Sequence string
ttgagattcctatatatgttcgattcctatatatgaggattcctatataa

SEQ ID NO: 107 - DNA, 12 bp, Artificial
  Sequence string
gattcctatata

SEQ ID NO: 108 - DNA, 12 bp, Artificial
  Sequence string
ttgagattccta

SEQ ID NO: 109 - DNA, 14 bp, Artificial
  Sequence string
ttcctatatatgtt

SEQ ID NO: 110 - DNA, 16 bp, Artificial
  Sequence string
atatgttcgattccta

SEQ ID NO: 111 - DNA, 14 bp, Artificial
  Sequence string
ttcctatatatgag

SEQ ID NO: 112 - DNA, 13 bp, Artificial
  Sequence string
atatgaggattcc

SEQ ID NO: 113 - DNA, 12 bp, Artificial
  Sequence string
attcctatataa

SEQ ID NO: 114 - DNA, 12 bp, Artificial
  Sequence string
ttgagattccta

SEQ ID NO: 115 - DNA, 22 bp, Artificial
  Sequence string
ttcctatatatgttcgattcct

SEQ ID NO: 116 - DNA, 21 bp, Artificial
  Sequence string
attcctatatatgaggattcc

SEQ ID NO: 117 - DNA, 12 bp, Artificial
  Sequence string
attcctatataa

SEQ ID NO: 118 - DNA, 50 bp, Artificial; identical to SEQ ID NO: 106
  Sequence string

SEQ ID NO: 119 - DNA, 50 bp, Artificial
  Sequence string
ttgagattcctatatatgaggattcctatatatgttcgattcctatataa

SEQ ID NO: 120 - DNA, 50 bp, Artificial
  Sequence string
tttagattcctatatatattcgatttctgtatattaggaatcccatatta

SEQ ID NO: 121 - DNA, 13 bp, Artificial
  Sequence string
tttagattcctat

SEQ ID NO: 122 - DNA, 15 bp, Artificial
  Sequence string
ttcctatatatattc

SEQ ID NO: 123 - DNA, 18 bp, Artificial
  Sequence string
tatattcgatttctgtat

SEQ ID NO: 124 - DNA, 15 bp, Artificial
  Sequence string
ctgtatattaggaat

SEQ ID NO: 125 - DNA, 16 bp, Artificial
  Sequence string
taggaatcccatatta

SEQ ID NO: 126 - DNA, 12 bp, Artificial
  Sequence string
ttgagattccta

SEQ ID NO: 127 - DNA, 21 bp, Artificial
  Sequence string
attcctatatatgaggattcc

SEQ ID NO: 128 - DNA, 50 bp, Artificial; identical to SEQ ID NO: 120
  Sequence string

SEQ ID NO: 129 - DNA, 22 bp, Artificial
  Sequence string
ttcctatatatgttcgattcct

SEQ ID NO: 130 - DNA, 12 bp, Artificial
  Sequence string
attcctatataa

SEQ ID NO: 131 - DNA, 12 bp, Artificial
  Sequence string
gattcctatata

SEQ ID NO: 132 - DNA, 8 bp, Artificial
  Sequence string
cccgcgcc

SEQ ID NO: 133 - DNA, 8 bp, Artificial
  Sequence string
ggcgcggg

SEQ ID NO: 134 - DNA, 48 bp, Artificial
  Sequence string
cagaagaaggaacccgcgccctctgggctggcgcgggaggctcacacc

SEQ ID NO: 135 - DNA, 8 bp, Artificial
  Sequence string
cagaagaa

SEQ ID NO: 136 - DNA, 8 bp, Artificial
  Sequence string
ctcacacc

SEQ ID NO: 137 - DNA, 48 bp, Artificial
  Sequence string
cagatgaaggaaccctcgccctctgggctggcgcgggcgactcccacc

SEQ ID NO: 138 - DNA, 48 bp, Artificial
  Sequence string
aagaagaagaaaaccccgtccgctgggctgttgcgggagactcacagc

SEQ ID NO: 139 - DNA, 48 bp, Artificial
  Sequence string
caaaagaaggaacccgcaccctctgtgatctcgcgggaggctctcacc

SEQ ID NO: 140 - DNA, 48 bp, Artificial
  Sequence string
cagaacaaggaacccgcggcttctggactggcgcgagcggctcacatc

SEQ ID NO: 141 - DNA, 48 bp, Artificial
  Sequence string
cagaagaaggtacccgcgccttctgctctggtgcgggaggctcacacg

SEQ ID NO: 142 - DNA, 48 bp, Artificial; identical to SEQ ID NO: 134
  Sequence string

SEQ ID NO: 143 - DNA, 8 bp, Artificial
  Sequence string
cagaagaa

SEQ ID NO: 144 - DNA, 8 bp, Artificial
  Sequence string
ctcacacc

SEQ ID NO: 145 - DNA, 27 bp, Artificial
  Sequence string
cagatgaaggaaccctcgccctctggg

SEQ ID NO: 146 - DNA, 20 bp, Artificial
  Sequence string
aaaccccgtccgctgggctg

SEQ ID NO: 147 - DNA, 13 bp, Artificial
  Sequence string
aagaaggaacccg

SEQ ID NO: 148 - DNA, 22 bp, Artificial
  Sequence string
gatctcgcgggaggctctcacc

SEQ ID NO: 149 - DNA, 18 bp, Artificial
  Sequence string
cagaacaaggaacccgcg

SEQ ID NO: 150 - DNA, 20 bp, Artificial
  Sequence string
tggcgcgagcggctcacatc

SEQ ID NO: 151 - DNA, 28 bp, Artificial
  Sequence string
ccgcgccttctgctctggtgcgggaggc

SEQ ID NO: 152 - DNA, 9 bp, Artificial
  Sequence string
tgaaggaac

SEQ ID NO: 153 - DNA, 9 bp, Artificial
  Sequence string
agaagaaaa

SEQ ID NO: 154 - DNA, 9 bp, Artificial
  Sequence string
agaaggaac

SEQ ID NO: 155 - DNA, 9 bp, Artificial
  Sequence string
acaaggaac

SEQ ID NO: 156 - DNA, 9 bp, Artificial
  Sequence string
agaaggtac

SEQ ID NO: 157 - DNA, 251 bp, Artificial
  Sequence string
ggcgtaataatactatttgttgtgtcaattttcttggttcctgactaaaacattaaggtttctcagttaagctatatacgataaatattggcatctttctattgcaggatgatttctagtgctaagcattatagccaggagtaaaggaaataacgctttaacgataccaccattaatttaaaaaatggagtctgaaatggaaaaagaagaaaaaagcaatctcatctacgataaagatcctggatatgtgt

SEQ ID NO: 158 - DNA, 247 bp, Artificial
  Sequence string
ggcgtaataatactatttgttgtgtcaattgtcttggttcctgactaaaacattaaggtttctcagttaagctatagacgataaatattggcatctttctattgcaggatgatttctagtgctaagcattatagccaggagtaaaggaaataacgcgttaacgataccaccattaatttaaaaaatggagtctgaaatggaaaaagaagaaaaaagcaatctcatctacgataaagatcctggatat

SEQ ID NO: 159 - DNA, 247 bp, Artificial
  Sequence string
ggcgtaataatactatttgttgtgtcaatttgctgggttcctgactaacacattcaggtttctcagttaagctatatacgatacatatgggcatctttctattgcaggatgatttctagtgctacgcatgatagccaggagtaaaggaaataacgctttaacgctaccaccattaatttaaaaaatggagtctgaaagggaaaaagaagaaaaacgcnatctcatctacgataaagatcctggatat

SEQ ID NO: 160 - DNA, 247 bp, Artificial
  Sequence string
ggcgtaataatactatttgttgtgtcaattttcttggttcctgactaaaacattaaggtttctcagttaagctatatacgataaatcttggcatcttgctattgcaggatgatttctagtgctaagcagtatagccaggagtaaaggaaatcacgctttaacgataccaccattaatttaaaaaatggagtctgaactggaaaacgaagaaacaagcaatctcatctacgataaagatccgggatct

SEQ ID NO: 161 - DNA, 247 bp, Artificial
  Sequence string
ggcgtaataatactatgtgttgngtcaattttcttggttcctgactaaaacattaaggtttctcagttaagctatntacgataaatattggcatctttntattgcaggatgatttctagtgctaagcattatagccaggagtaaaggaaataacgctttaacgataccaccattaatttaaaaaatggagtctgnactggaaaaagaagaaaaaagcaatctcatctacgataaagatcctggatat

SEQ ID NO: 162 - DNA, 247 bp, Artificial
  Sequence string
ggcgtaataatactatttgttgtgtcaatctcctcggttcctgactaaaacattaaggtttctcagttaagctatacacgataaacactggcatccttctactgcaggatgatctccagtgctaagcattatagccaggagtaaaggaaataacgctttaacgataccaccattaatctaaaaaatggagcctggaatggaaaaagaagaaaaaagcaatctcatctacgataaagattctggatac

SEQ ID NO: 163 - DNA, 247 bp, Artificial
  Sequence string
ggcgtaataatactatccgttgtgtcaattttcttggttcctgactaaaacattaaggtttcttagttaagctatatacgataaatattggcacctttctattgcaggatgacttctagtgctaagcattatagtcaggagtaaaggaaataacgctttaacgataccaccattaattcaaaaaatggagtctgaaatggaaaaagaagaaaaaagcaacctcatctacgataaagatcctggatat

SEQ ID NO: 164 - DNA, 247 bp, Artificial
  Sequence string
ggcgtaataatactatttgttgtgtcaattttcttggttcctgactaaaacattaaggtttctcagttaagctatatacgataaatattggcatctttctattgcaggatgatttctagtgctaagcattatagccaggagtaaaggaaataacgctttaacgataccaccattaatttaaaaaatggagtctgaaatggaaaaagaagaaaaaagcaatctcatctacgataaagatcctggatat

SEQ ID NO: 165 - DNA, 247 bp, Artificial; identical to SEQ ID NO: 164
  Sequence string

SEQ ID NO: 166 - DNA, 245 bp, Artificial
  Sequence string
ggcgtaataacactatttgtcgtgccaattttcttggttcctggctaaagcattagggtctctcggttaggctgtatacggcgagtgttggcatcttcctatcgcgggatgatttctagtgctagacgctatagccaggggtaaaggaagtaacgcttcagcggtaccaccattagtttaaagggtggggcctgaagtggaaaaagggaaagaaagcaatctcatttcggnnaagatccnggttt

SEQ ID NO: 167 - DNA, 248 bp, Artificial
  Sequence string
gacgtagtagtactatctgtcgtgtcagtcttcctggttcccgaccaagacactaaggtctctcagctagactgtgcacgataaatattggcgccttcctactgcggaatgacttctaatgctaagcactatgaccaggagtggaggaggcaacactctaacgacaccaccattaattcaaagagcggagcctgggatggggagaggggaaaagagcaaccccatctacaataaaaganccttgattt

SEQ ID NO: 168 - DNA, 245 bp, Artificial
  Sequence string
gacgtagtagtactatttgctgtgccaaccttcttagtccctggctgaagcattgaggtttcccggtcaaaccatacgcgataagtattggcacctttctactacaggatggcttctagtgccagacattacagccaggggtgaaggggataacgctttagcgacaccaccgttaacctaaaagatggagtctgaaatggaaaaaggggagagaagcaatctcgcctacgacaaaaacttgatnc

SEQ ID NO: 169 - DNA, 247 bp, Artificial
  Sequence string
ggcgtgataacgctacctgctacaccaatcctcctggctcctggccaaggcactaaggtctctcagtcgggctatatacagtaggcattggcattcctttgtcgtggggtaatctctagtgctaaacattatagccaggggtgaaggaaataacgctctaacgataccaccgctagcccaaaaagtggagtccgggatggagaaagaggagaggagcaatcccgctgcantaaaggcccctggacat

SEQ ID NO: 170 - DNA, 245 bp, Artificial
  Sequence string
ggcgtaataataccacttgttgtgtcaatttccttggttcctgactaaaacactaaggtctttcagctaagccgtatacgataaacactggcatctttctgctacagggcgattcctagtgctaggcattatggccaggagtaaaggggatgacgcttcagcggcaccgccattggtttaaagaatggggcctgaaatggagaaagagggaagaggcaatctcatntgcgatagaagctggatat

SEQ ID NO: 171 - DNA, 245 bp, Artificial
  Sequence string
ggcgtaatgatactacctgtcgtgccaatcttcttggttcctgactagagcattaaggtttctcagttaagctatatacggtaaatattggcatcttcctattgcaggatgatttctagtgccgagcattatagccaggagtaaaggaaatggcgccttagcggtgccaccattagtttaaagaatggagtctgaaatggaaaaagaaggaaaaagcagccttatctacgataaggactgagtat

SEQ ID NO: 172 - DNA, 245 bp, Artificial
  Sequence string
ggcgtagtaataccatctactgtgtcaatcccctcgactcccgactgaaacattaaggtttctcagctaagctacgcacgatgagtaccggcatccctctatcgcaggacgatccctagtgctaggcatcacagccgggagtaagggagataacactttaacggtaccaccactaactcggagaatggagtttgaagtggaaaagggggaaaaaaacaatctcgtctgcggtagaggccgggcgt

SEQ ID NO: 173 - DNA, 245 bp, Artificial
  Sequence string
ggcgtaacagtactacttgctgcgtcaactttcttggtccctggctgaagcgttaaggcctctcggttgagctatgcacggtaaatactgacgcttccccgtcgcagggcgatctntggcgccaagcatnatagccaggggtaaaggaagtaacgctttggcggcaccaccactaacttagagaatggagtccgggatgggggaaggggaaagagacgaccccacctacggtggggaccgggtat

SEQ ID NO: 174 - DNA, 245 bp, Artificial
  Sequence string
ggcgtaataatactatctgttgtgtcaattttcttggttcctgactaaagcattaaggtttctcagttaagctgtatacgataaatattggcacctttctattgcaggatgatttctggtgctaagcattacagccaggagtaaaggaaataacgctttaacggtaccaccattaatttaaaaaatggaatntgaaatggaaaaagaagagaaaagcaatctnatctacggtnaagactgggtat

SEQ ID NO: 175 - DNA, 245 bp, Artificial
  Sequence string
ggcgtaataatactacctgttgcgccaatctccctggtccctgactaaaacgttaaggtctcccagttaagccacatacgacagatattggcatccccctactgcggaatgatttctggtgctaagcgttatagccaggagtaaagggaatgacgccttaacggtaccgccgttaattcaaagagtggagtctggagtgagaaaaggagaaggaagcagtcccatctgcaataagggccgggtat

SEQ ID NO: 176 - DNA, 245 bp, Artificial
  Sequence string
ggcgtaataatactacttactgtgttaattctctcggtccccgactaaaacattaaggtttcccagttaagctatatatggtaaatgccggcacctttctatcgcaggatgatctctagcgccaggcgctatagtcaggagtagaggagatgacgctttaacgataccgccatcaacttgagaagtggagtctgaaacggaggaggaaggaaaaaataatctcattcacgatagaaactggatat

SEQ ID NO: 177 - DNA, 185 bp, Artificial
  Sequence string
ggcgtaataatactgtttgttgtgtcaattttcttggttcctgaccaaaacattaaagttgctaagcatcatagtcaggagtaaaggggataacgctttggcgataccaccattaatctaaaaagtggagtctgaaacggaagaagaggaagagagtaatctcatctacggcaaaggctgggtat

SEQ ID NO: 178 - DNA, 245 bp, Artificial
  Sequence string
aacgtaataatactatttgccgtgtcaattttctcggtccccgactgaaatgttagggtttcccagttaagctatatacgataaatattggcatctttccattgcgggatgattcctagtgctaagcattatagccaggagtaaaggaaataacgctttagcagtaccaccattaatttaaaagatggagtctgaagtggaaaaggaggaaaaaagcaatctcatctacgataaagactggatac

SEQ ID NO: 179 - DNA, 246 bp, Artificial
  Sequence string
ggcacaataatattatttgtcgngtcagtctctctggctcctgaccaaaacgtcaaggtcccccagtcaagccacatacgacagacattggcatcttcctactacaggatgatttctagtgctaagtgttgtagccaggagcaaaggagataacgccctaatggcgccatcattaattcagaaagtggagcctaaagtggagaaagagaagaagagtaccccgtctacagtnaagaccccgggtat

SEQ ID NO: 180 - DNA, 245 bp, Artificial
  Sequence string
ggcgtaacagcaccacttgttgtgccgattcccttggtccccgactaagacactagggtcccccagtcagaccatatacgataaataccggcacctccttaccgcgggacagttcctaacgctaagcactatagccgggggtaaaggggataacgcttcgacgataccaccgctaacttaagggatggggcctgagatggagagagaagaagagagcgatctcatctacgataagggctggatat

SEQ ID NO: 181 - DNA, 245 bp, Artificial
  Sequence string
ggcgtaatagtactatttgttgtgtcaattctcttggctcctgactgaaacactaaggtctctcagctaggctatgtgcgacggatattggcatccttctgctacaggatgacttctagcgctgggcgccatagccaggagtaaaggggataatgctctaacggcaccaccactaatttaagaaatggagtctaaagtgggaaaagaagaaaagagcaatctcacccacgacgaggactggatat

SEQ ID NO: 182 - DNA, 245 bp, Artificial
  Sequence string
aacgtaatagtaccattcgttgtgtcaactttcttggcccctgactagagcatcgaggtctcttggttaagctgcatgcgataaatattggcgcctctctactgcagggtggtccctggngctgggcgttatagccgggagtaagggagacaacgcttnggcggcaccaccactagtttaaggaatggagtctgaaacggaaggagaagagaaaggcaatcccatctacaataaagactgggtat

SEQ ID NO: 183 - DNA, 245 bp, Artificial
  Sequence string
ggcgtagcggtactgttcgctgtgtcgatcttcttggcccctgactagagcattaaggtctcttgattaagctatgcacggtgaatactggcatcctcctatcgcaggacggcctctggtgctgagcgctatggccaggagtgaaggggatgacgccttaacagtaccaccgttagttcagaaagcggagtctgagatggaggaggaaggaagaagcaatcttatctacggtaaagactggatat

SEQ ID NO: 184 - DNA, 245 bp, Artificial
  Sequence string
ggcgtagtaatactacttgttgtgtcaactttcttggtccctgactaaaacattaaggtttcccagctaagtcatatacgataaatactggcgttcttctactgcgggacggctcctggtgctaagcgttatagccaggagtggaggaaataacgctttagcgataccaccattaatttaaaaaatggagtctgagatgggagaggaagaagaaaacagtctcacctacgacaaggactggatat

SEQ ID NO: 185 - DNA, 195 bp, Artificial
  Sequence string
cattaaggtccctcggttgagctatgtacggtgagtattggcgccttcctattgcagaataatttctagcgccaagcattgtagccgggggtaaaggaaacagcgcttcaacgataccgccactaactcaaagaatggagtctggagtggagaaaaggggagagagcagtcccatccacggtaaagactggatat

SEQ ID NO: 186 - DNA, 245 bp, Artificial
  Sequence string
ggcgtaataatactatttgttgtgtcaattttcttggttcctgactaaaacattaaggtttctcagttaagctatatacgataaatattggcatctttctattgcaggatgatttctagtgctaagcattatagccaggagtaaaggaaataacgctttaacgataccaccattaatttaaaaaatggagtctgaaatggaaaaagaagaaaaaagcaatctcatctacgataaagactggatat

SEQ ID NO: 187 - DNA, 245 bp, Artificial; identical to SEQ ID NO: 186
  Sequence string

SEQ ID NO: 188 - DNA, 94 bp, Artificial
  Sequence string
gctccagtggcgcaatcggttagcgcgcggtacttatacaacagtatatgtgcgggtgatgccgaggttgtgagttcgagcctcacctggagca